(19) United States
(12) Patent Application Publication
(10) Pub. No.: US 2014/0249097 A1
(43) Pub. Date: Sep. 4, 2014

(54) Inhibition of HIF-1 activation for Anti-Tumor and Anti-Inflammatory responses

(71) Applicants: Mark W. Dewhirst, Durham, NC (US); Yiting Cao, Durham, NC (US)

(72) Inventors: Mark W. Dewhirst, Durham, NC (US); Yiting Cao, Durham, NC (US)

(21) Appl. No.: 13/200,819

(22) Filed: Sep. 30, 2011

Related U.S. Application Data
(60) Provisional application No. 61/404,267, filed on Sep. 30, 2010.

(51) Int. Cl. A61K 31/353; A61K 31/155; A61K 31/704

(52) U.S. Cl. CPC A61K 31/353; A61K 31/704; A61K 31/155
USPC 514/34

(57) ABSTRACT

The presently disclosed subject matter generally relates to methods and compositions for inhibiting the expression and/or activation of hypoxia-inducible factor 1 (HIF-1) genes in a cancer cell, tissue or tumor. More particularly, the methods disclosed herein relate to inhibition of HIF-1 activation in a tumor, increasing sensitivity of a tumor cell to radiation and/or chemotherapy, delaying tumor growth, inhibiting tumor blood vessel growth, inhibiting inflammatory responses in a cell through the use of compositions that prevent the nitrosylation of HIF-1, and methods for screening for new inhibitors of HIF-1 activation. Additionally, the compositions disclosed herein relate to compositions that can be employed in, and are identified by, the disclosed methods.

### CROSS-REFERENCE TO RELATED APPLICATION

This application claims benefit of U.S. Provisional Patent Application Ser. No. 61/404,267, entitled “Inhibition of HIF-1 Activation for Anti-tumor and Anti-inflammatory Responses”, filed Sep. 30, 2010, which is herein incorporated by reference in its entirety.

### GOVERNMENT INTEREST

The presently disclosed subject matter was made with U.S. Government support under Grant No. EB001882 from the U.S. National Institute of Bioimaging and Bioengineering, Grant No. CA81512 from the U.S. National Cancer Institute, Grant No. DAMD17-02-0052 from the U.S. Department of Defense, and Grant No. 5P50CA068438-10 from the U.S. National Institute of Health. Thus, the United States Government has certain rights in the presently disclosed subject matter.

### TECHNICAL FIELD

The presently disclosed subject matter generally relates to methods and compositions for treating tumors and cancers. More particularly, the presently disclosed subject matter provides methods and compositions for inhibiting the expression and/or activation of hypoxia-inducible factor 1 (HIF-1) gene products in a tumor or cancer cell undergoing cancer treatment.

### BACKGROUND

In a typical clinical setting, radiation therapy and/or chemotherapy treatments are administered to the majority (>90%) of cancer patients. Therefore, along with surgery, radiation therapy and chemotherapy represent two of the three main modalities employed for cancer treatment. However, the therapeutic outcomes are still far from ideal for many types of tumors. A significant problem associated with radiotherapy is the recurrence of tumors and/or the development of metastases at distant locations. For chemotherapy, a problem is the development of resistance. In both cases, new methods and compositions that can sensitize tumors to current treatments are highly desirable. Ideally, these methods and compositions should decrease local recurrences in patients treated with radiotherapy and/or should increase the efficacy of chemotherapeutic agents systemically. In addition, they should not have severe side effects.

What are needed, then, are new strategies and compositions for treating tumors and/or cancers via inhibition of HIF-1 activity and/or the upregulation of HIF-1 activity that results from radiotherapy and/or chemotherapy. The presently disclosed subject matter addresses this and other needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments the presently disclosed subject matter provides a method for implementing a treatment strategy for a cancer in a subject in need of treatment, the method comprising: administering to the subject a chemotherapeutic agent; determining a level of HIF-1 activity in a cancer tissue in the subject; and administering an inhibitor of HIF-1 activity to the subject if an elevation of HIF-1 activity is observed after administering the chemotherapeutic agent. In some embodiments the cancer tissue expresses inducible nitric oxide synthase (iNOS). In some embodiments the cancer tissue in which HIF-1 activity is evaluated is normoxic cancer tissue. In some embodiments the level of HIF-1 activity in the cancer tissue is determined by detecting a plasma level of plasminogen activator inhibitor-1 (PAI-1). In some embodiments the level of HIF-1 activity in the cancer tissue is determined by establishing a ratio between PAI-1 and osteopontin (OPN). In some embodiments the inhibitor of HIF-1 activity is selected from the group consisting of a nitric oxide synthase inhibitor, a nitric oxide scavenger, a STAT-1 inhibitor, and combinations thereof. In some embodiments the nitric oxide synthase inhibitor comprises an iNOS inhibitor. In some embodiments the chemotherapeutic agent acts by oxidative stress. In some embodiments the chemotherapeutic agent comprises doxorubicin. In some embodiments determining a level of HIF-1 activity in a cancer tissue in the subject is performed within 1 to 10 days after administering to the subject a chemotherapeutic agent. In some embodiments administering an inhibitor of HIF-1 activity to the subject is performed within 1 to 10 days after observing an elevation of HIF-1 activity. In some embodiments the subject is a mammal. In some embodiments the mammal is a human.

In some embodiments the presently disclosed subject matter provides a method of treating a cancer in a subject, the method comprising: administering a chemotherapeutic agent to the subject; determining whether administering the chemotherapeutic agent to the subject elevates HIF-1 activity in cancer tissue of the subject; and administering a HIF-1 inhibitor to the subject depending on whether elevated HIF-1 activity is observed in the cancer tissue. In some embodiments the cancer tissue expresses inducible nitric oxide synthase (iNOS). In some embodiments the cancer tissue in which HIF-1 activity is evaluated is normoxic cancer tissue. In some embodiments the level of HIF-1 activity in the cancer tissue is determined by detecting a plasma level of plasminogen activator inhibitor-1 (PAI-1). In some embodiments the level of HIF-1 activity in the cancer tissue is determined by establishing a ratio between PAI-1 and osteopontin (OPN). In some embodiments the HIF-1 inhibitor is selected from the group consisting of a nitric oxide synthase inhibitor, a nitric oxide scavenger, a STAT-1 inhibitor, and combinations thereof. In some embodiments the nitric oxide synthase inhibitor comprises an iNOS inhibitor. In some embodiments the chemotherapeutic agent acts by oxidative stress. In some embodiments the chemotherapeutic agent comprises doxorubicin. In some embodiments determining a level of HIF-1 activity in a cancer tissue in the subject is performed within 1 to 10 days after administering to the subject a chemotherapeutic agent. In some embodiments administering an inhibitor of HIF-1 activity to the subject is performed within 1 to 10 days after observing an elevation of HIF-1 activity. In some embodiments the subject is a mammal. In some embodiments the mammal is a human.

This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description and non-limiting Examples.

### BRIEF DESCRIPTION OF THE DRAWINGS

The patent or application file contains at least one drawing executed in color. Copies of this patent or patent application publication with color drawing(s) will be provided by the United States Patent and Trademark Office upon request and payment of the necessary fee.

FIGS. 1A-1D depict the results of experiments to establish ODD-luc as a non-invasive reporter for HIF-1α expression.

FIG. 1A depicts the domain structure of murine HIF-1α and reporter proteins. The oxygen dependent degradation (ODD) domain is located between amino acids 401 to 613 (top construct) of murine HIF-1α (SEQ ID NO: 3). The ODD-luc reporter coding sequence (middle construct) was obtained by inserting the ODD domain of murine HIF-1α between an upstream cytomegalovirus (CMV) promoter and the downstream gene firefly luciferase coding sequence (luc). The ODD and luc sequences were engineered to be in frame. A Kozak sequence and start codon (ATG) were inserted 5′ of the ODD coding sequence. A luciferase coding sequence driven by the CMV promoter (lower construct) was used as a control.

FIG. 1B depicts the expression of ODD-luc in 4T1 cells determined from luc-dependent conversion of luciferin to luminescent oxoluciferin, and measurement of associated light emission. The top panel depicts representative photographs obtained through the Xenogen IVIS™ system of ODD-luc expression/activity in 4T1 cells cultured after activation of ODD-luc by CoCl₂ (240 μM for 12 hrs), exposure to a proteasome inhibitor (10 μM MG132 for 12 hrs), hypoxia (0.5% O₂ for 24 hrs), or anti-VHL siRNA transfection (VHL-KD). A color ladder is provided to the right of the panel. The average luc activities were calculated from triplicate experiments in each case. Significant differences were observed between control and treated cells (p<0.05 in all cases, Student's t test). The bottom panel presents a graphical depiction of the activity levels presented in the photographic depictions in the top panel.

FIG. 1C depicts Western blot analysis showing down regulation of VHL protein expression after introducing an siRNA-expressing vector encoding an anti-VHL minigene into 4T1 cells as a stable, integrated construct. The sequence of the siRNA was AACATCACATTGCCAGTGTAT (SEQ ID NO: 17). β-actin levels were used as loading controls.

FIG. 1D depicts Western blot analysis of wild type 4T1 cells treated as in FIG. 1B. Lysates of the cells were analyzed for endogenous HIF-1α protein expression using a rabbit anti-mouse HIF-1α polyclonal antiserum. VHL-KD: cells stably transduced with an siRNA gene against VHL. β-actin was used as the loading control.

FIGS. 2A-2C depict in vivo activation of HIF-1α by ionizing radiation in tumors.

FIG. 2A depicts luciferase activity in 4T1 tumors stably transduced with ODD-luc or CMV-luc reporter genes established in nude mice. Size-matched tumors were locally irradiated (6 Gy) at day 0. Luciferase activity in tumors was determined daily though non-invasive imaging. Fourteen animals were used in each group and the error bars indicate the standard error of the mean. The difference between the irradiated group and control was significant (p<0.05 from day 3 to day 10 by two-way ANOVA).

FIG. 2B is a bar graph presenting the results of radiation-induced activation of endogenous HIF-1 binding activity to a hypoxia responsive element (HRE) measured by ELISA in tumors irradiated 5 days earlier. In each group, the average results from 4 tumor samples are shown (p<0.05, Student's t test). Error bars represent standard deviation.

FIG. 2C is a bar graph presenting the results of a radiation-induced increase in intratumoral VEGF levels as measured by ELISA. In each group, the average results from 4 tumors are shown (p<0.05, Student's t test). Error bars represent standard deviation.

FIGS. 3A-3D depict the results of experiments showing that nitric oxide is a key regulator of radiation-induced HIF-1α activation.

FIG. 3A presents the results of assays for luciferase activity in 4T1-ODD-luc or 4T1-luc transduced tumors established in the hind legs of nude mice and irradiated (at day 0) with or without the administration of L-NAME (at day −1). Luciferase levels were then monitored post irradiation. Tumors with the CMV-luc reporter were used as controls. Significant inhibition of ODD-luc expression were observed by the use of L-NAME. Eight animals were used in each group and the error bars indicate standard error of the mean. p<0.05 from day 4 (two-way ANOVA). The top panel is a graph of the activities of the listed conditions from day 0 to day 10, and the bottom panel depicts representative photographs at various stages and under the listed conditions.

FIG. 3B is a bar graph depicting S-nitrosoglutathione-(GSNO) induced activation of ODD-luc cells in vitro. 4T1-ODD-luc cells were exposed to the NO donor GSNO at indicated dosage and monitored for ODD-luc expression. The data were normalized against cells that were not treated with GSNO. The error bars represent standard deviations. Each data point represents the average of triplicate experiments. Dose-dependent induction was observed. p<0.001 (Student's t test)

FIG. 3C depicts Western blot analysis of endogenous HIF-1α protein levels after GSNO treatment (1 mM for 8 hours) in 4T1 cells. β-actin levels were used as loading control.

FIG. 3D is a bar graph depicting suppression of NO-mediated HIF-1α activation by a nitric oxide scavenger. 4T1-ODD-luc cells were exposed to GSNO (1 mM) in the presence or absence of a chemical NO scavenger—carboxy-PTIO (0.5 mM). The cells were monitored for luciferase expression 24 hours later. The error bars represent standard deviation and each data point represents the average of triplicate experiments. p<0.05 (Student's t test).

FIGS. 4A and 4B are graphical representations demonstrating the role of the inducible form of nitric oxide synthase (iNOS) in radiation-induced HIF-1α activation.

FIG. 4A depicts the effect of an iNOS specific inhibitor. Subcutaneous tumors were established in the hind legs of nude mice through the use of 4T1-ODD-luc cells and irradiated with or without the administration of 1400W, an iNOS-specific inhibitor. ODD-luc level were then monitored daily post irradiation. Significant inhibition of radiation-induced HIF-1 activation was observed in the group treated with 1400W (p<0.001 from day 4, two-way ANOVA).

FIG. 4B depicts the effect of a homozygous genetic disruption (i.e., knockout) of the iNOS gene on HIF-1α activation in a host animal. Tumors were established from B16F10-ODD-luc cells in syngeneic wild type or iNOS^{-/-} C57BL/6 mice. In some groups, mice received L-NAME one day before tumor irradiation (6 Gy) at day 0. Luciferase activities were determined every other day. Eight animals were used in each group and the error bars represent the standard errors of the mean. In wild type C57BL/6 mice (solid lines), the difference between L-NAME treated and non-treated groups was statistically significant (p<0.01 on days 1, 3, and 5, two-way ANOVA test). In iNOS^{-/-} mice (broken lines), the difference between L-NAME treated and non-treated groups was not significant (p>0.05 at all time points, two-way ANOVA).

FIGS. 5A and 5B depict the role of macrophages in radiation-induced HIF-1α activation.

FIG. 5A depicts luciferase expression in tumors established from 4T1-ODD-luc cells in nude mice. In some mice, macrophages were depleted by injection of carrageenan. Selected groups of mice also received L-NAME one day before irradiation (6 Gy). Luciferase expression was determined every other day. Eight mice were included in each group. The error bars represent the standard errors of the mean.

FIG. 5B depicts immunohistochemistry analysis of HIF-1α, iNOS, and macrophages in tumors. Mice with irradiated 4T1 tumors were sacrificed and their tumors excised 5 days after localized 6 Gy or sham irradiation of tumors. Shown in the left panel are representative results from co-staining of CD68 (a marker for macrophages (MO and iNOS. Co-staining of HIF-1α and iNOS is shown on the right panel. In each case, merged pictures are provided. Orange color in both panels represents co-localization.

FIGS. 6A-6F depict normoxic prevention of HIF-1α degradation though S-nitrosylation of cysteine 533.

FIG. 6A presents amino acid subsequence conservation across different species in the region of Cys 533 of murine HIF-1α (GENBANK® Accession No. NP_{—}034561 (SEQ ID NO: 3). The subsequences presented include PNSPSEYCFYVDSDM (Homo sapiens; SEQ ID NO: 18); PNSPSEYCFDVDSDM (Mus musculus; SEQ ID NO: 19); PNSPSEYCFDVDSDM (Rattus norvegicus; SEQ ID NO: 19); PNSPSEYCFDVDSDM (Spalax judaei; SEQ ID NO: 19); PNSPSEYCFDVDSDM (Bos grunniens; SEQ ID NO: 19); PNSPMEYCFQVDSDI (Carassius carassius; SEQ ID NO: 20); and EPNTPEYCFDVDSEM (Xenopus laevis; SEQ ID NO: 21). See also FIGS. 8a-8H.

FIG. 6B is a bar graph depicting the effects of various stimuli (0.5% hypoxia, proteasome inhibitor MG132, and CoCl₂) on the activation of wild type ODD-luc and C533S-ODD-luc in 4T1 cells. The experiments were carried out in the similar manner as those described in FIG. 1B. The data shown are the results of triplicate experiments. The error bars represent standard deviations. In all treatment groups, p>0.05 between wild type and mutant ODD-luc expression levels (Student's t test).

FIG. 6C is a bar graph depicting luciferase activity in wild type ODD-luc or C533S ODD-luc transduced 4T1 cells treated with GSNO (1 mM). Significant attenuation of luc expression was observed in C533S-ODD-luc transduced cells (p<0.01, Student's t test). Each data point is the results of triplicate experiments and the error bars represent standard errors. The unit for light output shown is p/sec/CM²/Sr.

FIG. 6D is a graph depicting luciferase activity in irradiated (6 Gy) tumors established from 4T1 cells transduced with wild type or C533S-ODD-luc. Luciferase expression was monitored every other day. Significant attenuation of luciferase expression was observed in C533S-ODD-luc-transduced 4T1 tumors (p<0.01 from day 5, two-way ANOVA). Each group has five animals and the error bars represent standard errors of the mean.

FIG. 6E depicts the results of Western blot analysis of S-nitrosylation of C533 in the ODD domain. 4T1 cells transduced with wild type ODD or C533S-ODD (both with a myc-tag at the 3′ end for Western blot detection) were exposed to GSNO and then lysed. S-nitrosylation of ODD was determined through the biotin switch assay (Jaffrey & Snyder, 2001). A clear nitrosylation signal was observed for wild type ODD after GSNO treatment, but was not observed in C533S ODD with or without GSNO treatment.

FIG. 6F depicts the results of Western blot analysis demonstrating the absence of binding between nitrosylated ODD and VHL. 4T1 tumor ells were transduced with CMV-ODD-mycTag, CMV-0533S-ODD-mycTag, or CMV-HA-VHL. Where indicated, ODD-transfected cells were exposed to 1 mM GSNO for 8 hours. The lysate of ODD-transfected cells was admixed with lysate of cells expressing HA-VHL. Mixed lysates were immunoprecipitated with anti-HA antibody to pull down the VHL protein and any ODD bound thereto. The immunoprecipitate was then immunoblotted with antibody against mycTag to detect ODD bound to VHL. Total tagged ODD (Input ODD) and VHL (Input VHL) were detected by Western blot analysis with antibodies against the mycTag and the HA-tag, respectively.

FIGS. 7A-7C depict the enhanced anti-tumor efficacy of radiotherapy in combination with L-NAME. B16F10 and 4T1 tumors were established in syngeneic C57BL/6 and 4T1 mice, respectively and irradiated with 3 fractions of X-rays at 6 Gy/fraction (irradiation every other day). In some of the groups, L-NAME was administered in the drinking water one day before irradiation. Tumor sizes were monitored every other day. At least 6 animals were used in each treatment groups. Tumor sizes were then plotted against time for each tumor type. The error bars represent the standard errors of the mean.

FIG. 7A is a graph depicting 4T1 tumor growth delay.

FIG. 7B is a graph depicting B16F10 melanoma growth delay.

FIG. 7C is a bar graph depicting CD31⁺ cells (indicative of vasculature) in tumors excised from different groups on day 10. The tumors were excised, sectioned, and probed for the presence of vasculature by use of an antibody against CD31, which stained for endothelial cells. The average vascular length density of tumors was determined from five randomly chosen fields for each treatment type. The error bars represent the standard errors. The differences between the combined treatment group and the individual groups were significant (p<0.05, one way ANOVA) in both tumor models.

FIGS. 8A through 8H present a maximized amino acid sequence alignment of HIF-1α polypeptide sequences from the following organisms: Spalax judaei (GENBANK® Accession No. CAG29396; SEQ ID NO: 1); Eospalax baileyi (GENBANK® Accession No. ABB17537; SEQ ID NO: 2); Mus musculus (GENBANK® Accession No. NP_{—}034561; SEQ ID NO: 3); Rattus norvegicus (GENBANK® Accession No. NP_{—}077335; SEQ ID NO: 4); Microtus oeconomus (GENBANK® Accession No. AAY27087; SEQ ID NO: 5); Homo sapiens (GENBANK® Accession No. NP_{—}001521; SEQ ID NO: 6); Pongo pygmaeus (GENBANK® Accession No. CAH93355; SEQ ID NO: 7); Macaca fascicularis (GENBANK® Accession No. BAE01417; SEQ ID NO: 8); Spermophilus tridecemlineatus (GENBANK® Accession No. AAU14021; SEQ ID NO: 9); Bos taurus (GENBANK® Accession No. NP_{—}776764; SEQ ID NO: 10); Pantholops hodgsonii (GENBANK® Accession No. AAX89137; SEQ ID NO: 11); Canis familiaris (GENBANK® Accession No. XP_{—}852278; SEQ ID NO: 12); Oryctolagus cuniculus (GENBANK® Accession No. AAP43517; SEQ ID NO: 13); Gallus gallus (GENBANK® Accession No. NP_{—}989628; SEQ ID NO: 14); Danio rerio (GENBANK® Accession No. NP_{—}956527; SEQ ID NO: 15); and Xenopus laevis (GENBANK® Accession No. CAB96628; SEQ ID NO: 16).

FIGS. 9A and 9B depict ODD-LUC expression in 4T1 tumor cells that have been treated with cyclophosphamide.

FIG. 9A depicts a time course of ODD-luc change in 4T1 tumors treated with cyclophosphamide.

FIG. 9B depicts images of ODD-luc expression with (bottom 2 panels) or without (top 2 panels) cyclophosphamide exposure.

FIGS. 10A-10E depict the upregulation of HIF-1α reporter activity in 4T1 ODD-luc tumor cells administered doxorubicin in vitro and in vivo.

FIG. 10A depicts bioluminescent images demonstrating that doxorubicin treatments (0.1, 1, and 10 μg/ml) activated HIF-1α reporter activity in normoxic 4T1 ODD-luc cells in vitro.

FIG. 10B depicts the quantitative analysis of the bioluminescent images of FIG. 10A and reveals that the doxorubicin treatments significantly increased the HIF-1α reporter activity compared to the control treatment. 1 μg/ml doxorubicin treatment induced the highest HIF-1α reporter gene activity 24 hours and 48 hours post-treatment. Bars, SE. *, P<0.05, one-way ANOVA, n=3.

FIG. 10C depicts Western blot analysis demonstrating normoxic HIF-1α accumulation in 4T1ODD-luc cells and MCF-7 cells 48 hours post-treatment.

FIG. 10D depicts bioluminescent images revealing that doxorubicin treatment caused higher HIF-1 reporter activity than the saline control treatment in orthotopic 4T1ODD-luc tumors 4 days post-treatment. Because the bioluminescence intensity of doxorubicin-induced HIF-1α reporter was so strong, the unit for the images in the doxorubicin group was greater than that in the saline group to avoid signal saturation.

FIG. 10E depicts the quantitative analysis of the bioluminescent images of FIG. 10D, which demonstrated that doxorubicin treatment significantly increased HIF-1α reporter activity compared to the saline control treatment on post-treatment days 3, 4, 5, and 13 in orthotopic 4T1ODD-luc tumors. Bars, SE. *, P<0.01, Student's t-test, n=7.

FIGS. 11A-11C depict multiple time-point immunohistochemical analysis of tumor HIF-1α expression and hypoxia in orthotopic 4T1ODD-luc tumors treated with doxorubicin versus saline.

FIG. 11A immunohistochemical stains of tumor HIF-1α expression and hypoxia in orthotopic 4T1ODD-luc tumors treated with doxorubicin versus saline. Maximal tolerated dose (MTD) doxorubicin chemotherapy increased tumor HIF-1α expression but not tumor hypoxia as detected by pimonidazole staining in comparison to the saline control treatment on post-treatment day 5. Bars, 1 mm.

FIG. 11B is a graphical representation demonstrating that MTD doxorubicin treatment significantly increased tumor HIF-1α fraction in comparison to the saline control treatment on post-treatment days 2, 4, 7, and 10. Bars, SE. *, P<0.05, **, P<0.01, Student's t-test, n=5.

FIG. 11C is a graphical representation demonstrating that no significant difference in pimonidazole fraction between doxorubicin-treated tumors and saline-treated tumors. Bars, SE. P>0.05, Student's t-test, n=5.

FIGS. 12A-12C depict the effects of doxorubicin on tumor cell VEGF secretion and in vivo tumor angiogenesis.

FIG. 12A is a bar graph depicting the results of a VEGF ELISA. Doxorubicin treatments (0.1, 1, and 10 μg/ml) stimulated 4T1ODD-luc cells to secret more VEGF in vitro in comparison to the control treatment. Bars, SE. *, P<0.001, one-way ANOVA, n=3.

FIG. 12B depicts fluorescent staining images revealing that MTD doxorubicin treatment induced more vasculature in orthotopic 4T1ODD-luc tumors than the saline control treatment on post-treatment day 4. Bar, 100 μm.

FIG. 12C is a graphical representation comparing the relative tumor vascular fraction between the doxorubicin-treated 4T1ODD-luc tumors and the saline-treated control tumors. Bars, SE. *, P<0.05, **, P<0.01, Student's t-test, n=5.

FIGS. 13A-13D illustrate that nitric oxide (NO) and nitric oxide synthase play important roles in doxorubicin-induced normoxic HIF-stabilization.

FIG. 13A is a bar graph depicting the flow cytometry analysis of fluorescent NO indicator DAF-FM. The results indicate that doxorubicin treatments increased intracellular NO levels. Results were normalized to the mean value of the control group. Bar, SE. *, P<0.05; **, P<0.001, One-way ANOVA, n=3. Dox, doxorubicin.

FIG. 13B depicts images of crystal violet staining and bioluminescent imaging demonstrating that, without doxorubicin treatment, L-NAME treatments did not inhibit the basal HIF-1α reporter activity in 4T1ODD-luc cells. In contrast, L-NAME treatments suppressed the upregulation of HIF-1 reporter induced by 1 μg/ml doxorubicin.

FIG. 13C is a bar graph depicting that without doxorubicin, the increased concentrations of L-NAME did not change the basal level of HIF-1 reporter activity in normoxic 4T1 ODD-luc cells over a 72-hour period. Bars, SE. P>0.05, one-way ANOVA, n=3.

FIG. 13D is a bar graph depicting that L-NAME led to a dose-dependent inhibition of doxorubicin-induced upregulation over a 72-hour period. Bars, SE. *, P<0.05, one-way ANOVA, n=3.

FIGS. 14A-14H depict doxorubicin upregulation of normoxic HIF-1α stability by activating STAT1/iNOS/NO signaling pathway.

FIG. 14A is a bar graph presenting real-time PCR results, which revealed that doxorubicin treatment significantly increased iNOS mRNA levels in 4T1ODD-luc cells in comparison to the control treatment. *, P<0.05, Student's t-test, n=3. FIG. 14B depicts Western blot analysis demonstrating that doxorubicin (1 μg/ml) stimulated iNOS expression in 4T1ODD-luc cells.

FIG. 14C depicts Western blot analysis demonstrating that doxorubicin (1 μg/ml) upregulated both the expression and phosphorylation of STAT1 (Tyr701 and Ser727) in 4T1ODD-luc cells.

FIG. 14D depicts Western blot analysis demonstrating that 1400W and EGCG suppressed the doxorubicin-induced upregulation of iNOS in 4T1ODD-luc cells.

FIG. 14E depicts Western blot analysis demonstrating that 1400W and EGCG suppressed the doxorubicin-induced upregulation of phosphorylation and expression of STAT1 in 4T1 ODD-luc cells.

FIG. 14F is a bar graph presenting the results of flow cytometry analysis of fluorescent NO indicator DAF-FM, revealing that iNOS-specific inhibitor 1400W significantly suppressed the doxorubicin-induced upregulation of intracellular NO level in 4T1ODD-luc cells. Results were normalized to the mean value of the control group. *, P<0.05, One-way ANOVA, n=3. Dox, doxorubicin.

FIG. 14G is a bar graph presenting the results of flow cytometry analysis of fluorescent NO indicator DAF-FM, revealing that STAT1-specific inhibitor EGCG significantly suppressed the doxorubicin-induced upregulation of intracellular NO level in 4T1ODD-luc cells. Results were normalized to the mean value of the control group. *, P<0.01, One-way ANOVA, n=3. Dox, doxorubicin.

FIG. 14H depicts Western blot analysis demonstrating that 1400W and EGCG suppressed the doxorubicin-induced normoxic HIF-1α accumulation in 4T1ODD-luc cells on post-treatment day 2 and in MCF-7 cells on post-treatment day 3.

FIG. 15 is a schematic diagram summarizing doxorubicin-induced normoxic HIF-1α stabilization through the activation of the STAT1/iNOS/NO signaling pathway, which can lead to resurgent angiogenesis in post-treatment tumors.

FIG. 16 is a graphical representation comparing the effects of doxorubicin and saline (control) on tumor perfusion. Perfusion dye Hoechst 33342 labeling demonstrated no significant difference in perfused tumor fraction between doxorubicin and saline and treatment. Bars, SE. P>0.05, Student's t-test, n=5.

FIG. 17 is a graphical representation comparing the tumor-activated macrophages in 4T1ODD-luc orthotopic tumors treated with doxorubicin or saline as the control. Immunohistochemical staining of CD68, a specific marker of tumor-associated macrophages, demonstrated no significant difference in the fraction of tumor-activated macrophages between doxorubicin-treated tumors and saline-treated tumors. Bars, SE. *, P>0.05, Student's t-test, n=5.

FIG. 18 is a graphical representation of the results of a cell viability assay demonstrating that combining 1400W or EGCG with doxorubicin did not attenuate the cell killing effects of doxorubicin. #, P>0.05, One-way ANOVA, n=5.

FIG. 19 is a bar graph presenting the results of C533S mutation on activated macrophage-mediated HIF-1α stabilization by use of a full-length HIF-1α-luciferase fusion reporter gene. 4T1 cells stably transduced with either the wild-type full-length HIF-Luc reporter gene or its mutant version C533S HIF-luc gene were cultured in 24-well plates to 50% confluence (1.0×10⁵ cell per well). About 1.5×10⁵ RAW 264.7 (mouse macrophage) were added after 24 hours. Two hours later, 10 μg/ml of LPS and 0.1 ng/ml of IFN-γ were added to the medium. Twelve hours later, cells were imaged for luciferase activity in the Xenogen IVIS200 system. The addition of LPS, which activated the wild type HIF-1α-luc fusion reporter significantly (p<0.005, t test), had minimal effect on the C533S mutant HIF-1α fusion reporter.

FIG. 20 is a schematic diagram summarizing radiotherapy induced HIF-1α stabilization through nitrosylation of C533 by macrophage-derived nitric oxide.

### DETAILED DESCRIPTION

### I. General Considerations

Radiotherapy and chemotherapy are two of the three main modalities of cancer therapy. However, for the majority of cancer patients, the therapeutic efficacy of chemotherapy or radiotherapy is not ideal. Many tumors are resistant to various chemotherapy and radiotherapy treatments. At the molecular level, the mechanisms involved in such resistance are not completely understood. However, recent studies indicate that hypoxia-inducible factor 1 (HIF-1) factor might be involved. These studies have shown that radiation and chemotherapy can upregulate the level and activity of HIF-1 protein and this upregulation is related to increased tumor angiogenesis and tumor resistance to therapy. As such, inhibition of HIF-1 activity can significantly increase the sensitivity of tumor cells to radiotherapy and chemotherapy.

Recent progress in the understanding of tumor physiology and the tumor microenvironment has yielded new targets that can be used to develop novel therapeutic agents. One such target is HIF-1. HIF-1 is a master transcriptional regulator that plays roles in development, physiology, and many pathological processes (Semenza et al., 2000; Semenza, 2002; Semenza, 2003; Melillo, 2004). Originally identified as a transcription factor activated under conditions of abnormally low oxygen (Wang & Semenza, 1993a; Wang & Semenza, 1993b), HIF-1's potential roles in cancer biology are a topic of current interest. More than 60 genes have been identified as direct targets of HIF-1 activity (Semenza, 2003) including, but not limited to genes involved in angiogenesis, metabolic adaptation, apoptosis induction/resistance, and invasion/metastasis.

HIF-1 is a heterodimeric protein that consists of the constitutively expressed HIF-1β subunit (also called aryl hydrocarbon receptor nuclear translocator; ARNT) and the highly regulated HIF-1α subunit (Wang & Semenza, 1995). The overall activity of HIF-1 is determined by intracellular HIF-la level. In the past decade, certain insights related to HIF-1α regulation have been realized. One significant advance has been the discovery of HIF-1α regulation by oxygen tension, which is mainly mediated by the ubiquitin-proteasome pathway. Under normoxic conditions, human HIF-1α is hydroxylated by one or more prolyl hydroxylases (PHDs) at proline residues 402 and 564 in the oxygen dependent domain (ODD; Ivan et al., 2001; Jaakkola et al., 2001). This hydroxylation renders HIF-1α susceptible to binding and ubiquitylation by E3 ubiquitin protein ligases, which contain the von Hippel-Lindau tumor suppressor protein (VHL; Pause et al., 1999; Maxwell et al., 1999; Maxwell et al., 2001). Ubiquitylated HIF-1α is then rapidly degraded by the proteasome. Under hypoxic conditions, the enzymatic activities of PHDs are significantly reduced due to the oxygen-dependent nature of PHDs. As a result, HIF-1α accumulates.

In addition to hydroxylation of the proline residues in the ODD domain by PHDs, hydroxylation of the asparagine at residue 803 in human HIF-1α, which is located in the transactivation domain, by a polypeptide termed factor inhibiting HIF-1 protein (FIH-1) has been found to regulate the activity of HIF-1α by preventing its interaction with two co-activators—p300 and CBP (Lando et al., 2002a; Lando et al., 2002b). Acetylation of a lysine residue (Lys 532 in human HIF-1α) has also been shown to regulate HIF-1 by enhancing the binding of HIF-1α to VHL and its subsequent degradation (Jeong et al., 2002). The ARD1 acetyl transferase has been shown to be responsible for this acetylation.

Still another recently identified mechanism of HIF-1 regulation is fumarate-dependent. Intracellular fumarate is regulated by fumarate hydratase, an enzyme in the tricarboxylic acid (TCA) cycle. Mutations in this gene, which occur in hereditary leiomyomatosis, were shown to cause increased levels of intracellular fumarate. The increased fumarate can act as a competitive inhibitor of prolyl hydroxylase, causing increased level of HIF-1α to accumulate (Isaacs et al., 2005). A similar function has been identified for succinate dehydrogenase (SDH), which is another member of the TCA cycle. Mutations of SDH, a candidate tumor suppressor for renal cell carcinoma, leads to increased succinate level, which has been shown to inhibit PHD activity and to lead to increased HIF-1α levels (Selak et al., 2005).

In solid tumors, HIF-1α activity is regulated via several mechanisms. Hypoxia is a common feature of all solid tumor microenvironments by virtue of the rapid proliferation of tumor cells and the generally poor functionality of newly formed tumor vasculature. Therefore, in the majority of solid tumors, hypoxia plays an important role in upregulating HIF-1α activity (Harris, 2002). In fact, hypoxia-induced HIF-1 activation and subsequent VEGF expression has been postulated to be a major driving force in tumor angiogenesis in solid tumors (Maltepe et al., 1997; Harris, 2002).

In addition to hypoxia, many hypoxia-independent pathways of HIF-1 regulation have been identified. These are mainly genetic/epigenetic alterations that can upregulate the level and/or activity of the HIF-1α polypeptide. Loss of VHL (Maxwell et al., 1999; Ohh et al., 2000) and/or p53 gene function (Ravi et al., 2000; Chen et al., 2003; Sanchez-Puig et al., 2005), which decreases the ubiquitylation and subsequent degradation of HIF-1α protein, can significantly upregulate HIF-1 activity. In addition, mutations in the PTEN tumor suppressor gene (Zundel et al., 2000; Zhong et al., 2000), which increase activity of the PI3K-AKT-mTOR signaling pathway (Laughner et al., 2001; Chan et al., 2002); ERBB2 gain of function mutations (Laughner et al., 2001); increased EGFR (Zhong et al., 2000), MEK-ERK (Fukuda et al., 2002), and/or IGF-1R signaling (Fukuda et al., 2003); and SRC gain of function mutations (Jiang et al., 1997) can all cause increased synthesis of the HIF-1α protein and overall HIF-1 activation.

HIF-1 and Radiation Therapy

In addition to tumor microenvironmental conditions and genetic/epigenetic changes in host tumor cells, it has recently been shown that HIF-1 activity can also be modified by exposure to radiotherapy (Moeller et al., 2004; Moeller et al., 2005). Exposure to ionizing radiation appears to activate HIF-1 via a hypoxia-independent mechanism. This activation appears to be mediated by a post-transcriptional mechanism that involves the release of pre-stored HIF-1α-encoding mRNAs in “stress granules” located in the cytoplasm (Moeller et al., 2004). The triggering signals were identified to be free radical species induced by exposure to ionizing radiation.

This discovery indicates that tumors respond to radiotherapy by activating HIF-1, which mediates the expression of VEGF and other factors that protect tumor vasculature against cytotoxic therapy, thereby increasing overall tumor cell survival. Consistent with this hypothesis are data indicating that combining radiotherapy with HIF-1 inhibitors appears to synergize their anti-tumor effects (Moeller et al., 2004).

Accordingly, disclosed herein is the identification of nitric oxide as a major regulator of HIF-1α activity during cancer treatment. Thus, NO inhibitors can be employed as sensitizers of cancer to cancer therapy, i.e. radiation and/or chemotherapy.

Also disclosed herein is the discovery that an important cysteine (Cys 520) residue in the human HIF-1α protein is responsible NO-mediated activation of HIF-1α during cancer therapy. This residue serves as the site for nitrosylation and subsequent activation of the HIF-1α during cancer therapy. The absence of this residue abolishes the induction of HIF-1α. Therefore, Cys 520 and corresponding residues in HIF-1α polypeptides from other species are targets for drug development.

Also disclosed herein is the discovery that compositions that can inhibit the production of nitric oxide can significantly increase therapeutic efficacy of radiotherapy through the inhibition of radiation-induced HIF-1α upregulation. Therefore, inhibitors of nitric oxide production can act as sensitizers of radiation and cancer treatment.

Also disclosed herein is the administration of agents that can inhibit the production of NO and subsequent nitrosylation and stabilization of the HIF-1α protein in tumors before, during, or after radiation therapy or cytotoxic chemotherapy. One rationale is that these agents would be expected to decrease the level of NO in the tumor microenvironment and cause a concomitant reduction of the level of HIF-1α that is induced by radiation or chemotherapy. Because HIF-1α has been shown to be a key angiogenesis regulator and survival factor for tumors during cancer therapy, the lower level of HIF-1α should allow for a better therapeutic outcome.

In addition, agents that can inhibit the nitrosylation and activation of HIF-1α either directly or indirectly can also serve as inhibitors of anti-inflammatory agents. To that end, disclosed herein is the discovery that treatment of macrophages with inflammation-causing agents can result in the stabilization and activation of HIF-1α. As HIF-1α has been shown to be important in mediating inflammatory response, agents that inhibit the nitrosylation and stabilization of HIF-1α can also be used as anti-inflammatory agents.

HIF-1 and Chemotherapy

Also disclosed herein is the discovery that HIF-1α stabilization and the formation of HIF-1 play a role in the effectiveness of current chemotherapeutic regimens. Chemotherapy is the most common systemic treatment for human tumors (Shaked et al., 2008). Although tumors may respond clinically to chemotherapy with partial or even complete remission, they often relapse with potentiated malignant behaviors such as angiogenesis and therapeutic resistance. One strategy tumor cells deploy against chemotherapy is to activate homeostatic signaling pathways to adapt, survive, and modulate the microenvironment. There is much interest in investigating these pathways so as to develop novel therapeutic strategies that block this adaptive response, thereby enhancing the effectiveness of current chemotherapeutic regimens.

High HIF-1α level can be an independent prognostic factor for poor chemotherapeutic response, early tumor relapse, and shortened survival time in many human tumors such as breast cancer. Therefore, there remains a need to identifying key molecular pathways that upregulate HIF-1α during cytotoxic therapy and the development of novel therapeutic strategies to disrupt those pathways.

In contrast to the considerable investigation into the interplay between chemotherapy and the HIF-1α stabilized by hypoxia or hypoxia-mimic chemicals, little is known about how chemotherapy regulates the stability of HIF-1α under normoxic conditions. This dearth is mainly due to the difficulty of monitoring the dynamic changes of HIF-1α expression in chemotherapy-treated normoxic tumor cells, which is caused by the impairment of the HIF-1α protein synthesis machinery in the presence of chemotherapy and as well as the rapid degradation of HIF-1α under normoxic conditions. To elucidate the effects of chemotherapy alone on HIF-1α expression in normoxic tumor cells, the presently disclosed subject matter employed a 4T1 mouse breast tumor cell line (4T1ODD-luc) which was stably transduced with a fused HIF-1α reporter gene consisting of the oxygen dependent degradation (ODD) domain of HIF-1α and a firefly luciferase gene (Li et al., 2007). By using this HIF-1α reporter cell line, the above technical obstacles were overcome and normoxic HIF-1α stabilization was successfully observed shortly after doxorubicin treatment.

The instant disclosure therefore provides results of an investigation of (1) how doxorubicin affects tumor cell HIF-1α expression in normoxia, (2) the underlying molecular mechanisms of doxorubicin-related HIF-1α accumulation and its downstream effect on tumor angiogenesis post-treatment, and (3) the therapeutic strategies to suppress the doxorubicin-related normoxic HIF-1α stabilization. This work reveals the first evidence of chemotherapy-induced HIF-1α accumulation under normoxic conditions, which is caused by the activation of STAT1/iNOS/NO signaling cascade. Revealing a normoxic tumor cell as an unrecognized target for HIF-1α inhibition during doxorubicin chemotherapy has implications for trials aimed at combining HIF-1α blocking agents with chemotherapy and, potentially, a variety of other therapies. Because HIF-1α also participates in the pathologic procedures of many other diseases such as cardiovascular disease and inflammation, the mechanism identified here can also facilitate a better understanding in these diseases as well.

Disclosed herein is the discovery that HIF-1 was upregulated following doxorubicin treatment both in vitro and in vivo. Doxorubicin induced HIF-1 upregulation in tumors in vivo was more rapid than had been previously observed with radiation or cyclophosphamide. In these prior two instances it was found that HIF-1 upregulation was tied to reoxygenation (2-3 days after treatment, with an increase in free radicals) and to macrophage infiltration and an increase in nitric oxide production by the macrophages, which typically occurs 7-10 days after treatment. In contrast, the peak in HIF-1 activation after doxorubicin treatment can occur in 3-5 days, in vivo, which is too early for macrophage infiltration. Moreover, it was demonstrated that doxorubicin treatment did not cause tumor reoxygenation, ruling out hypoxia reoxygenation injury as a cause for HIF-1 stabilization.

In addition, the HIF-1 stabilization in doxorubicin-treated tumor cells occurred under normoxic conditions and did not have macrophage co-culture system in the in vitro experiments, suggesting the mechanism of doxorubicin-induced HIF-1 upregulation is different from hypoxia-induced or macrophage-related HIF-1 stabilization. This surprising result is distinct from a previous report suggesting doxorubicin downregulates HIF-1 expression in hypoxic tumor cells and that metronomic dosing of doxorubicin can decrease HIF-1 expression in tumors, inhibiting angiogenesis (Lee et al., 2009). One consideration is that doxorubicin often poorly penetrates hypoxic tumor regions. As such, when doxorubicin is administered to patients with tumors, the effects of the drug on the aerobic subcompartment are likely to be greater than on the hypoxic compartment. Thus, in some embodiments the inhibition of HIF-1 in combination with doxorubicin treatment is likely to be more efficacious than doxorubicin alone, following any standard treatment regimen.

Also disclosed herein is the discovery of a mechanism of chemotherapy-induced activation and/or stabilization of HIF-1. Although increases in transcription factors after chemotherapy have been observed, no reports have demonstrated a chemotherapeutic effect on a regulatory pathway involving STAT-1 and HIF-1. Moreover, the presently disclosed subject matter further discloses the involvement of iNOS expression in the chemotherapeutic-mediated upregulation of HIF-1 by way of STAT-1. As such, disclosed herein is a novel STAT-1-iNOS-HIF-1 mechanism for HIF-1 upregulation in tumor cells and the corresponding methods to suppress tumor cell HIF-1 stabilization by inhibiting NOS, iNOS, STAT-1, or combinations thereof.

The discoveries of (1) HIF-1 upregulation after chemotherapy treatment can be mediated by endogenous nitric oxide in tumor cells themselves, (2) the mechanism of the STAT-1-iNOS-HIF-1 signaling pathway for tumor cell HIF-1 accumulation, and (3) the strategies and methods to suppress tumor HIF-1 stabilization by targeting STAT-1, iNOS, and NOS, during chemotherapy are provided herein. By providing a mechanism for inhibiting HIF-1 upregulation after chemotherapy the influence of HIF-1 on maintaining vasculature and cell metabolism and viability after treatment can be countered. This response can protect surviving tumor cells from ultimate destruction by chemotherapy. This regulatory pathway has several points of intervention, thus making it attractive for therapeutic approaches. As such, therapies could involve agents (natural or synthetic molecules, siRNA, shRNA, etc.) that inhibit STAT-1, iNOS, NOS, or even catalytically inactivate NO, alone or in combination, as an approach to sensitize tumors to chemotherapy. Currently there is no established standard of care that involves inhibition of this pathway or even the direct inhibition of HIF-1. Because HIF-1 accumulation and the downstream genes regulated by HIF-1 are important in many aspects of normal tissue stress adaptions and cancer biology such as, but not limited to, angiogenesis, therapeutic resistance, proliferation and apoptosis, invasion and metastasis, and energy metabolism, regulation of HIF-1 by the strategies and methods presented here can have broad impacts and applications involving cancer therapy.

### II. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a tumor cell” includes a plurality of such tumor cells, and so forth.

As used herein, the term “about,” when referring to a value or to an amount of mass, weight, time, volume, concentration, or percentage is meant to encompass variations of in some embodiments, ±20%, in some embodiments ±10%, in some embodiments ±5%, in some embodiments ±1%, and in some embodiments ±0.1% from the specified amount, as such variations are appropriate to perform the disclosed methods.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p value”. Those p values that fall below a user-defined cutoff point are regarded as significant. In some embodiments, a p value less than or equal to 0.05, in some embodiments less than 0.01, in some embodiments less than 0.005, and in some embodiments less than 0.001, are regarded as significant. Accordingly, a p value greater than or equal to 0.05 is considered not significant.

As used herein, the term “subject” refers to any organism for which application of the presently disclosed subject matter would be desirable. The subject treated in the presently disclosed subject matter in its many embodiments is desirably a human subject, although it is to be understood that the principles of the presently disclosed subject matter indicate that the presently disclosed subject matter is effective with respect to all vertebrate species, including mammals, which are intended to be included in the term “subject”. Moreover, a mammal is understood to include any mammalian species in which treatment of a tumor and/or a cancer is desirable, particularly agricultural and domestic mammalian species.

More particularly provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

The term “normoxia” refers to the condition of having a normal level of oxygen, as opposed to elevated or reduced oxygen. Similarly, the term “normoxic” refers to an oxygen state and specifically the state of having a normal oxygen concentration. The terms “normoxic” and “aerobic” can be used interchangeably in the instant disclosure. In some embodiments, a “normoxic” tumor, cancer cell or cancer tissue can refer to a tumor, cancer cell or cancer tissue having a substantially normal oxygen concentration, or substantially the same oxygen concentration as surrounding non-cancerous tissues.

The term “hypoxia” refers to the condition of having an abnormally low level of oxygen, as opposed to a normal level of oxygen, i.e. normoxia. Hypoxia also refers to a condition caused by insufficient oxygen in tissues and organs. “Hypoxic” refers to the state of having a reduced oxygen concentration. In some embodiments, a “hypoxic” tumor, cancer cell or cancer tissue can refer to a tumor, cancer cell or cancer tissue having a substantially reduced oxygen concentration as compared to a normoxic tumor, cancer cell or cancer tissue. In some embodiments, a tumor, cancer cell or cancer tissue of the presently disclosed subject matter having a pO₂ of less than 10 mm Hg is considered hypoxic. In some embodiments, a tumor, cancer cell or cancer tissue of the presently disclosed subject matter having a pO₂ of less than 9 mm Hg is considered hypoxic. In some embodiments, a tumor, cancer cell or cancer tissue of the presently disclosed subject matter having a pO₂ of less than 8 mm Hg, 7 mm Hg, 6 mm Hg, or 5 mm Hg is considered hypoxic.

The terms “small interfering RNA”, “short interfering RNA”, and “siRNA” are used interchangeably and refer to any nucleic acid molecule capable of mediating RNA interference (RNAi) or gene silencing. See e.g., Bass, 2001; Elbashir et al., 2001; and PCT International Publication Nos. WO 99/07409; WO 99/32619; WO 00/01846; WO 00/44895; WO 00/44914; WO 01/36646; WO 01/29058. A non-limiting example of an siRNA molecule of the presently disclosed subject matter is shown in SEQ ID NO: 17. In some embodiments, the siRNA comprises a double stranded polynucleotide molecule comprising complementary sense and antisense regions, wherein the antisense region comprises a sequence complementary to a region of a target nucleic acid molecule (for example, an mRNA encoding VHL). In some embodiments, the siRNA comprises a single stranded polynucleotide having self-complementary sense and antisense regions, wherein the antisense region comprises a sequence complementary to a region of a target nucleic acid molecule. In some embodiments, the siRNA comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. As used herein, siRNA molecules need not be limited to those molecules containing only RNA, but further encompass chemically modified nucleotides and non-nucleotides.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell (for example, the interaction between HIF-1α and VHL) can also affect gene expression as defined herein.

As used herein, the term “modulate” refers to a change in the expression level of a gene, or a level of RNA molecule or equivalent RNA molecules encoding one or more proteins or protein subunits, or activity of one or more proteins or protein subunits is upregulated or downregulated, such that expression, level, and/or activity is greater than or less than that observed in the absence of the modulator. For example, the term “modulate” can mean “inhibit” or “suppress”, but the use of the word “modulate” is not limited to this definition.

As used herein, the terms “inhibit”, “suppress”, “downregulate”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. In some embodiments, inhibition results in an expression level of a gene product that is below that level observed in the absence of the modulator.

In some embodiments, the terms “inhibit”, “suppress”, “downregulate”, and grammatical variants thereof refer to a biological activity of a polypeptide or polypeptide complex that is lower in the presence of a modulator than that which occurs in the absence of the modulator. For example, a modulator can inhibit the ability of a polypeptide (e.g., an HIF-1 polypeptide) to interact with its target (e.g., VHL and/or a promoter sequence comprising a hypoxia response element (HRE)). This can be accomplished by any mechanism, including but not limited to enhancing its existence in an inactive form (e.g., enhancing the complexing of an HIF-1 with VHL and/or inhibiting the dissociation of an HIF-1 from VHL) and/or by enhancing the rate of degradation of an HIF-1.

As used herein, the terms “gene” and “target gene” refer to a nucleic acid that encodes an RNA, for example, nucleic acid sequences including, but not limited to, structural genes encoding a polypeptide. The target gene can be a gene derived from a cell, an endogenous gene, a transgene, etc. The cell containing the target gene can be derived from or contained in any organism, for example an animal. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

In some embodiments, a gene is a hypoxia-inducible gene. As used herein, a “hypoxia-inducible gene” is a gene for which the expression level increases in response to hypoxia. In some embodiments, a hypoxia-inducible gene is a gene that is characterized by upregulated transcription in response to hypoxic conditions. Exemplary hypoxia-inducible genes thus include genes with hypoxia response elements (HREs) in their promoters. Under hypoxic conditions, transcription of these genes is induced as a result of activated HIF-1 binding to the HREs. Also as used herein, a hypoxia-inducible gene is a gene for which an activity of the gene product changes in response to hypoxia. In these embodiments, a hypoxia-inducible gene is a gene for which the polypeptide encoded by the gene experiences a change in state in response to hypoxia. Such a change in state includes, but is not limited to a post-transcriptional modification or an interaction with another molecule (for example, a protein-protein interaction). Thus, as used herein, the term hypoxia-inducible gene includes, but is not limited to HIF-1α and VHL, each of which undergoes a change in state (in this example, a dissociation one from the other) in response to hypoxia.

As is understood in the art, a gene comprises a coding strand and a non-coding strand. As used herein, the terms “coding strand” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

As used herein, the terms “complementarity” and “complementary” refer to a nucleic acid that can form one or more hydrogen bonds with another nucleic acid sequence by either traditional Watson-Crick or other non-traditional types of interactions.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence.

As used herein, the term “cell” is used in its usual biological sense. In some embodiments, the cell is present in an organism, for example, mammals such as humans, cows, sheep, apes, monkeys, swine, dogs, cats, and rodents. In some embodiments, the cell is a eukaryotic cell (e.g., a mammalian cell, such as a human cell). The cell can be of somatic or germ line origin, totipotent or pluripotent, dividing or non-dividing. The cell can also be derived from or can comprise a gamete or embryo, a stem cell, or a fully differentiated cell.

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs.

As used herein, the terms “corresponds to”, “corresponding to”, and grammatical variants thereof refer to a nucleotide sequence that is 100% identical to at least 19 contiguous nucleotides of a nucleic acid sequence of a hypoxia-inducible gene. Thus, a first nucleic acid sequence that “corresponds to” a coding strand of a hypoxia-inducible gene is a nucleic acid sequence that is 100% identical to at least 19 contiguous nucleotides of a hypoxia-inducible gene, including, but not limited to 5′ untranslated sequences, exon sequences, intron sequences, and 3′ untranslated sequences.

The terms “tumor”, “tumor cell”, “cancer”, “cancer cell”, and “cancer tissue” are used interchangeable herein and encompasses both primary and metastasized solid tumors and carcinomas of any tissue in a subject, including, but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. The term “tumor” also encompasses radioresistant and/or chemoresistant tumors, including, but not limited to radioresistant and/or chemoresistant variants of the any of the tumor listed above.

The terms “radiosensitivity” and “radiosensitive”, as used herein to describe a tumor, refer to a quality of susceptibility to treatment using ionizing radiation. Thus, radiotherapy can be used to delay growth of a radiosensitive tumor. Radiosensitivity can be quantified by determining a minimal amount of ionizing radiation that can be used to delay tumor growth. Thus, the term “radiosensitivity” refers to a quantitative range of radiation susceptibility.

The terms “sensitivity to chemotherapy”, “chemosensitivity”, “sensitivity” and “chemosensitive”, as used herein to describe a tumor and/or in reference to a tumor or cancer cell, refer to a quality of susceptibility of the tumor or cancer to treatment using chemotherapy. Thus, chemotherapy can be used to delay growth of a tumor or cancer sensitive to chemotherapy. Sensitivity of chemotherapy can be quantified by determining a minimal dosage of chemotherapy that can be used to delay tumor growth. Thus, the phrase “sensitivity to chemotherapy” refers to a quantitative range of chemotherapy susceptibility.

The terms “radiation resistant tumor” and “radioresistant tumor” each generally refer to a tumor that is substantially unresponsive to radiotherapy when compared to other tumors. Representative radiation resistant tumor models include glioblastoma multiforme and melanoma. Similarly, the terms “chemotherapy resistant tumor” and “chemoresistant tumor” generally refer to a tumor that is substantially unresponsive to chemotherapy when compared to other tumors.

The term “delaying tumor growth” refers to a decrease in duration of time required for a tumor to grow a specified amount. For example, treatment with the compositions and/or methods disclosed herein can delay the time required for a tumor to increase in volume 3-fold relative to an initial day of measurement (day 0) or the time required to grow to 1 cm³.

The term “increase,” as used herein to refer to a change in radiosensitivity and/or sensitivity to chemotherapy of a tumor, refers to change that renders a tumor more susceptible to destruction by ionizing radiation and/or chemotherapy. Alternatively stated, an increase in radiosensitivity and/or chemosensitivity refers to a decrease in the minimal amount of ionizing radiation and/or chemotherapy that effectively delays tumor growth. An increase in radiosensitivity and/or chemosensitivity can also comprise delayed tumor growth when a composition of the presently disclosed subject matter is administered with radiation and/or chemotherapy as compared to a same dose of radiation and/or chemotherapy alone. In some embodiments, an increase in radiosensitivity and/or chemosensitivity refers to an increase of at least about 2-fold, in some embodiments an increase of at least about 5-fold, and in some embodiments an increase of at least 10-fold. In some embodiments of the presently disclosed subject matter, an increase in radiosensitivity and/or chemosensitivity comprises a transformation of a radioresistant and/or chemoresistant tumor to a radiosensitive and/or chemosensitive tumor.

The term “tumor regression” generally refers to any one of a number of indices that suggest change within the tumor to a less developed form. Such indices include, but are not limited to a destruction of tumor vasculature (for example, a decrease in vascular length density or a decrease in blood flow), a decrease in tumor cell survival, a decrease in tumor volume, and/or a decrease in tumor growth rate. Methods for assessing tumor growth delay and tumor regression are known to the skilled artisan.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses complementary sequences, subsequences, elongated sequences, as well as the sequence explicitly indicated. The terms “nucleic acid molecule” or “nucleotide sequence” can also be used in place of “gene”, “DNA”, “cDNA”, “RNA”, or “mRNA”. Nucleic acids can be derived from any source, including any organism.

The term “isolated”, as used in the context of a nucleic acid molecule or polypeptide, indicates that the nucleic acid molecule or polypeptide exists apart from its native environment and is not a product of nature. An isolated nucleic acid molecule or polypeptide can exist in a purified form or can exist in a non-native environment such as a host cell.

The terms “identical” or percent “identity” in the context of two or more nucleotide or polypeptide sequences refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical”, in the context of two nucleotide sequences, refers to two or more sequences or subsequences that have in some embodiments at least 60%, in some embodiments about 70%, in some embodiments about 80%, in some embodiments about 90%, in some embodiments about 95%, in some embodiments about 96%, in some embodiments about 97%, in some embodiments about 98%, and in some embodiments about 99% nucleotide identity, when compared and aligned for maximum correspondence, as measured using one of the following sequence comparison algorithms (described herein below) or by visual inspection. In some embodiments, the substantial identity exists in nucleotide sequences of at least 50 residues, in some embodiments in nucleotide sequence of at least about 100 residues, in some embodiments in nucleotide sequences of at least about 150 residues, and in some embodiments in nucleotide sequences comprising complete coding sequences.

In one aspect, polymorphic sequences can be substantially identical sequences. The terms “polymorphic”, “polymorphism”, and “polymorphic variants” refer to the occurrence of two or more genetically determined alternative sequences or alleles in a population. An allelic difference can be as small as one base pair. As used herein in regards to a nucleotide or polypeptide sequence, the term “substantially identical” also refers to a particular sequence that varies from another sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman, 1981, by the homology alignment algorithm of Needleman & Wunsch, 1970, by the search for similarity method for Pearson & Lipman, 1988, by computerized implementations of these algorithms (GAP, BESTFIT, FASTA, and TFASTA, in the Wisconsin Genetics Software Package, available from Accelrys Inc., San Diego, Calif., United States of America), or by visual inspection. See generally, Ausubel, 1995.

In some embodiments, an algorithm for determining percent sequence identity and sequence similarity is the BLAST algorithm, which is described by Altschul et al., 1990. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of highly stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× standard saline citrate (SSC), 0.1% (w/v) SDS at 65° C. Another example of highly stringent wash conditions is 15 minutes in 0.2×SSC buffer at 65° C. (see Sambrook & Russell, 2001 for a description of SSC buffer and other stringency conditions). Often, a high stringency wash is preceded by a lower stringency wash to remove background probe signal. An example of medium stringency wash conditions for a duplex of more than about 100 nucleotides is 15 minutes in 1×SSC at 45° C. Another example of medium stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold or higher than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm EDTA at 50° C. followed by washing in 2×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 1×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.5×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.1×SSC, 0.1% SDS at 50° C.; in yet another example, a probe and target sequence hybridize in 7% sodium dodecyl sulfate (SDS), 0.5M NaPO₄, 1 mm EDTA at 50° C. followed by washing in 0.1×SSC, 0.1% SDS at 65° C.

The term “subsequence” refers to a sequence of a nucleic acid or polypeptide that comprises a part of a longer nucleic acid or polypeptide sequence.

The term “elongated sequence” refers to an addition of nucleotides (or other analogous molecules) or amino acid residues incorporated into the nucleic acid or polypeptide. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The terms “operatively linked” and “operably linked”, as used herein, refer to a nucleic acid molecule in which a promoter region is connected to a nucleotide sequence in such a way that the transcription of that nucleotide sequence is controlled and regulated by the promoter region. Similarly, a nucleotide sequence is said to be under the “transcriptional control” of a promoter to which it is operably linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell and/or, if from the same source, is modified from its original form. Thus, a heterologous gene in a host cell includes a gene that is endogenous to the particular host cell but has been modified, for example by mutagenesis and/or by isolation from native transcriptional regulatory sequences. The terms also include non-naturally occurring multiple copies of a naturally occurring nucleotide sequence. Thus, the terms refer in some embodiments to a DNA segment that is foreign or heterologous to the cell, or is homologous to the cell but in a position within the host cell nucleic acid wherein the element is not ordinarily found.

The term “expression vector” as used herein refers to a nucleotide sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

The term “promoter” or “promoter region” each refers to a nucleotide sequence within a gene that is positioned 5′ to a coding sequence and functions to direct transcription of the coding sequence. The promoter region comprises a transcriptional start site, and can additionally include one or more transcriptional regulatory elements. In some embodiments, a method for the presently disclosed subject matter employs a hypoxia inducible promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human β-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al., 1991), adenosine deaminase, phosphoglycerate kinase (PGK), pyruvate kinase, phosphoglycerate mutase, the β-actin promoter (see e.g., Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the PSA promoter (Yu et al., 1999; Lee et al., 2000), the probasin promoter (Greenberg et al., 1994; Yu et al., 1999), and the MUC1 promoter (Kurihara et al., 2000) as discussed above, as well as other tissue-specific and cell-type specific promoters known to those of skill in the art.

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element.

The term “transcription factor” generally refers to a protein that modulates gene expression by interaction with the transcriptional regulatory element and cellular components for transcription, including RNA polymerase, Transcription Associated Factors (TAFs), chromatin-remodeling proteins, and any other relevant protein that impacts gene transcription.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook, 1990 and PCT International Publication No. WO 97/47763. Exemplary reporter genes for transcriptional analyses include the lacZ gene (see e.g., Rose & Botstein, 1983), Green Fluorescent Protein (GFP; Cubitt et al., 1995), luciferase, and chloramphenicol acetyl transferase (CAT). Reporter genes for methods to produce transgenic animals include but are not limited to antibiotic resistance genes, for example the antibiotic resistance gene confers neomycin resistance. Any suitable reporter and detection method can be used, and it will be appreciated by one of skill in the art that no particular choice is essential to or a limitation of the presently disclosed subject matter.

An amount of reporter gene can be assayed by any method for qualitatively or quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, in one example an increase by about 2-fold or greater relative to a control measurement, in another example an increase by about 5-fold or greater, and in yet another example an increase by about 10-fold or greater.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984; Ausubel et al., 1992; Glover & Hames, 1995; and Sambrook & Russell, 2001). Site-specific mutagenesis to create base pair changes, deletions, or small insertions is also known in the art as exemplified by publications (see e.g., Adelman et al., 1983; Sambrook & Russell, 2001).

### III. COMPOSITIONS

The compositions disclosed herein can be employed in vitro and/or in vivo in order to perform the disclosed methods. In some embodiments, the compositions described herein comprise an agent selected from the group consisting of an inhibitor of nitric oxide synthase, a nitric oxide scavenger, an inhibitor of NIF-1 nitrosylation, a STAT1 inhibitor, and combinations thereof.

The terms “inhibitor of HIF-1 activity”, “inhibitor of HIF-1” and “HIF-1 inhibitor” are used interchangeably throughout the instant disclosure and refer to any compound or molecule that directly or indirectly inhibits, reduces or attenuates the expression, concentration, activity or effectiveness of HIF-1. An inhibitor of HIF-1 activity as used herein can act directly on HIF-1, or can indirectly impact HIF-1 by acting on an intermediate in a pathway involving HIF-1 (see, e.g. FIG. 15). By way of example and not limitation, an inhibitor of HIF-1 activity can comprise an inhibitor of STAT1, NOS, iNOS or a NO scavenger, details of which are discussed further hereinbelow.

III.A. Inhibitors of Nitric Oxide Synthase(s)

In some embodiments, an agent comprises an inhibitor of nitric oxide synthase (NOS). As is known in the art, there are several nitric oxide synthases, including but not limited to neural/neuronal NOS (nNOS; also referred to as NOS1), inducible NOS (iNOS; also referred to as NOS2), and endothelial NOS (eNOS; also referred to as NOS3). Nucleic acid and amino acid sequences for each of these NOS gene products are present in the GENBANK® database, each of which is expressly incorporated by reference herein in its entirety. For example, human NOS sequences present in the GENBANK® database include GENBANK® Accession Nos. U17327 and AAA62405 (nNOS nucleic acid and amino acid sequences, respectively), NM_{—}000625 and NP_{—}000616 (iNOS nucleic acid and amino acid sequences, respectively), and BC069465 and AAH69465 (eNOS nucleic acid and amino acid sequences, respectively).

Various inhibitors of NOS have been identified, some of which are selective and other of which are non-selective for one or more specific NOS type. As used herein, a “selective” or “specific” NOS inhibitor demonstrates markedly greater specificity for one of the NOS types (e.g., iNOS) than it does for the other two (e.g., eNOS and nNOS), while “non-selective” or “non-specific” NOS inhibitors demonstrate approximately equivalent inhibition of two or more of the NOS types. In some embodiments, both non-selective and selective NOS inhibitors are appropriate for use in the methods and compositions for the presently disclosed subject matter. In some embodiments, selective NOS inhibitors are appropriate for use in the methods and compositions for the presently disclosed subject matter. Representative NOS inhibitors thus include, but are not limited to L-N(6)-(1-iminoethyl)lysine tetrazole-amide (SC-51); aminoguanidine (AG); guanidinoethyldisulfide; L-NG-nitroarginine methyl ester; mercaptoethylguanidine (MEG); N^{ω}-nitro-L-arginine methyl ester (L-NAME); N-(3-(aminomethyl)benzyl)acetamidine (1400W); N^{G}-monomethyl-L-arginine (L-NMMA); 7-nitroindazole (7-NI), L-NIL(N⁶-(1-iminoethyl)-lysine (L-NIL); and N⁵-(1-iminoethyl)-L-ornithine (L-NIO); as well as their pharmaceutically acceptable salts and other derivatives.

III.B. Nitric Oxide Scavengers

In some embodiments, a composition of the presently disclosed subject matter comprises a nitric oxide (NO) scavenger. As used herein, the phrase “nitric oxide scavenger” refers to a molecule that binds to nitric oxide or otherwise makes the nitric oxide less available to take part in a biochemical process within a cell. Nitric oxide scavengers are also known, and include, but are not limited to vitamin B12, particularly in the hydroxocobalamin form; 2-(4-carboxyphenyl)-4,4,5,5-tetramethylimidazoline-1-oxyl-3-oxide (carboxy-PTIO); diethyldithiocarbamate; AMD6221 (ruthenium[hydrogen(diethylenetrinitrilo) pentaacetato]chloride); and N-dithiocarboxy-sarcosine (DTCS), as well as pharmaceutically acceptable salts and other derivatives thereof.

III.C. Inhibitors of HIF-1 Nitrosvlation

In some embodiments, a composition of the presently disclosed subject matter comprises an inhibitor of HIF-1 nitrosylation. As used herein, the phrase “inhibitor of HIF-1 nitrosylation” refers to any molecule that inhibits, either completely or partially, nitrosylation of an HIF-1 polypeptide. As such, this phrase encompasses NOS inhibitors, NO scavengers, and any other molecule that can inhibit the nitrosylation of an HIF-1 polypeptide. Representative other molecules include, but are not limited to peptides, peptide mimetics, proteins, antibodies or fragments thereof, small molecules, nucleic acids, and combinations thereof. The term “small molecule” as used herein refers to a compound, for example an organic compound, with a molecular weight in one example of less than about 1,000 daltons, in another example less than about 750 daltons, in another example less than about 600 daltons, and in yet another example less than about 500 daltons. A small molecule also has in one example a computed log octanol-water partition coefficient in the range of about −4 to about +14, more preferably in the range of about −2 to about +7.5.

As is known in the art, polypeptides such as HIF-1 can be S-nitrosylated on cysteine residues. As disclosed herein, nitrosylation of C533 of murine HIF-1α (which corresponds to C520 of human HIF-1α) interferes with the interaction between HIF-1α and VHL, which in turn inhibits the ubiquitylation and subsequent degradation of HIF-1α by the proteasome. S-nitrosylation of HIF-1α at this highly conserved cysteine (see also SEQ ID NOs. 1-21 showing the conservation of this cysteine in various animal species) thus results in an increased persistence of HIF-1α in the cell, leading to higher HIF-1α activity. Accordingly, inhibitors of HIF-1α nitrosylation can be employed to reduce HIF-1 activity in cells.

III.D STAT1 Inhibitors

In some embodiments, a composition of the presently disclosed subject matter comprises an inhibitor of signal transducer and activator of transcription 1 (STAT1). STAT1 is a modulator which potentiates chemotherapy-induced apoptosis (Thomas et al., 2004). It is also an upstream transcription activator for iNOS expression (Samardzic et al., 2001; Guo et al., 2008). As used herein, the phrase “STAT1 inhibitor” refers to any molecule that inhibits, suppresses, or attenuates, either completely or partially, the expression, activity or efficacy of STAT1. By way of example and not limitation, a STAT1 inhibitor of the presently disclosed subject matter can comprise epigallocatechin gallate (EGCG), and related compounds and derivatives. Other STAT1 inhibitors of the presently disclosed subject matter can comprise rosmarinic acid, salvianolic acid A, salvianolic acid B, caftaric acid, cichoric acid, chlorogenic acid, cynarin (Sped et al., 2009), green tea extract (GTE), St. John's Wort extract, fludarabine, decoy oligonucleotide (ODN), taxifolin, AG490, captopril, candesartan, myricetin, delphinidin (Alessandra et al., 2005), SSI-1 (Naka et al., 1997), ISS610, ISS834, ISS835, ISS836, ISS838, ISS839, ISS840, ISS841, ISS843, ISS845, ISS119, ISS115 (Gunning et al., 2007), PIAS1 (Liu et al., 1998) or combinations thereof. Other STAT1 inhibitors know to those of ordinary skill in the art can be employed without departing from the scope of the presently disclosed subject matter.

III.E. Formulations

The compositions of the presently disclosed subject matter comprise in some embodiments a composition that includes a pharmaceutically acceptable carrier. Any suitable pharmaceutical formulation can be used to prepare the compositions of the presently disclosed subject matter for administration to a subject.

For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above the formulations of this presently disclosed subject matter can include other agents conventional in the art having regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, the cytokines IFN-α, IFN-γ, IL2, IL4, IL6, TNF, or other cytokine affecting immune cells. In accordance with this aspect of the presently disclosed subject matter, the disclosed nucleic acid molecules can be administered in combination therapy with one or more of these cytokines.

III.F. Administration

Administration of the compositions of the presently disclosed subject matter can be by any method known to one of ordinary skill in the art, including, but not limited to intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation. In some embodiments, suitable methods for administration of a composition of the presently disclosed subject matter include, but are not limited to intravenous or intratumoral injection. Alternatively, a composition can be deposited at a site in need of treatment in any other manner, for example by spraying a composition comprising a composition within the pulmonary pathways. The particular mode of administering a composition of the presently disclosed subject matter depends on various factors, including the distribution and abundance of cells to be treated, whether a vector is employed, additional tissue- or cell-targeting features of the vector and/or composition, and mechanisms for metabolism or removal of the composition from its site of administration. For example, relatively superficial tumors can be injected intratumorally. By contrast, internal tumors can be treated by intravenous injection.

In some embodiments, the method for administration encompasses features for regionalized delivery or accumulation at the site in need of treatment. In some embodiments, a composition is delivered intratumorally. In some embodiments, selective delivery of a composition to a tumor is accomplished by intravenous injection of the composition.

Alternatively or in addition, a composition of the presently disclosed subject matter can be provided at a pre-determined site using an implantable device containing the composition, whereby longer term delivery of the composition to a target tissue can be accomplished. Representative implantable devices are known in the art. For example, absorbable thermoplastic elastomers have been developed to address the need in medical device development for an elastic material (e.g., U.S. Pat. Nos. 5,468,253 and 5,713,920). In addition, absorbable polymeric liquids and pastes have been developed to increase the range of physical properties exhibited by the aliphatic polyesters based on glycolide, lactide, p-dioxanone, 5,5-dimethyl-1,3-dioxan-2-one, trimethylene carbonate, and ε-caprolactone (e.g., U.S. Pat. Nos. 5,411,554; 5,599,852; 5,631,015; 5,653,992; 5,688,900; 5,728,752; and 5,824,333).

U.S. Pat. Nos. 5,573,934 and 5,858,746 (both to Hubbell et al.) disclosed the use of photocurable polymers to encapsulate biological materials including drugs, proteins, and cells in a hydrogel. The hydrogel was formed from a water soluble biocompatible macromer containing at least two free radical polymerizable substituents and either a thermal or light activated free radical initiator. An example of such a photoreactive system is an acrylate ester endcapped poly(ethylene glycol) containing ethyl eosin and a tertiary amine. After a series of light activated reactions between ethyl eosin and the amine, the acrylate endgroups polymerize into short segments that result in a crosslinked polymeric network composed of poly(ethylene glycol) chains radiating outward from the acrylate oligomers. The physical and mechanical properties of the resulting hydrogel are dependent on the reproducibility of the free radical oligomerization reaction.

U.S. Pat. No. 5,410,016 in the form of photocurable, segmented block copolymers composed not only of water soluble segments, such as poly(ethylene glycol), but also of segments with hydrolizable groups, in particular, with short segments of aliphatic polyesters. In this way, the resulting hydrogel breaks down into soluble units in vitro and in vivo in a controlled fashion. The photochemistry is the same and based on the free radical polymerization of acrylate and methacrylate endgroups.

Other implantable devices are described in U.S. Pat. Nos. 7,009,034; 7,011,842; and 7,012,126.

For delivery of compositions to pulmonary pathways, the presently disclosed subject matter can be formulated as an aerosol or coarse spray. Methods for preparation and administration of aerosol or spray formulations can be found, for example, in Cipolla et al., 2000, and in U.S. Pat. Nos. 5,858,784; 6,013,638; 6,022,737; and 6,136,295.

III.G. Dosage

An effective dose of a composition of the presently disclosed subject matter is administered to a subject in need thereof. A “therapeutically effective amount” is an amount of the composition sufficient to produce a measurable response (e.g., a cytolytic response in a subject being treated). In some embodiments, an activity that inhibits tumor growth is measured. In some embodiments, the expression and/or activity of HIF-1, or relative decrease thereof, is measured. Actual dosage levels of active ingredients in the compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, and the condition and prior medical history of the subject being treated. However, it is within the skill of the art to start doses of the compositions at levels lower than required to achieve the desired therapeutic effect and to gradually increase the dosage until the desired effect is achieved.

The potency of a composition can vary, and therefore a “therapeutically effective” amount can vary. However, one skilled in the art can readily assess the potency and efficacy of a candidate composition of the presently disclosed subject matter and adjust the therapeutic regimen accordingly.

After review of the disclosure of the presently disclosed subject matter presented herein, one of ordinary skill in the art can tailor the dosages to an individual patient, taking into account the particular formulation, method for administration to be used with the composition, and tumor type, location and size. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

For example, for local administration of viral expression vectors, previous clinical studies have demonstrated that up to 10¹³ plaque-forming units (pfu) of virus can be injected with minimal toxicity. In human patients, 1×10⁹-1×10¹³ pfu are routinely used (see Habib et al., 1999). To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor or anti-angiogenic activity. Representative criteria and methods for assessing anti-tumor and/or anti-angiogenic activity are described herein below. With replicative virus vectors, a dosage of about 1×10⁷ to 1×10⁸ pfu can be used in some instances.

### IV. APPLICATIONS

The presently disclosed subject matter provides methods for inhibiting nitric oxide synthase(s) activity and/or HIF-1 activity in a cell, cancer tissue or tumor in vitro and/or in vivo. In some embodiments, the methods comprise administering to the cell, in some embodiments in a subject, a composition comprising an agent selected from the group consisting of (i) an inhibitor of nitric oxide synthase; (ii) a nitric oxide scavenger; (iii) an inhibitor of HIF-1 nitrosylation; (iv) an inhibitor of STAT1; and (v) combinations thereof, whereby nitric oxide synthase activity and/or HIF-1 activity in the cell is inhibited. This general strategy can be employed in several areas, as disclosed in more detail hereinbelow.

IV.A. Methods for Inhibiting HIF-1 Activity

In some embodiments, the presently disclosed subject matter provides methods and compositions for inhibiting HIF-1 activity in a cell. In some embodiments, the cell is a tumor cell, and in some embodiments, the tumor cell is present within a subject including, but not limited to a mammals such as a human.

As disclosed herein, the presently disclosed subject matter provides compositions for inhibiting HIF-1 activity in a cell by inhibiting nitrosylation of HIF-1α, which in turn results in enhanced degradation of HIF-1α mediated by the ubiquitin-proteasome pathway. As also disclosed herein, a highly conserved cysteine residue has been found to be a site for nitrosylation. This highly conserved cysteine corresponds to the positions listed in Table 1.

TABLE 1
Conserved Cysteines in HIF-1α from Various Species

| Species | Cys Position |
|---|---|
| Spalax judaei | 520 of SEQ ID NO: 1 |
| Eospalax baileyi | 521 of SEQ ID NO: 2 |
| Mus musculus | 533 of SEQ ID NO: 3 |
| Rattus norvegicus | 520 of SEQ ID NO: 4 |
| Microtus oeconomus | 520 of SEQ ID NO: 5 |
| Homo sapiens | 520 of SEQ ID NO: 6 |
| Pongo pygmaeus | 521 of SEQ ID NO: 7 |
| Macaca fascicularis | 520 of SEQ ID NO: 8 |
| Spermophilus tridecemlineatus | 520 of SEQ ID NO: 9 |
| Bos taurus | 520 of SEQ ID NO: 10 |
| Pantholops hodgsonii | 520 of SEQ ID NO: 11 |
| Canis familiaris | 520 of SEQ ID NO: 12 |
| Oryctolagus cuniculus | 520 of SEQ ID NO: 13 |
| Gallus gallus | 518 of SEQ ID NO: 14 |
| Xenopus laevis | 516 of SEQ ID NO: 16 |

Accordingly, the methods and compositions disclosed herein inhibit HIF-1 activity in some embodiments by inhibiting nitrosylation of the listed cysteine residues.

IV.B. Methods for Treating Tumor Cells and/or Cancer Cells

The presently disclosed methods and compositions can also be employed for treatment of tumor cells and/or cancer cells. In some embodiments, the methods comprise contacting the tumor cell and/or the cancer cell with the presently disclosed compositions (e.g., by administering the compositions to a subject that has the tumor cell and/or the cancer cell). In some embodiments, the compositions are selected from the group consisting of inhibitors of nitric oxide synthase, nitric oxide scavengers, inhibitors of HIF-1 nitrosylation, STAT1 inhibitors, and combinations thereof. In some embodiments, the methods and compositions disclosed herein treat the tumor cell and/or the cancer cell by inhibiting HIF-1 activity in the tumor cell and/or the cancer cell.

As such, the methods and compositions can act directly on the tumor cell and/or the cancer cell to modulate its growth and/or proliferation. However, the methods and compositions can also act indirectly on the tumor cell and/or the cancer cell to modulate its growth and/or proliferation by inhibiting HIF-1 activity in other cells that influence the growth and/or proliferation of the tumor cell and/or the cancer cell. For example, the methods and compositions can inhibit HIF-1 activity in tumor blood vessels (i.e., those blood vessels and other endothelial cells that provide nutrients and remove waste products from the tumor and/or cancer cells) and/or can inhibit tumor angiogenesis modulated by HIF-1 activity. While applicants do not wish to be bound by any particular theory of operation, the methods and compositions disclosed herein can be employed to interfere with the function and/or generation of tumor vasculature, thereby modulating tumor cell and/or cancer cell growth and/or proliferation.

Additionally, the methods and compositions disclosed herein can be employed for increasing the sensitivity of a tumor cell and/or a cancer cell to a treatment, such as surgical resection, radiotherapy, and/or chemotherapy, as discussed in more detail hereinbelow. As used herein, the phrase “increasing the sensitivity of a tumor cell and/or a cancer cell to a treatment” refers to an enhancement of the effect that a combination treatment including use of the presently disclosed methods and compositions has on tumor cell and/or cancer cell growth and/or proliferation as compared to the effect that a treatment would have had under the same conditions absent use of the presently disclosed methods and compositions. In some embodiments, the combination treatment employs the methods and/or compositions disclosed herein in conjunction with surgical resection, radiotherapy, and/or chemotherapy, and in some embodiments, the inclusion of a treatment comprising the methods and/or compositions disclosed herein results in a synergistic (i.e., more than additive) effect. Given the current limitations of surgical resection, radiotherapy, and/or chemotherapy, the presently disclosed subject matter provides an additional therapy that can be used to increase the efficacy of medical treatments directed towards modulating tumor cell and/or cancer cell growth and proliferation.

To increase the sensitivity of a tumor cell and/or a cancer cell to a treatment, such as surgical resection, radiotherapy, and/or chemotherapy, the tumor cell and/or cancer cell can be contacted with a composition of the presently disclosed subject matter before, during, and/or after surgical resection, radiotherapy, and/or chemotherapy. In some embodiments, a tumor cell and/or cancer cell can be contacted with a composition of the presently disclosed subject matter substantially simultaneously with the administration of radiation and/or chemotherapy, or shortly after administration of radiation and/or chemotherapy. In some embodiments, a tumor cell and/or cancer cell can be contacted with a composition of the presently disclosed subject matter within a 1, 2, 3, 4, 5, 6, 7, 8, 9, 10, 15, 20 or 30 day period, i.e. before or after, of administrating radiation and/or chemotherapy.

IV.C. Methods for Inhibiting an Inflammatory Response

The methods and compositions disclosed herein can also be employed for inhibiting inflammatory responses of cells (i.e., a cell in a subject). Also disclosed herein is the discovery that treatment of macrophages with inflammation-causing agents can result in the stabilization and activation of HIF-1α. As HIF-1α has been shown to be important in mediating inflammatory response, methods and compositions that inhibit the nitrosylation and stabilization of HIF-1α can also be used as anti-inflammatory agents.

IV.D. Methods and Compositions for Identifying New Nitrosylation Inhibitors

The presently disclosed subject matter also provides screening methods and compositions that can be employed for identifying potential inhibitors of nitrosylation of an HIF-1 polypeptide. In some embodiments, the methods comprise (a) providing a cell comprising a nucleic acid a nucleotide sequence comprising any of SEQ ID NOs: 18-21; (b) contacting the cell with a compound comprising a potential inhibitor of nitrosylation of an HIF-1 polypeptide; and (c) assaying nitrosylation of a cysteine residue present in the nucleic acid, whereby an inhibitor of nitrosylation of an HIF-1 polypeptide is identified. In some embodiments, the methods further comprise comparing a level of nitrosylation of the cysteine residue present in the nucleic acid to a level of nitrosylation of the cysteine residue present in the nucleic acid prior to the contacting step.

In some embodiments, the nucleic acid comprises an expression vector in which the nucleic acid is operably linked to a promoter that is active in the cell. In some embodiments, the expression vector is a transgene and the animal is a transgenic animal that expresses the nucleic acid. In some embodiments, the compound is administered to the transgenic animal via a route that results in the compound contacting the cell. In some embodiments, the cell is an in vitro cultured cell and the contacting is performed in vitro.

In some embodiments, the cell (e.g., an in vitro cultured cell or a cell in a transgenic animal) comprises an expression construct comprising one or more of SEQ ID NOs: 18-21 operably linked to a promoter. In some embodiments, the cell (e.g., an in vitro cultured cell or a cell in a transgenic animal) comprises an expression construct comprising one or more of SEQ ID NOs: 18-21 operably linked to a promoter, with the proviso that all cysteine residues present within SEQ ID NOs: 18-21 have been replaced with a non-nitrosylatable amino acid. An exemplary non-nitrolysable amino acid is serine.

The cells comprising the expression construct comprising one or more of SEQ ID NOs: 18-21 operably linked to a promoter (e.g., an in vitro cultured cell or a cell in a transgenic animal) can be employed for screening candidate compounds for an ability to modulate nitrosylation of HIF-1. As used herein, the terms “candidate substance” and “candidate compound” are used interchangeably and refer to a substance that is believed to be capable of modulating nitrosylation of the conserved cysteine present in any of SEQ ID NOs: 18-21. Exemplary candidate compounds that can be investigated using the methods and compositions disclosed herein include, but are not restricted to, agonists and antagonists of enzymes disclosed herein to influence HIF-1 nitrosylation (e.g., small molecule agonists and antagonists of a NOS and/or a PDH), peptides, small molecules, and antibodies and derivatives thereof, and combinations thereof.

Assays that can be employed for screening a candidate compound for an ability to modulate HIF-1 nitrosylation are known in the art, and include, but are not limited to the “biotin switch” experiment described in Jaffrey & Snyder, 2001, and in EXAMPLE 6.

### V. COMBINATION THERAPY

The presently disclosed subject matter can be employed as a part of a combination therapy. As used herein, the phrase “combination therapy” refers to any treatment wherein the methods and compositions disclosed herein are used in combination with another therapy including, but not limited to radiation therapy (radiotherapy), chemotherapy, surgical therapy (e.g., resection), and combinations thereof.

V.A. Radiation Treatment

In some embodiments, the methods and compositions disclosed herein are employed in a combination therapy with radiation treatment. For such treatment of a tumor, the tumor is irradiated concurrent with, or subsequent to, administration of a composition as disclosed herein. In some embodiments, the tumor is irradiated daily for 2 weeks to 7 weeks (for a total of 10 treatments to 35 treatments). Alternatively, tumors can be irradiated with brachytherapy utilizing high dose rate or low dose rate brachytherapy internal emitters.

The duration for administration of a composition as disclosed herein comprises in some embodiments a period of several months coincident with radiotherapy, but in some embodiments can extend to a period of 1 year to 3 years as needed to effect tumor control. A composition as disclosed herein can be administered about one hour before each fraction of radiation. Alternatively, a composition can be administered prior to an initial radiation treatment and then at desired intervals during the course of radiation treatment (e.g., weekly, monthly, or as required). An initial administration of a composition (e.g., a sustained release drug carrier) can comprise administering the composition to a tumor during placement of a brachytherapy after-loading device.

Subtherapeutic or therapeutic doses of radiation can be used for treatment of a radiosensitized tumor as disclosed herein. In some embodiments, a subtherapeutic or minimally therapeutic dose (when administered alone) of ionizing radiation is used. For example, the dose of radiation can comprise in some embodiments at least about 2 Gy ionizing radiation, in some embodiments about 2 Gy to about 6 Gy ionizing radiation, and in some embodiments about 2 Gy to about 3 Gy ionizing radiation. When radiosurgery is used, representative doses of radiation include about 10 Gy to about 20 Gy administered as a single dose during radiosurgery or about 7 Gy administered daily for 3 days (about 21 Gy total). When high dose rate brachytherapy is used, a representative radiation dose comprises about 7 Gy daily for 3 days (about 21 Gy total). For low dose rate brachytherapy, radiation doses typically comprise about 12 Gy administered twice over the course of 1 month. ¹²⁵I seeds can be implanted into a tumor can be used to deliver very high doses of about 110 Gy to about 140 Gy in a single administration.

Radiation can be localized to a tumor using conformal irradiation, brachytherapy, stereotactic irradiation, or intensity modulated radiation therapy (IMRT). The threshold dose for treatment can thereby be exceeded in the target tissue but avoided in surrounding normal tissues. For treatment of a subject having two or more tumors, local irradiation enables differential drug administration and/or radiotherapy at each of the two or more tumors. Alternatively, whole body irradiation can be used, as permitted by the low doses of radiation required following radiosensitization of the tumor.

Radiation can also comprise administration of internal emitters, for example ¹³¹I for treatment of thyroid cancer, NETASTRON™ and QUADRAGEN® pharmaceutical compositions (Cytogen Corp., Princeton, N.J. United States of America) for treatment of bone metastases, ³²P for treatment of ovarian cancer. Other internal emitters include ¹²⁵I, iridium, and cesium. Internal emitters can be encapsulated for administration or can be loaded into a brachytherapy device.

Radiotherapy methods suitable for use in the practice of presently disclosed subject matter can be found in Leibel & Phillips, 1998, among other sources.

V.B. Chemotherapy Treatment

In some embodiments, the methods and compositions disclosed herein are employed in a combination therapy with chemotherapy, also referred to as cytotoxic therapy. Particular chemotherapeutic agents are generally chosen based upon the type of tumor to be treated, and such selection is within the skill of the ordinary oncologist.

Chemotherapeutic agents are generally grouped into several categories including, but not limited to DNA-interactive agents, anti-metabolites, tubulin-interactive agents, hormonal agents, and others such as asparaginase or hydroxyurea. Each of the groups of chemotherapeutic agents can be further divided by type of activity or compound. For a detailed discussion of various chemotherapeutic agents and their methods for administration, see Dorr et al., 1994, herein incorporated by reference in its entirety.

In order to reduce the mass of the tumor and/or stop the growth of the cancer cells, a chemotherapeutic agent should prevent the cells from replicating and/or should interfere with the cell's ability to maintain itself. Exemplary agents that accomplish this are primarily the DNA-interactive agents such as Cisplatin, and tubulin interactive agents. In some embodiments chemotherapeutic agent acts by oxidative stress.

DNA-interactive agents include, for example, alkylating agents (e.g., Cisplatin, Cyclophosphamide, Altretamine); DNA strand-breakage agents (e.g., Bleomycin); intercalating topoisomerase II inhibitors (e.g., Dactinomycin and Doxorubicin); non-intercalating topoisomerase II inhibitors (e.g., Etoposide and Teniposide); and the DNA minor groove binder Plicamycin.

Generally, alkylating agents form covalent chemical adducts with cellular DNA, RNA, and/or protein molecules, and with smaller amino acids, glutathione, and/or similar biomolecules. These alkylating agents typically react with a nucleophilic atom in a cellular constituent, such as an amino, carboxyl, phosphate, or sulfhydryl group in nucleic acids, proteins, amino acids, or glutathione.

Anti-metabolites interfere with the production of nucleic acids by either of two major mechanisms. Some of the drugs inhibit production of deoxyribonucleoside triphosphates that are the immediate precursors for DNA synthesis, thus inhibiting DNA replication. Some of the compounds are sufficiently like purines or pyrimidines to be able to substitute for them in the anabolic nucleotide pathways. These analogs can then be substituted into the DNA and RNA instead of their normal counterparts.

Hydroxyurea appears to act primarily through inhibition of the enzyme ribonucleotide reductase.

Asparagenase is an enzyme which converts asparagine to nonfunctional aspartic acid and thus blocks protein synthesis in the tumor.

Tubulin interactive agents act by binding to specific sites on tubulin, a protein that polymerizes to form cellular microtubules. Microtubules are critical cell structure units. When the interactive agents bind on the protein, the cell can not form microtubules. Tubulin interactive agents include Vincristine and Vinblastine, both alkaloids and Paclitaxel.

Adrenal corticosteroids are derived from natural adrenal cortisol or hydrocortisone. They are used because of their anti-inflammatory benefits as well as the ability of some to inhibit mitotic divisions and to halt DNA synthesis. These compounds include Prednisone, Dexamethasone, Methylprednisolone, and Prednisolone.

The hormonal agents and leutinizing hormones are not usually used to substantially reduce the tumor mass. However, they can be used in conjunction with the chemotherapeutic agents. Hormonal blocking agents are also useful in the treatment of cancers and tumors. They are used in hormonally susceptible tumors and are usually derived from natural sources. These include, but are not limited to estrogens and conjugated estrogens, progestins, and androgens. Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes. Other anti-hormonal agents include anti-estrogenic agents, anti-androgen agents, and anti-adrenal agents such as Mitotane and Aminoglutethimide.

Representative chemotherapeutic agents are presented in Table 2.

TABLE 2
Chemotherapeutic Agents

| Agent Type | Examples |
|---|---|
| Alkylating Agents | |
| Nitrogen mustards | Chlorambucil, Cyclophosphamide, |
| | Isofamide, Mechlorethamine, |
| | Melphalan, Uracil mustard |
| Aziridines | Thiotepa |
| Methanesulfonate esters | Busulfan |
| Nitroso ureas | Carmustine, Lomustine, |
| | Streptozocin |
| Platinum complexes | Cisplatin, Carboplatin |
| Bioreductive alkylators | Mitomycin, Procarbazine |
| DNA strand breaking agents | Bleomycin |
| DNA topoisomerase II inhibitors | Amsacrine, Dactinomycin, |
| | Daunorubicin, Doxorubicin, |
| | Idarubicin, Mitoxantrone, |
| | Etoposide, Teniposide |
| DNA minor groove binder | Plicamycin |
| Anti-metabolites | |
| Folate antagonists | Methotrexate and trimetrexate |
| Pyrimidine antagonists | Fluorouracil, Fluorodeoxyuridine, |
| | CB3717, Azacytidine, |
| | Cytarabine, Floxuridine |
| Purine antagonists | Mercaptopurine, 6-Thioguanine, |
| | Fludarabine, Pentostatin |
| Sugar modified analogs | Cyctrabine, Fludarabine |
| Ribonucleotide reductase | Hydroxyurea |
| inhibitors | |
| Tubulin interactive agents | Vincristine, Vinblastine, Paclitaxel |
| Adrenal corticosteroids | Prednisone, Dexamethasone, |
| | Methylprednisolone, |
| | Prednisolone |
| Hormonal blocking agents | |
| Estrogens and related | Ethinyl Estradiol, |
| | Diethylstilbesterol, |
| | Chlorotrianisene, Idenestrol |
| Progestins | Hydroxyprogesterone caproate, |
| | Medroxyprogesterone, |
| | Megestrol |
| Androgens | Testosterone, Testosterone |
| | propionate; Fluoxymesterone, |
| | Methyltestosterone |
| Leutinizing hormone releasing | Leuprolide acetate; Goserelin |
| hormone agents and/or | acetate |
| gonadotropin-releasing hormone | |
| antagonists | |
| Anti-estrogenic agents | Tamoxifen |
| Anti-androgen agents | Flutamide |
| Anti-adrenal agents | Mitotane, Aminoglutethimide |

A “potentiator” can be any material that improves or increases the efficacy of a pharmaceutical composition and/or acts on the immune system. Exemplary potentiators are triprolidine and its cis-isomer, which can be used in combination with chemotherapeutic agents. Triprolidine is described in U.S. Pat. No. 5,114,951. Other potentiators are procodazole 1H-Benzimidazole-2-propanoic acid; [β-(2-benzimidazole) propionic acid; 2-(2-carboxyethyl)benzimidazole; propazol) Procodazole is a non-specific active immunoprotective agent against viral and bacterial infections and can be used with the compositions disclosed herein. Potentiators can improve the efficacy of the disclosed compositions and can be used in a safe and effective amount.

Antioxidant vitamins such as ascorbic acid, beta-carotene, vitamin A, and vitamin E can also be administered with the compositions disclosed herein.

In some embodiments, methods for inhibiting HIF-1 activity in a normoxic cancer cell are provided. The methods can comprise providing a normoxic cancer cell undergoing cancer therapy that includes chemotherapy and contacting the cancer cell with a composition comprising an effective amount of an inhibitor of HIF-1 activity, whereby HIF-1 activity in the normoxic cancer cell is inhibited. In some embodiments the normoxic cancer cell can expresses inducible nitric oxide synthase (iNOS). As disclosed herein, the inhibitor of HIF-1 activity can be selected from the group consisting of a nitric oxide synthase inhibitor, a nitric oxide scavenger, a STAT-1 inhibitor, and combinations thereof. The nitric oxide synthase inhibitor can comprise an iNOS inhibitor.

In some embodiments a combination therapy of chemotherapy and inhibitor of HIF-1 activity can be employed to prevent cancer therapy-induced activation of HIF-1 in a normoxic cancer tissue. Such a method can comprise providing a subject having a normoxic cancer tissue to be treated with a cancer therapy that includes chemotherapy, administering to the subject a cancer therapy that includes chemotherapy, and administering to the subject an inhibitor of HIF-1 activity, whereby cancer therapy induced activation of HIF-1 in the normoxic cancer tissue is prevented. In some embodiments the normoxic cancer cell can expresses iNOS. As disclosed herein, the inhibitor of HIF-1 activity can be selected from the group consisting of a nitric oxide synthase inhibitor, a nitric oxide scavenger, a STAT-1 inhibitor, and combinations thereof. The nitric oxide synthase inhibitor can comprise an iNOS inhibitor.

In some embodiments the administration of an inhibitor of HIF-1 activity and chemotherapy are performed substantially simultaneously. As used herein, the term “substantially simultaneously” refers to the co-administration of two or more compounds coincident to one another or close in time to one another, e.g. minutes, hours or a day. That is, the administration of an inhibitor of HIF-1 activity can be coincident with the administration of chemotherapy. In some embodiments, the administration of a composition of the presently disclosed subject matter can be before, during, and/or after chemotherapy. In some embodiments, a composition of the presently disclosed subject matter can be administered within a 1, 2, 3, 4, 5, 6, 7, 8, 9, 10, 15, 20 or 30 day period, i.e. before or after, of the administration of chemotherapy. In some embodiments, the duration for administration of a composition as disclosed herein comprises a period of several months coincident with chemotherapy, but in some embodiments can extend to a period of 1 year to 3 years as needed to effect tumor control. Alternatively, a composition can be administered prior to an initial chemotherapy treatment and then at desired intervals during the course of chemotherapy (e.g., weekly, monthly, or as required).

In some embodiments, treatment with a STAT-1 inhibitor, an iNOS inhibitor or NO scavenger should commence prior to or immediately after chemotherapy treatment and should continue with chemotherapy drug administration, which can provide maximum likelihood for full suppression of the iNOS response and subsequent stabilization of HIF-1. The kinetics of the response can vary between individuals, different cancer types, or following use of other drugs, so exact timing of treatment schedules can involve use of biomarkers to monitor the HIF-1 response. As such, in some embodiments the level of HIF-1 activity in a cancer tissue is determined by detecting a plasma level of plasminogen activator inhibitor-1 (PAI-1). In some embodiments the level of HIF-1 activity in a cancer tissue is determined by establishing a ratio between PAI-1 and osteopontin (OPN). In some embodiments the level of HIF-1 activity in a cancer tissue is determined by using positron emission tomography (PET) to detect CAIX, a specific HIF-1 target protein (Apte et al., 2009). In some embodiments the level of HIF-1 activity in a cancer tissue can be determined prior to, during and/or after the administration of chemotherapy.

In some embodiments the presently disclosed subject matter provides a method for implementing a treatment strategy for a cancer in a subject in need of treatment, the method comprising administering to the subject a chemotherapeutic agent, determining a level of HIF-1 activity in a cancer tissue in the subject, and administering an inhibitor of HIF-1 activity to the subject if an elevation of HIF-1 activity is observed after administering the chemotherapeutic agent. The level of HIF-1 activity in the cancer tissue can be determined by detecting a plasma level of plasminogen activator inhibitor-1 (PAI-1). The level of HIF-1 activity in the cancer tissue can be also be determined by establishing a ratio between PAI-1 and osteopontin (OPN).

In some embodiments the presently disclosed subject matter provides a method of treating a cancer in a subject, the method comprising administering a chemotherapeutic agent to the subject, determining whether administering the chemotherapeutic agent to the subject elevates HIF-1 activity in cancer tissue of the subject, and administering a HIF-1 inhibitor to the subject depending on whether elevated HIF-1 activity is observed in the cancer tissue. The level of HIF-1 activity in the cancer tissue can be determined by detecting a plasma level of PAI-1. The level of HIF-1 activity in the cancer tissue can be also be determined by establishing a ratio between PAI-1 and OPN.

In some embodiments the presently disclosed subject matter provides methods of treating a cancer or tumor wherein the cancer or tumor expresses iNOS. As such, in some embodiments the disclosed methods further comprise the treatment of a tumor, cancer cell or cancer tissue expressing iNOS. In some embodiments the disclosed methods comprise determining if the tumor, cancer cell or cancer tissue to be treated or undergoing treatment expresses iNOS. Upon a determination that a tumor, cancer cell or cancer tissue does express iNOS, a HIF-1 inhibitor can be administered to prevent cancer therapy induced activation of HIF-1 and/or inhibit HIF-1 activity to thereby enhance the cancer therapy or sensitize the cancer to cancer therapy. Methods of determining whether a tissue expresses iNOS are known (see, e.g. Example 11 below).

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods Used in the Examples 1-7

Cell Lines and Tissue Culture.

The 4T1 murine mammary adenocarcinoma cell line and B16F10 murine melanoma cells were obtained from the American Type Culture Collection (ATCC, Manassas, Va., United States of America). The two cell lines were cultured in Dulbeccos's modified Eagle's medium (DMEM) supplemented with 10% fetal bovine serum.

Reagents.

CoCl₂, S-nitrosoglutathione (GSNO), potassium 2-(4-carboxyphenyl)-4,4,5,5-tetramethylimidazoline-1-oxyl-3-oxide (carboxy-PTIO), and carrageenan were purchased from Sigma (St. Louis, Mo., United States of America). The proteasome inhibitor MG-132 was purchased from EMD Biosciences, Inc. (San Diego, Calif., United States of America). Luciferin was obtained from Xenogen (Alameda, Calif., United States of America). Nω-nitro-L-arginine methyl ester (L-NAME) and 1400W were purchased from Cayman Chemical (Ann Arbor, Mich., United States of America). N-(6-[biotinamido]hexyl)-3′-(2′-pyridyldithio)-propionamide (biotin-HPDP) was purchased from Pierce Biotechnology (Rockford, Ill., United States of America).

Plasmids and Cloning Procedures.

The HIF-1α bioluminescence reporter (ODD-luc) construct was created by fusing PCR product of ODD domain of HIF-1α (GENBANK® Accession No. U59496) to the 5′ end of firefly luciferase reporter gene. Along with this construct, a luciferase expression vector in which luciferase gene was driven by the CMV promoter was used as a control. A C533S ODD mutation was achieved by in vitro site-directed mutagenesis. ODD fragments with or without the mutation were cloned into pCMV-3Tag-4B Epitope Tagging Mammalian Expression vector (Stratagene, La Jolla, Calif., United States of America). The full length mouse VHL gene (GENBANK® Accession No. S76748) was cloned form mouse tissue and tagged with HA tag by PCR. All constructs were sequence-verified.

siRNA.

siRNA sequences targeted to the VHL gene were designed by using an Internet-based program available at the website of Ambion Inc. (Austin, Tex., United States of America). A retroviral siRNA expression vector (pSilencer 5.1-U6 Retro from Ambion Inc.) was used to stably introduce the following siRNA sequence targeted to VHL gene to 4T1 cells: AACATCACATTGCCAGTGTAT (SEQ ID NO: 17). pSilencer 5.1-U6 Scrambled siRNA (Ambion Inc.) was used as a negative control.

Imaging Luciferase Activity.

Luciferase expression/activity was detected and quantified as relative light units (RLUs) by using the Xenogen IVIS™ imaging system and associated LIVING IMAGE® software (Xenogen, Alameda, Calif., United States of America). For in vitro observations, cells transfected with luciferase reporter gene constructs were grown in 12-well or 24-well cell culture plates. After cells reached 80% confluence, they were treated with the different chemicals or were cultured in the hypoxic chamber (Sheldon Manufacturing, Inc., Cornelius, Oreg., United States of America). At the times indicated, luciferin (150 μg/ml) was added and the plates were imaged for luciferase expression. For in vivo experiments, treated tumor-bearing mice received an i.p. injection of luciferin (150 mg/kg) during isofluorane anesthesia. Repeated images of luciferase expression/activity were acquired following manufacturer's specified procedures.

Animal Experiments.

Female NIH Swiss nude mice, C57BL/6 mice were purchased from the National Cancer Institute (NCI; Fredrick, Md., United States of America). Mice with a genetic disruption of the inducible nitric acid synthase gene (C57BL/6 iNOS^{-/-}) were obtained from the Jackson Laboratory (Bar Harbor, Me., United States of America). Female BALB/c mice were obtained from Charles River Laboratories (Raleigh, N.C., United States of America). Animals were maintained and cared for in accordance with the Duke University Institutional Animal Care and Use Committee guidelines. For tumor implantation, 4T1 tumors were grown in nude mice or in syngeneic BALB/c mice, and B16F10 melanoma in syngeneic C57BL/6 or iNOS^{-/-} mice. About 5×10⁵ wild type- or luciferase reporter gene-transfected tumor cells were injected subcutaneously (s.c.) into mice in 50 μl of PBS solution in the hind legs of ketamine/xylazine-anesthetized mice. When the tumors reached 6-8 mm in diameter, mice were randomly assigned to experimental groups. To image the luciferase activity, single dose of 6 Gy X-ray was given to the tumor on the right leg and this day was set as day 0. The luciferase activity was imaged everyday or every other day for ten days. For the tumor growth delay assay, radiotherapy was employed in its clinical mode: tumors were treated with 3 fractions of 6 Gy every other day from day 0. Tumor growth was then followed by use of a caliper every 2 days. Tumor volume was calculated using the following formula: volume=(length×width²)/2. Where indicated, mice received the Pan-NOS inhibitor L-NAME (500 mg/L) or the iNOS-selective inhibitor 1400W (50 mg/L) in the drinking water one day before X-ray treatment. Drinking water was renewed daily until animal sacrifice.

ELISA and Western Blot Analysis.

Tumor homogenates and tissue culture samples were both used for protein analysis. All results were normalized for total sample protein contents, determined by using a Bradford-based assay (BIO-RAD, Hercules, Calif., United States of America). Nuclear extracts were prepared by use of the NUCBUSTER™ Protein Extraction Kit (Novagen, San Diego, Calif., United States of America). HIF-1 binding activities in tumor homogenates were quantified with the TRANSAM™ HIF-1 ELISA Kit (Active Motif, Carlsbad, Calif., United States of America) with the use of an antibody against mouse HIF-1α (Novus Biologicals, Littleton, Colo., United States of America). VEGF levels were assayed using the mouse VEGF quantikine ELISA Kit (R&D systems, Minneapolis, Minn., United States of America). HIF-1α levels in tissue culture samples were determined by Western Blot using a polyclonal rabbit anti-HIF-1 antibody (Novus Biologicals) detected with horseradish peroxidase (HRP)-conjugated donkey anti-rabbit secondary antibody (Santa Cruz Biotechnology, Santa Cruz, Calif., United States of America).

Macrophage Depletion Experiments.

Macrophage depletion was achieved in mice as described in Muller et al., 2005. Briefly, nude mice received repeated i.p. injections of 2 mg carrageenan at 6, 3, and 1 day before s.c. injection of 4T1 tumor cells, after which mice were injected once per week until the end of the experiment.

Immunohistochemical Stainings.

Immunofluorescence stainings were performed on tumors biopsied 5 days after irradiation (6 Gy). Cryoslices were fixed in 4% paraformaldehyde. Endogenous peroxidases were quenched with 3% H₂O₂. slices were then blocked with 10% normal serum, probed with a primary antibody, and revealed with a secondary antibody coupled to FITC (to reveal CD68 and HIF-1α) or to TRITC (for iNOS detection; Jackson ImmunoResearch Labs, Inc., West Grove, Pa., United States of America). Primary antibodies were: polyclonal rat anti-CD68 to label macrophages (BD PHARMINGEN™, San Jose, Calif., United States of America), polyclonal goat anti-iNOS (Santa Cruz Biotechnology), and polyclonal rabbit anti-HIF-1α (Novus Biologicals). Cryoslices were examined with a Zeiss Axioskop microscope equipped for fluorescence. Digitized pictures were overlaid by using the METAMORPH® software from Molecular Devices Corp. (Sunnyvale, Calif., United States of America).

Vessel staining was performed on tumor cryoslices by using the VECTASTAIN® ABC and the NOVARED™ kits from Vector Laboratories (Burlingame, Calif., United States of America), according to the manufacturer's protocol. The primary anti-CD31 antibody (Santa Cruz Biotechnology, Santa Cruz, Calif.) was labeled with a secondary biotinylated anti-rabbit antibody (Jackson ImmunoResearch Labs, Inc.). Slices were counterstained using Harris' hematoxilin. Vascular density was determined by counting CD31-positive structures in 5 random fields per tumor.

In Vitro Interaction Assay for ODD and VHL Protein.

4T1 cells grown in 35 mm dishes were transfected with 3 μg of pCMV-ODD-3Myc, including c-myc-tagged wild type ODD and its mutant version, or 3 μg of pCMV-HAVHL encoding HA-tagged full-length VHL by using lipofectamine 2000 (Invitrogen, Carlsbad, Calif., United States of America). 24 hours later, cells were subjected to 1 mM GSNO for 8 hours. Cells were then scraped off the dishes and collected. To each cell pellet 300 μl lysis buffer (50 mM Tris, 150 mM NaCl, 0.5 μM ferrous chloride, 0.5% NP-40, 0.5 μM MG-132, protease inhibitor cocktail, pH 7.5) was added. After centrifugation (15,000×g for 30 min), supernatants were transferred to fresh tubes and the input ODD and VHL were detected by Western Blot analysis using antibodies against C-myc tag or HA tag (Novus Biologicals). 0.5 mg of supernatant from ODD-c-myc or C533S-ODD-cmyc-expressing cells were mixed with 0.25 mg of the supernatant from HA-VHL expressing cells and incubated at 4° C. for 2 hours. The co-immunoprecipitation was achieved by the addition of 20 μl of anti-HA antibody (agarose immobilized; Novus Biologicals). Beads were collected, washed three times with 1 ml washing buffer(20 mM Tris, 100 mM NaCl, 1 mM EDTA, 0.5% NP-40), supplemented with 50 μl 2×SDS-PAGE sample buffer, and boiled at 95° C. for 10 minutes. Beads were removed by centrifugation, and supernatants were loaded on 12% SDS-gels. The amount of ODD-c-myc or C533S-ODD-c-myc that had been pulled down with HA-VHL was probed with a primary anti-c-myc antibody (Novus Biologicals), and revealed with an anti-goat secondary antibody (Santa Cruz Biotechnology). After membrane stripping (RESTORE™ Western Blot Stripping Buffer, Pierce Biotechnology), immunoprecipitated HA-VHL was labeled with a primary antibody against HA (Novus Biologicals), and revealed with a secondary anti-goat antibody (Santa Cruz Biotechnology).

Biotin Switch Assay.

Biotin switch assay was performed as described (Jaffrey & Snyder, 2001). Briefly, 4T1 cells were transfected with pCMV-ODD-3Myc. 24 hours later, cells were treated with 1 mM GSNO for 8 h. Cells were then homogenized by 26G needle in HEN buffer (250 mM HEPES-NaOH pH 7.7, 1 mM EDTA, 0.1 mM Neocuproine), and then centrifuged at 1000×g for 10 minutes at 4° C. Supernatant (300 μg) was added to 4 volumes of blocking buffer (9 volumes of HEN buffer plus 1 vol 25% SDS, 20 mM MMTS) at 50° C. for 20 minutes with frequent vortexing. The MMTS was then removed by desalting three times with the BIO-SPIN® 6 column (Bio-Rad, Hercules, Calif., United States of America) pre-equilibrated in HEN buffer. To the eluate was added biotin-HPDP (final concentration of 2 mM) prepared fresh as a 4 mM stock in DMSO from a 50 mM stock suspension in DMF. Sodium ascorbate was added to a final concentration of 1 mM. After incubation for 1 hour at 25° C., biotinylated proteins were precipitated by streptavidin-agarose beads (Pierce Biotechnology, Rockford, Ill., United States of America). The streptavidin-agarose beads were then pelleted and washed 5 times with HENS buffer. The biotinylated proteins were eluted by SDS-PAGE sample buffer and subjected to Western blot analysis. The biotinylated ODD was detected by use of an antibody against the c-myc tag.

Statistics.

Student's t test, one-way and two-way ANOVA were used where indicated. In growth delay experiments, the numbers of days for tumors to reach 5× their initial volume were used for comparing different treatment groups. P<0.05 was considered to be statistically significant.

### Example 1

### A Novel Reporter for Non-Invasive, In Vivo Observation of HIF-1 Activity

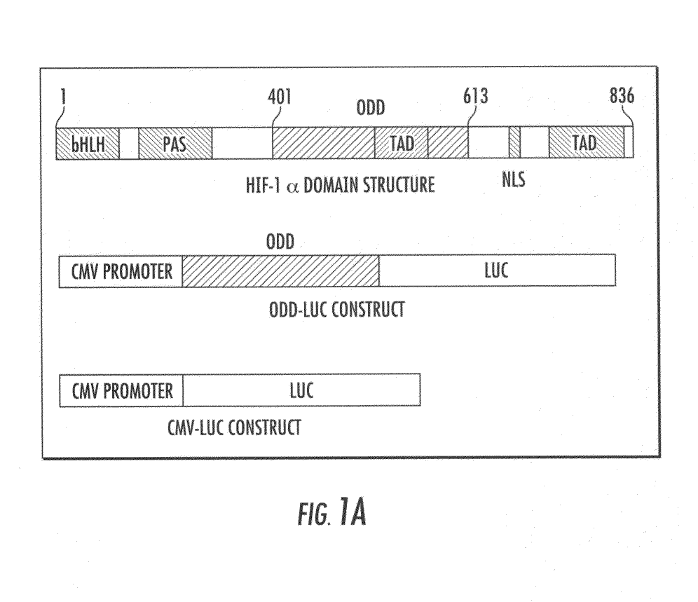

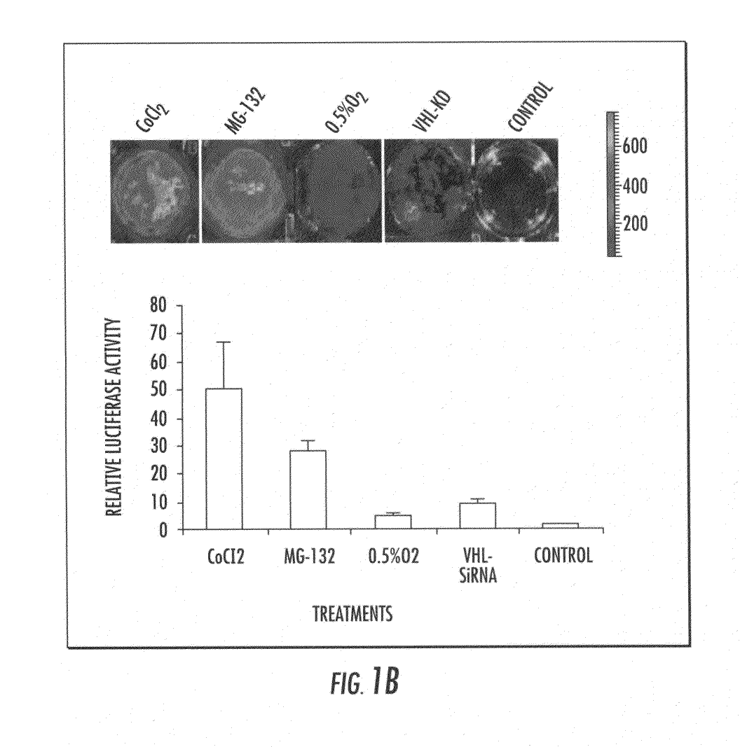

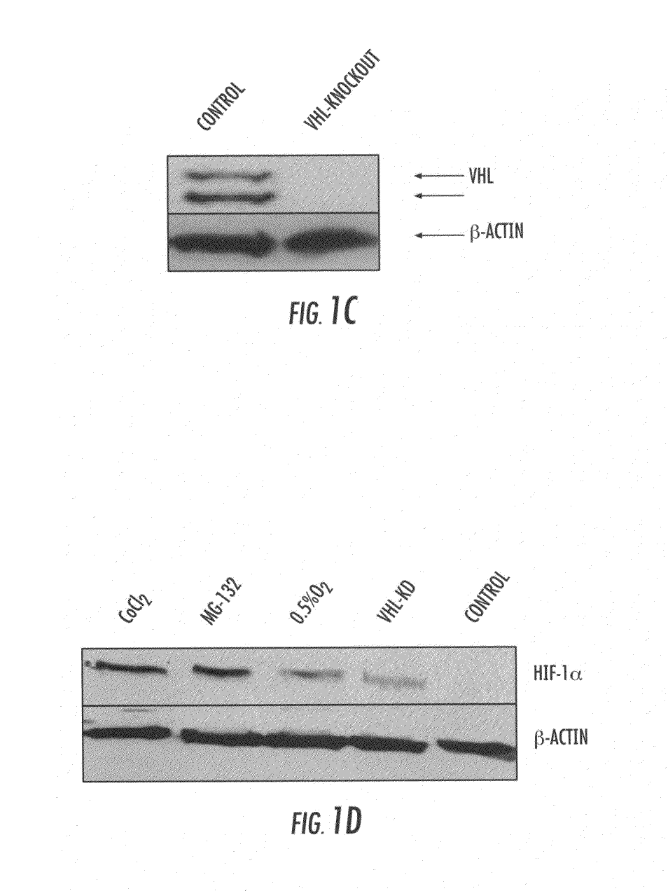

HIF-1 activity is difficult to study in vivo because of the very short half-life of the HIF-1α subunit (Yu et al., 1998). A strategy was adopted in which the oxygen-dependent degradation (ODD) domain of the protein was fused with the firefly luciferase gene (luc; see FIG. 1A). This approach took advantage of the fact that the stability of the HIF-1α subunit (and hence the activity of HIF-1) is mainly regulated by the ODD. It has been shown that the modification of key proline residues by proline hydroxylases (PHDs) under normoxic conditions render the HIF-1α susceptible to binding by VHL and subsequent degradation by the proteasome system.

Thus, it was reasoned that the fusion protein would recapitulate the regulation of HIF-1α and serve as noninvasive reporter of HIF-1α activity. When introduced into several tumor cell lines and evaluated under various treatment conditions, the reporter fulfilled expectations. While background luminescence arising from reporter gene expression was very low, it rose significantly after cellular exposure to hypoxia, CoCl₂ (an established inhibitor of HIF-1α degradation), or MG132 (a proteasome inhibitor), closely mimicking the known regulation of HIF-1α (see FIG. 1B).

The successful recapitulation of HIF-1α stability regulation was further confirmed by transfecting the reporter cells with a VHL-targeted siRNA. The siRNA effectively reduced the level of the VHL expression (see FIG. 1C), which led to significant increases in ODD-luc level, consistent with the role of VHL as the main mediator of HIF-1α ubiquitylation and degradation. Western blot analysis showed a parallel increase in endogenous HIF-1α level after various treatment conditions (FIG. 1D), further validating the ODD-luc reporter as a surrogate marker for HIF-1α.

### Discussion of Example 1

The ODD-luc reporter had a much better dynamic range for the detection of HIF-1α levels than previous promoter-based approaches. In previous studies dealing with the radiation-induced HIF-1α activation (Moeller et al., 2004; Moeller et al., 2005), the in vivo data were mostly obtained with a GFP reporter containing an artificial hypoxia-responsive promoter (HRP). As GFP is a very stable protein (with half life exceeding 24 hours) and the artificial HRP promoter is subject to HIF-independent biological influences that lead to high background activity, the sensitivity of the HRP-GFP reporter was very limited, with a dynamic range limited to 1-3 fold over background level. In addition, HIF-GFP-based experiments had to be carried out with invasive tumor models such as the dorsal skinfold window chamber tumors (Huang et al., 1999) to obtain quantitative data or to conduct repeated measurements. With the new reporter, any murine tumor system can be monitored with the dynamic range of the reporter increased from 1 to 100 fold over background, and repeated measurements acquired non-invasively by means of in vivo optical imaging such as the Xenogen IVIS™ imaging system (Contag et al., 1998; Zhang et al., 2004a).

### Example 2

### Radiation-Induced HIF-1α Stabilization in Tumors

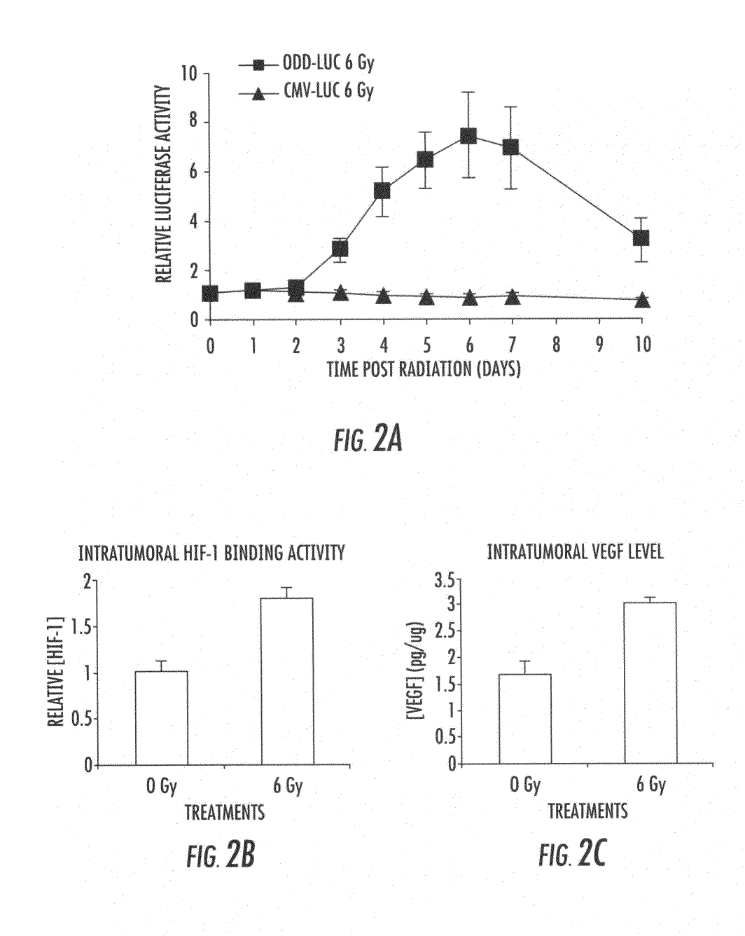

In order to observe HIF-1α regulation after treatment response, 4T1 murine breast tumor cells stably transduced with the ODD-luc reporter gene were implanted subcutaneously into mice. After the tumors reach 6-8 mm in diameter, they were irradiated and followed for ODD-luc expression using the Xenogen IVIS™ system. From day 3 after irradiation, the level of HIF-1α, as determined by ODD-luc, appeared to increase linearly over 3 days. It peaked at around 6 days and fall back to background levels after day 10 (see FIG. 2A). The differences between the irradiated and sham-irradiated groups were highly significant from day 3 (p<0.01).

Radiation-induced stabilization of ODD-luc was accompanied by increases in HIF-1 promoter binding activities to the corresponding HRE binding element (see FIG. 2B) and upregulation of a downstream target gene, vascular endothelial growth factor (VEGF; see FIG. 2C). Similar results were obtained with two other tumor models, B16.F10 melanoma model and the CT26 colon cancer model. These results indicated that radiation induced a persistently increasing level of HIF-1α expression and activity. While radiation has been shown to activate HIF-1α in previous studies (Moeller et al., 2004), the pattern of in vivo induction such as the one disclosed herein had never been observed previously.

### Example 3

### Role of Nitric Oxide in Mediating Radiation-Induced HIF-1α Activation

The cause of radiation-induced HIF-1α stabilization is not understood. One possibility is that radiation creates a more hypoxic condition in the tumor microenvironment than pre-treatment, which causes the stabilization of HIF-1α through the prolyl hydroxylase (PHD) pathway. However, this is highly unlikely. Previous studies have indicated no significant changes (Brizel et al., 1999; Brizel et al., 1996) in the level of hypoxia in tumor following radiation. Indeed, it had been shown that tumor oxygen tension actually increases after irradiation due to a reduced cell proliferation and tumor cell death. Measurements of 4T1 tumors after irradiation indicated a similar scenario (Moeller et al., 2004).

The co-inventors' previous studies had indicated that radiation induced free radicals are at least partially involved in the activation of so-called “stress granules” (Moeller et al., 2004). However, the identity of the free radicals involved in that response is not clear.

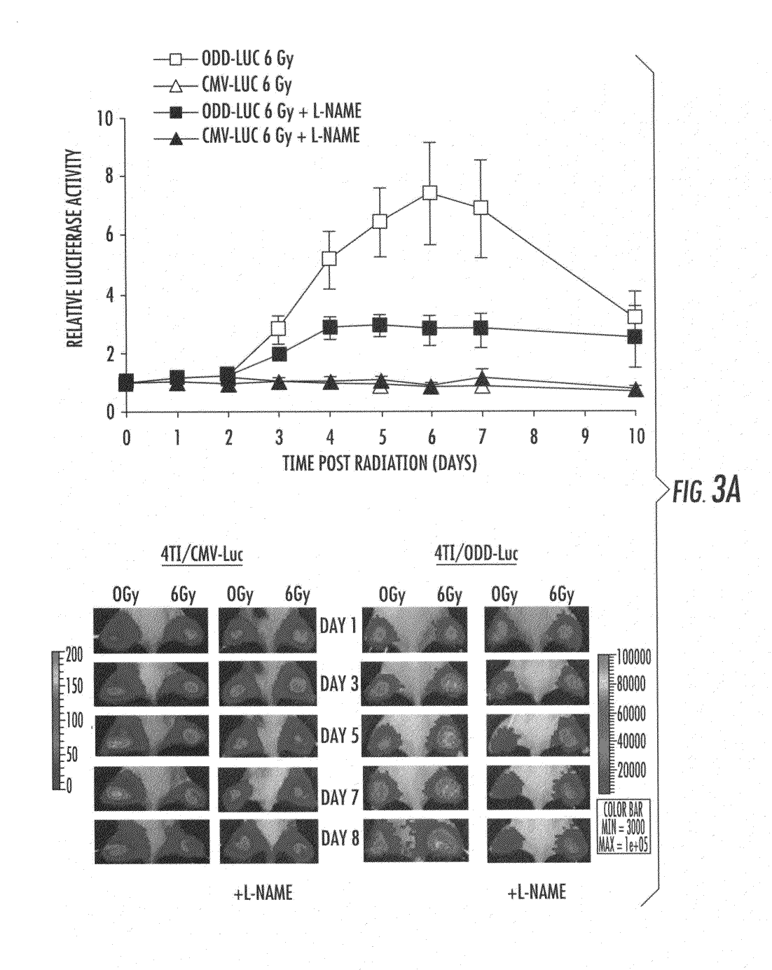

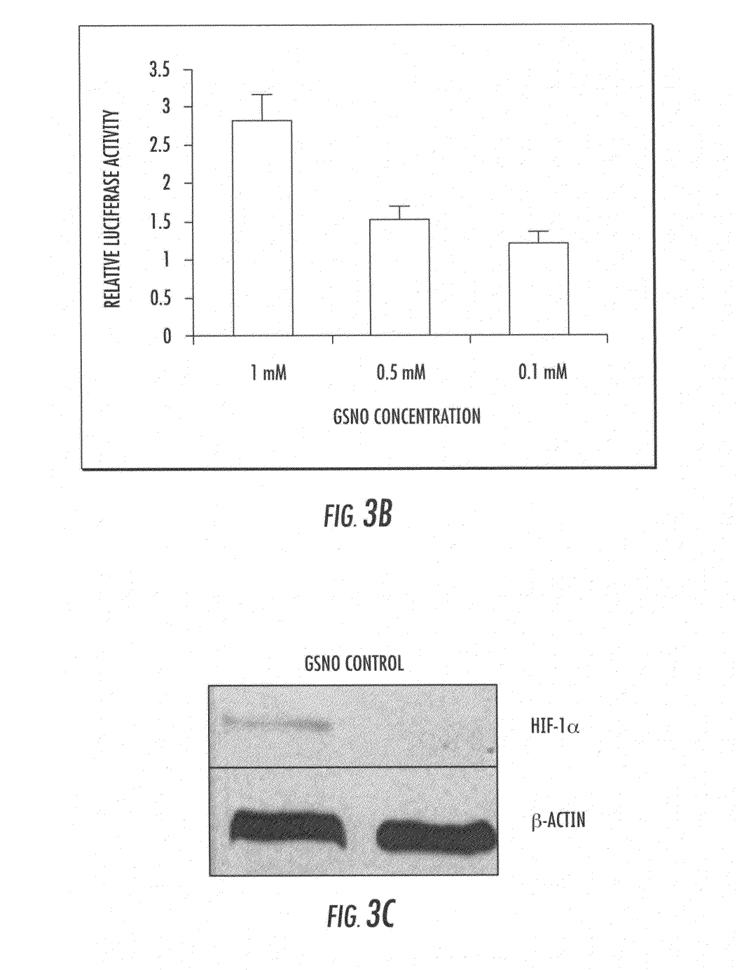

After evaluation with various agents, nitric oxide (NO) was determined to be the main free radical species that was responsible for radiation-induced HIF-1α activation (see FIG. 3A). The administration of L-NAME, a potent non-specific inhibitor of nitric oxide synthases (NOS), to mice effectively attenuated radiation-induced HIF-1α stabilization in tumors, as shown by the loss of ODD-luc signal. As NOS are the major source of NO in vivo, the presently disclosed results indicated that NO played a pivotal role in radiation-induced HIF-1α stabilization. Control experiments indicated that NO produced by NOS did not influence the activity of constitutively expressed luciferase activity, confirming the role of NO in regulating ODD (and hence HIF-1α) stability (see FIG. 3A).

The role of NO was further confirmed in cell culture assays. Treatment of 4T1-ODD-luc cells with the NO donor S-nitrosoglutathione (GSNO) effectively induced dose-dependent HIF-1α activation, similar to treatment with ionizing radiation (see FIG. 3B). Western blot analysis of the GSNO treated cells clearly indicated endogenous HIF-1α induction (FIG. 3C. A NO scavenger, carboxy-PTIO(4-carboxyphenyl-4,4,5,5-tetramethylimidazoline-3-oxide-1-oxyl), effectively suppressed HIF-1α activation by GSNO (FIG. 3D), demonstrating that NO is directly responsible for the observed ODD-luc accumulation.

### Example 4

### Inducible Nitric Oxide Synthase as a Major Source of NO in Radiation-Induced HIF-1α Activation

The source of the NO that stimulates HIF in irradiated tumors in vivo was investigated. Of the three NOS isoforms, inducible NO synthase (iNOS), is the most likely candidate because, unlike neuronal and endothelial NOS, which are constitutively activated in healthy tissues, it is exclusively expressed and activated in pathological tissues such as tumors, where it can produce high micromolar levels of NO. Moreover, tumors usually contains significant number of macrophages (Colombo & Mantovani, 2005; Lewis & Murdoch, 2005), which express/activate their iNOS as part of their immunoeffector activity and thus provide a ready source of NO upon activation.

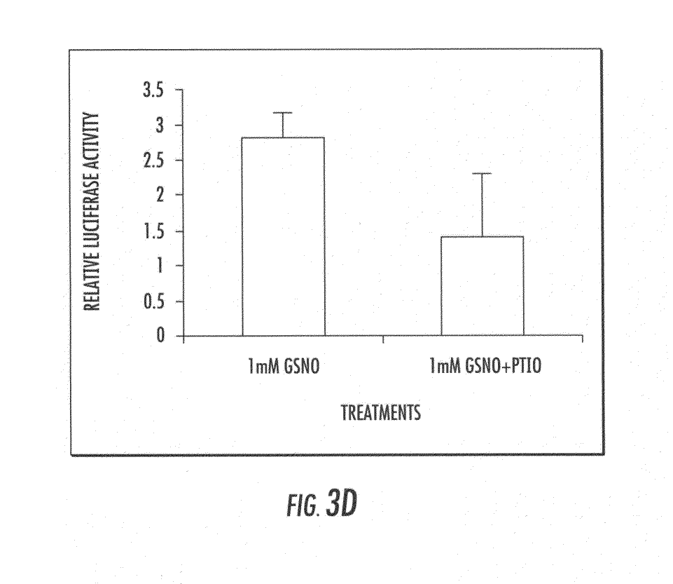

To pinpoint the source of NO, two series of experiments were performed. In the first series of experiments, the iNOS-specific inhibitor, 1400W (Alderton et al., 2001; Thomsen et al., 1997), was used to examine radiation-induced HIF-1α induction in ODD-luc-transduced 4T1 tumors. The results indicated that 1400W attenuated radiation-induced ODD-luc in 4T1 as potently as the general NOS inhibitor L-NAME (see FIG. 4A). This observation indicated that iNOS is the main mediator of radiation-induced HIF-1α stabilization.

In the second series of experiments, C57BL/6 mice with targeted disruption of the iNOS gene (iNOS^{-/-}) were implanted with syngeneic B16F10 melanoma cells stably transduced with ODD-luc gene. The tumors were then irradiated and observed for HIF-1α activation. A significant attenuation of radiation-induced ODD-luc induction in the tumors grown in iNOS^{-/-} mice compared to wild-type controls was observed. In fact, ODD-luc suppression in iNOS^{-/-} animals and in wild type mice treated with L-NAME were of similar amplitude (see FIG. 4B), indicating that L-NAME-suppressed HIF-1α activation in the wild type mice was attributable to the inhibition of iNOS.

### Example 5

### Macrophages are a Major Source of iNOS and NO in Radiation-Induced HIF-1α Activation

Previous studies have indicated that macrophages are a rich source of NO and that the tumor microenvironment is abundantly populated with macrophages. In light of this information and together with the results presented hereinabove, it was hypothesized that tumor-associated macrophages might play a significant role in radiation-induced HIF-1α induction. This would also be consistent with previous findings that tumor-associated macrophages play important roles in regulating tumor angiogenesis, at least partially through NO release (Leek et al., 2000; Leek et al., 2002; Varney et al., 2002).

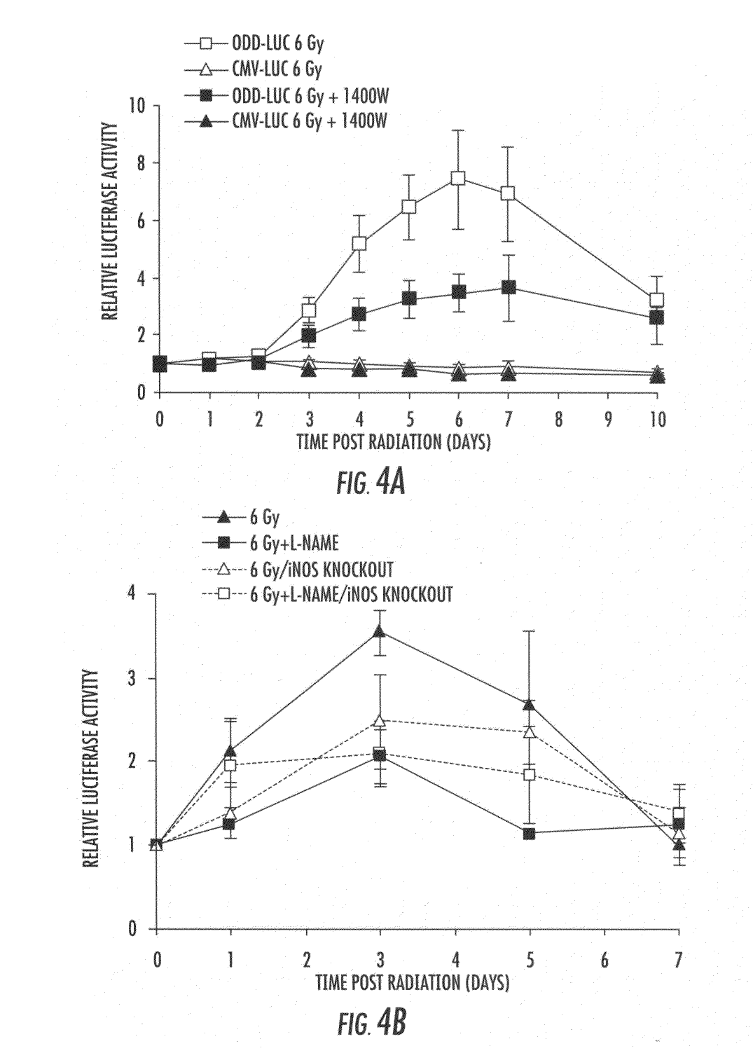

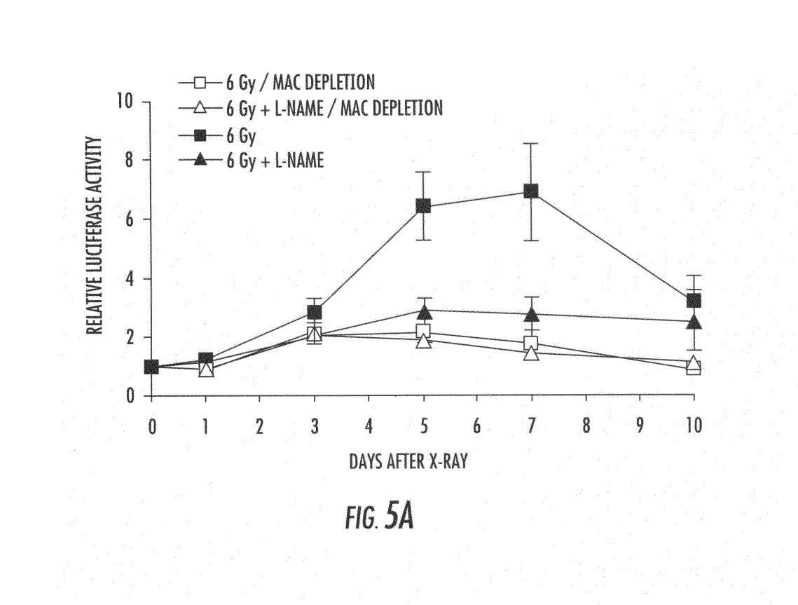

In order to investigate the potential involvement of macrophages in HIF-1α activation, radiation-induced ODD-luc activation in mice that had been chemically depleted of macrophages through the use of carrageenan was measured (Goldmann et al., 2004; Muller et al., 2005; Udono et al., 1994). The results were very similar to those obtained in iNOS^{-/-} mice (see FIG. 4). A significant reduction in radiation-induced ODD-luc activation and the loss of L-NAME inhibition of the activation in tumors in mice with macrophage depletion was observed (FIG. 5A).

These results clearly established that iNOS in tumor-associated macrophages was the main source for the NO that was involved in radiation-induced HIF-1α activation. See FIG. 20. Immunohistochemistry analysis further confirmed that irradiation of the tumor increased the number of tumor-associated macrophages and activated the iNOS gene in these macrophages (see FIG. 5B, left panel). The results presented herein also confirmed that activated iNOS gene expression was accompanied by concomitant HIF-1α activation in tumors (FIG. 5B, right panel).

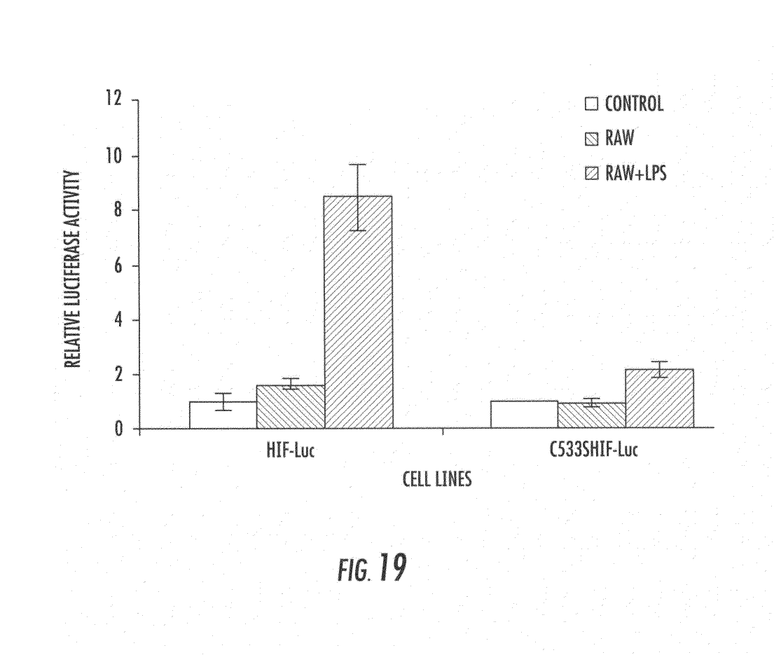

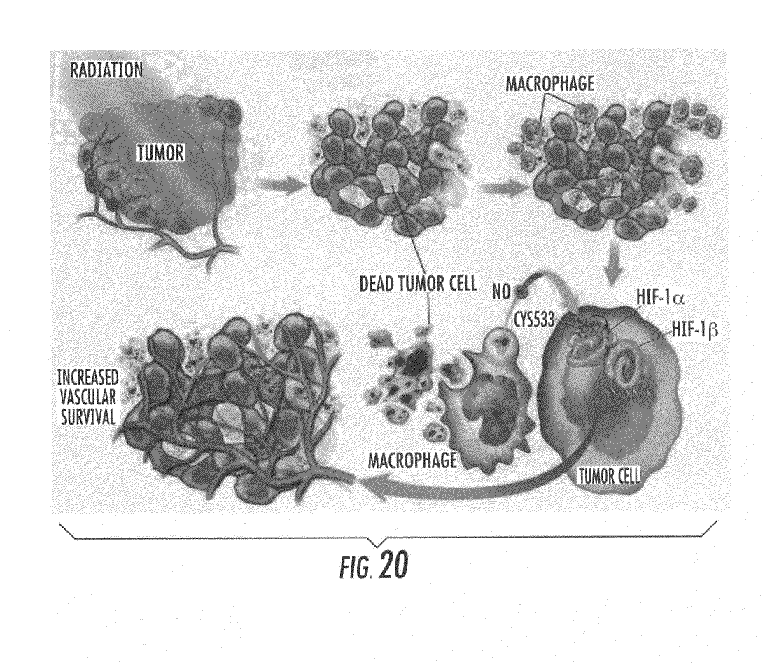

Furthermore, FIG. 19 is a bar graph presenting the results of C533S mutation on activated macrophage-mediated HIF-1α stabilization by use of a full-length HIF-1α-luciferase fusion reporter gene. 4T1 cells stably transduced with either the wild-type full-length HIF-Luc reporter gene or its mutant version C533S HIF-luc gene were cultured in 24-well plates to 50% confluence (1.0×10⁵ cell per well). About 1.5×10⁵ RAW 264.7 (mouse macrophage) were added after 24 hours. Two hours later, 10 μg/ml of LPS and 0.1 ng/ml of IFN-γ were added to the medium. Twelve hours later, cells were imaged for luciferase activity in the Xenogen IVIS200 system. The addition of LPS, which activated the wild type HIF-1α-luc fusion reporter significantly (p<0.005, t test), had minimal effect on the C533S mutant HIF-1α fusion reporter.

### Example 6

### The Molecular Mechanism of HIF-1α Activation by NO

The aforementioned experiments provided strong evidence that NO generated by tumor-associated macrophages played roles in radiation-induced HIF-1α activation. However, the exact molecular mechanism of how NO induces stabilization of HIF-1α remained unclear.

In theory, there are at least two ways NO can influence HIF-1α: the inactivation of upstream prolyl hydroxylases and/or the direct modification of the ODD domain. Others have shown that NO can inhibit the activity of the prolyl hydroxylases (PHDs), which can result in the stabilization of HIF-1α (Metzen et al., 2003). However, inhibition of PHDs did not appear to account for all the NO-induced HIF-1α activation. It was therefore reasoned that a direct modification of the ODD domain could also participate in HIF-1 activation.

A previous report suggested that, although all 13 cysteine residues in the purified HIF-1α protein are susceptible to nitrosylation in test tubes, only 3-4 can be nitrosylated in cells in cultured cells (Sumbayev et al., 2003). However, the biological significance of these nitrosylations on HIF-1α stability has not been identified.

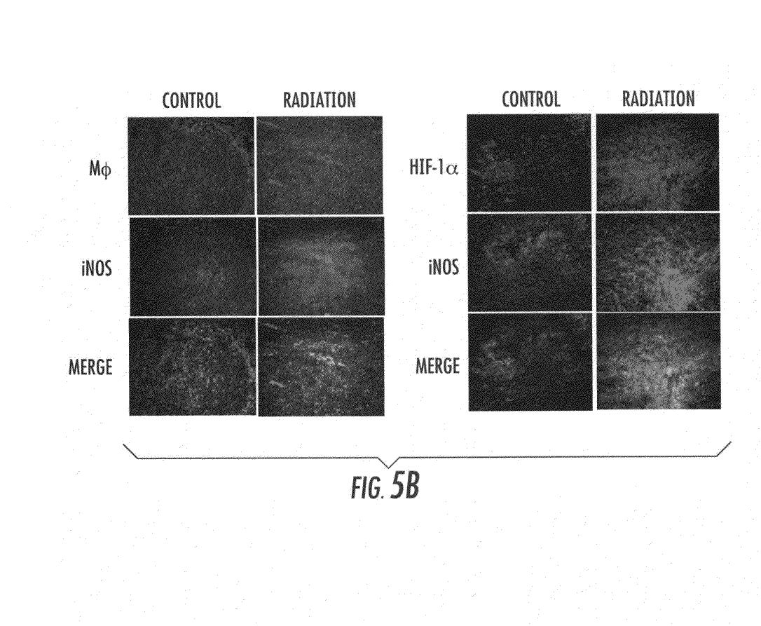

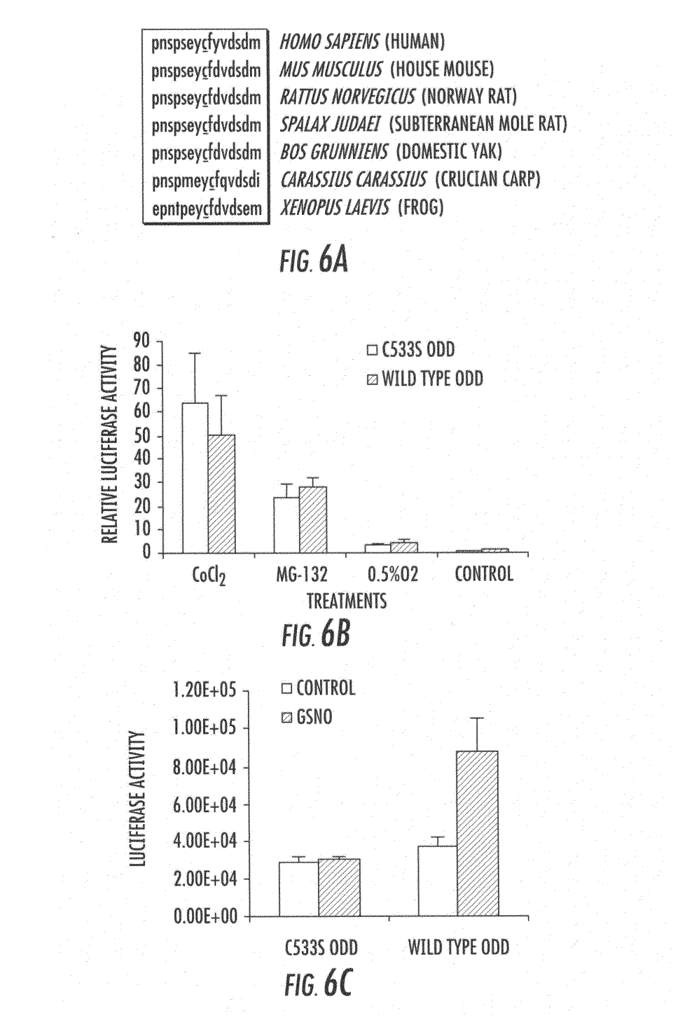

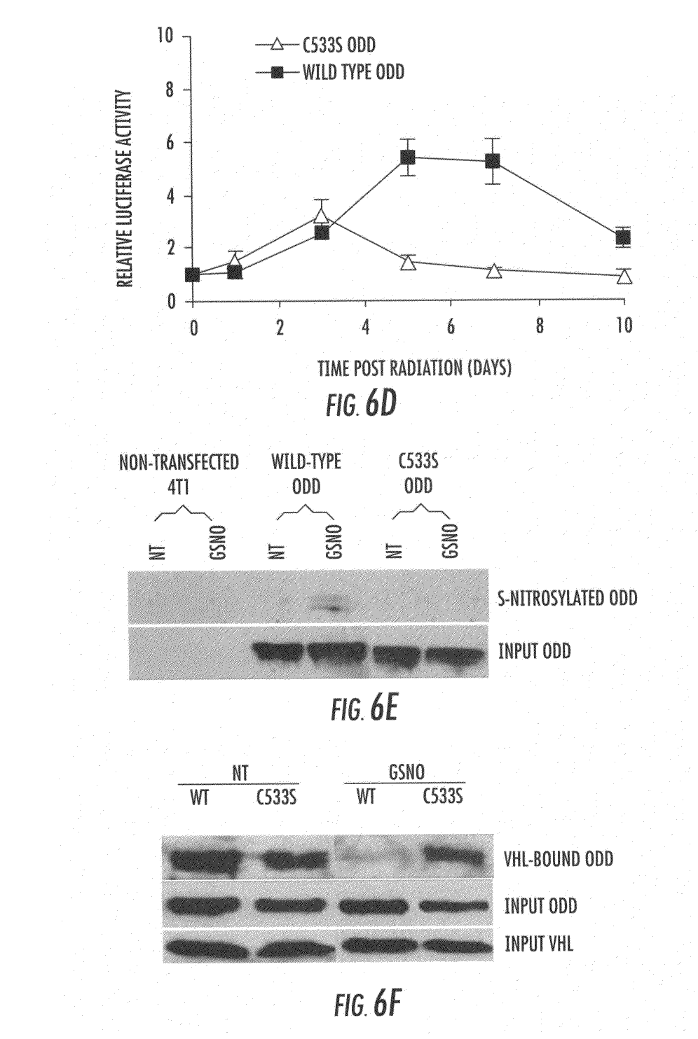

Thus, NO might stabilize the HIF-1α through S-nitrosylation of ODD domain during radiotherapy. To test this hypothesis, a mutant (C533S) involving the only Cys residue in the murine HIF-1α ODD domain, Cys533, was generated. This residue corresponds to Cys520 (which also is the only Cys the human ODD domain) in human HIF-1α and is conserved among a wide spectrum of vertebrate species that included human, mouse, rat, frog, etc. (see FIG. 6A). The replacement of the cysteine by a serine was chosen because the only difference between these amino acids is that the thiol (—SH) group of cysteine is replaced by the hydroxyl (—OH) group of serine, thereby preventing S-nitrosylation. The likelihood that the point mutation will alter the 3-D structure of ODD is thus minimal. The C533S ODD domain was then fused with the luciferase reporter gene, transfected into 4T1 tumor cells, and examined for its activation in comparison with wild type ODD-luc in vitro and in vivo.

In vitro, the background expression level of mutant C533S-ODD-luc was very low, similar to wild type (see FIG. 6B). However, when C533S-ODD-luc was subjected to hypoxia, proteasome inhibition, or CoCl₂ exposure, significant inductions, similar to wild-type, were observed (see FIG. 6B), indicating that the mutation did not cause any gross structural perturbation that would disrupt normal processing by upstream PHDs and downstream VHL and proteasome.

However, when the mutant C533S-ODD-luc transduced cells were exposed to the NO donor GSNO, induced ODD-luc expression was almost absent, in sharp contrast to wild type ODD-luc transduced cells, which had significant GSNO induction (see FIG. 6C).

In vivo, background levels of mutant C533S-ODD-luc transduced tumors were similar to what was observed in wild type ODD-luc transduced tumors (see FIG. 6D). However, the C533S mutation significantly attenuated radiation-induced ODD-luc activation in vivo (see FIG. 6D, days 5, 7, and 10), indicating that S-nitrosylation of the Cys533 residue in the HIF-1α protein played a critical role in regulating the stabilization of HIF-1α after radiation therapy.

The direct proof for S-nitrosylation of HIF-1α at C533 came from “biotin switch” experiments (Jaffrey & Snyder, 2001) in which direct chemical evidence for the nitrosylation of the ODD domain was sought. In the absence of GSNO treatment, neither wild type ODD nor C533S-ODD was S-nitrosylated (see FIG. 6E). S-nitrosylation was clearly observed in wild type ODD upon GSNO treatment, but completely absent in C533S-ODD after GSNO treatment (see FIG. 6E), demonstrating that C533 was S-nitrosylated in the cellular environment with a sufficient amount of NO.

The site-directed mutagenesis experiments disclosed herein further suggested that nitrosylation at Cys533 rendered the HIF-1α protein resistant to degradation by preventing the binding of HIF-1α by VHL. To examine this possibility, the effects of NO and of C533S on the binding of ODD with VHL in ODD-transfected tumor cells were tested. Co-immunoprecipitation results revealed that the strong binding of wild-type ODD with VHL in the absence of NO was completely abolished in cells exposed to GSNO (see FIG. 6F). Strikingly, this regulation was completely lost in cells expressing C533S-ODD, which is consistent with the continuous degradation of the mutated ODD in the presence of NO (FIGS. 6C and 6D). Taken together, the in vivo and in vitro results (see FIG. 6A-F) provided compelling evidence that NO-mediated stabilization of HIF-1α is largely mediated by S-nitrosylation of the Cys533 in the ODD domain. See schematic illustration in FIG. 20.

### Example 7

### The Functional Importance of NO-Mediated HIF-1α Activation During Cancer Therapy

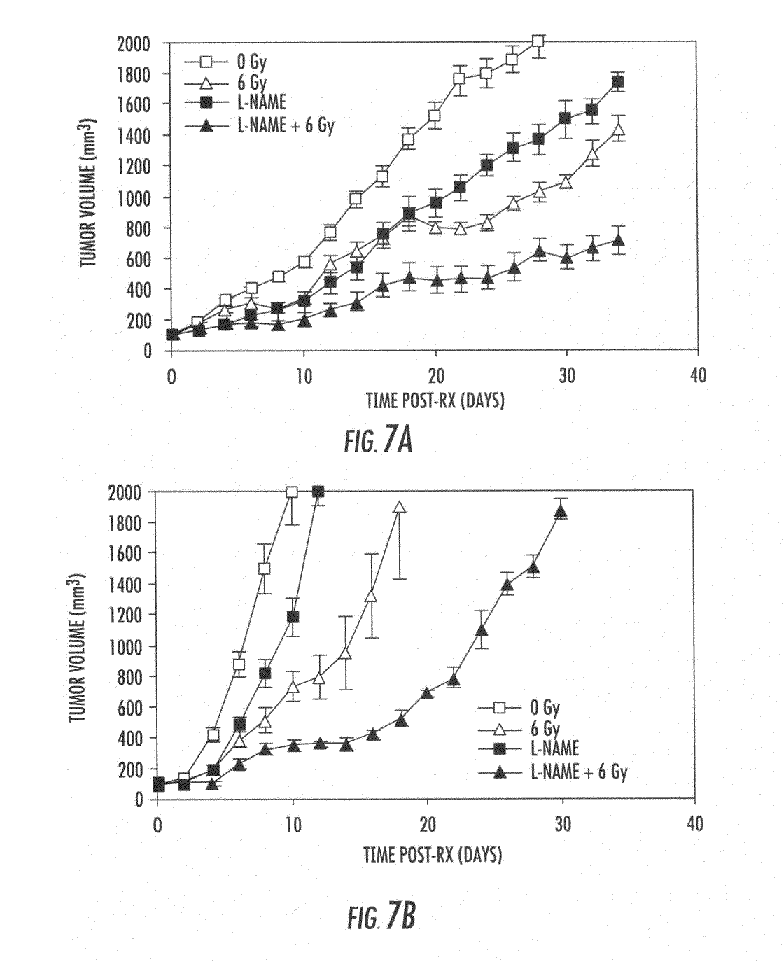

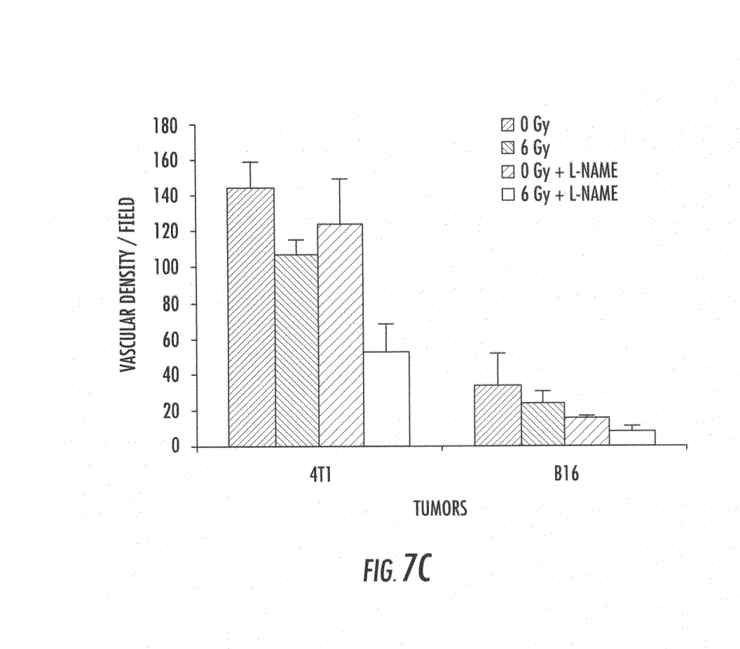

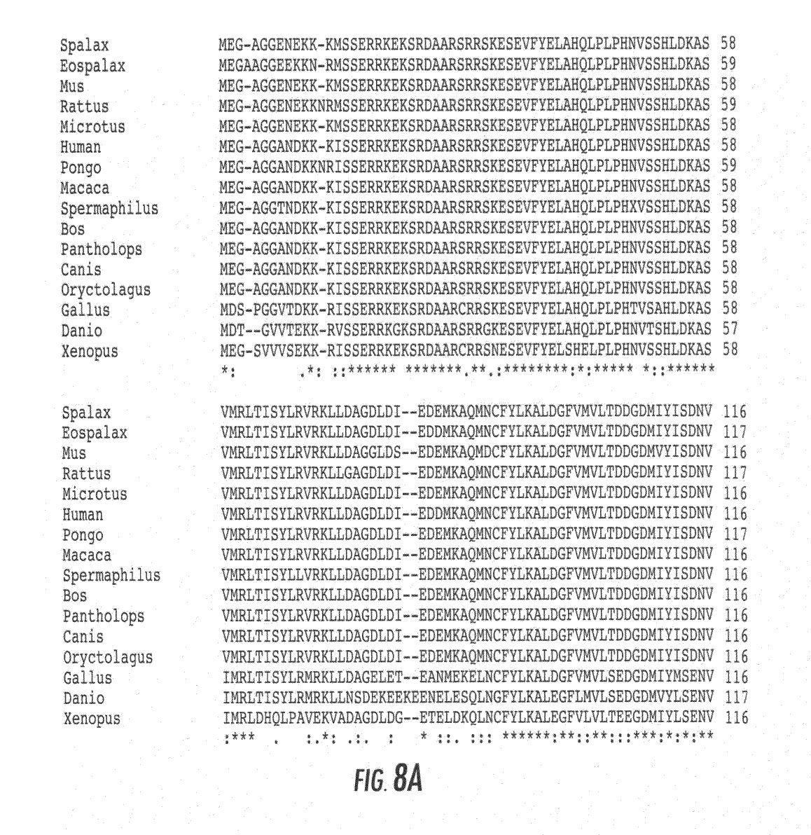

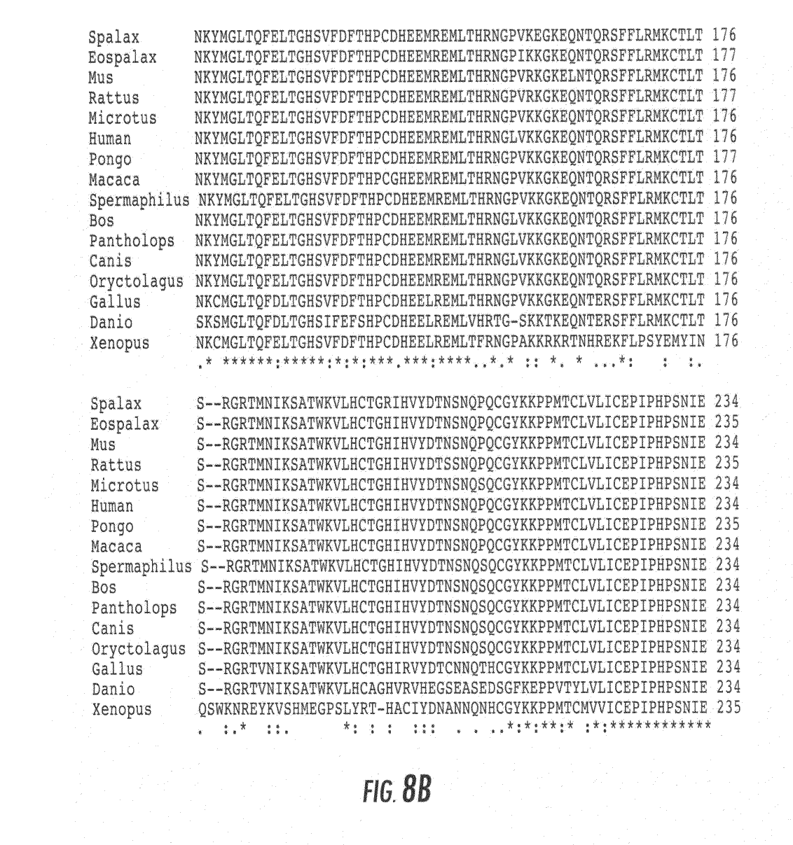

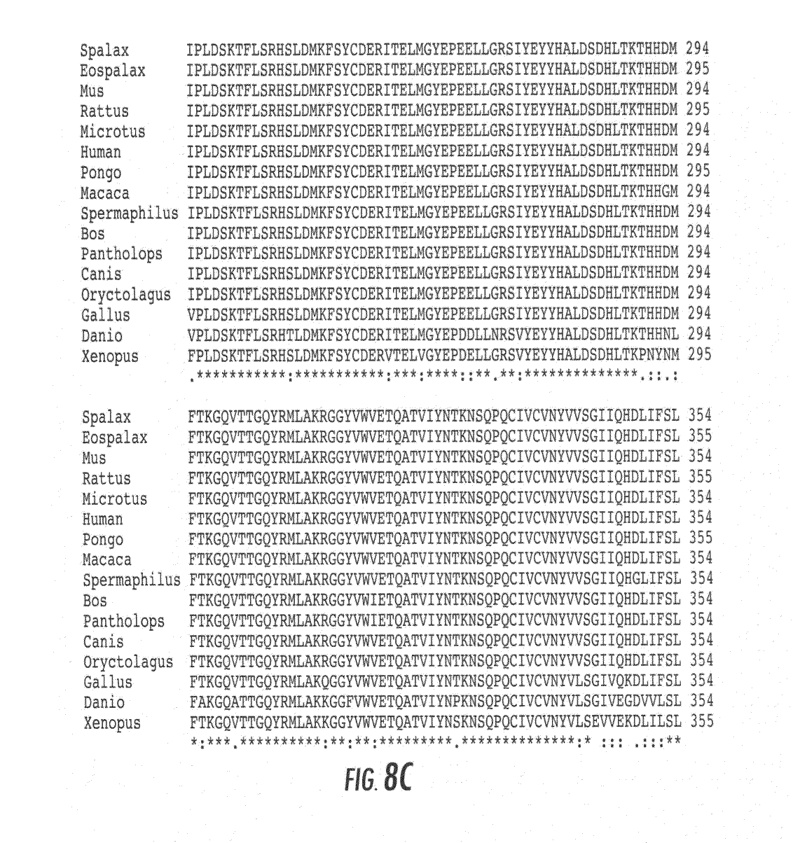

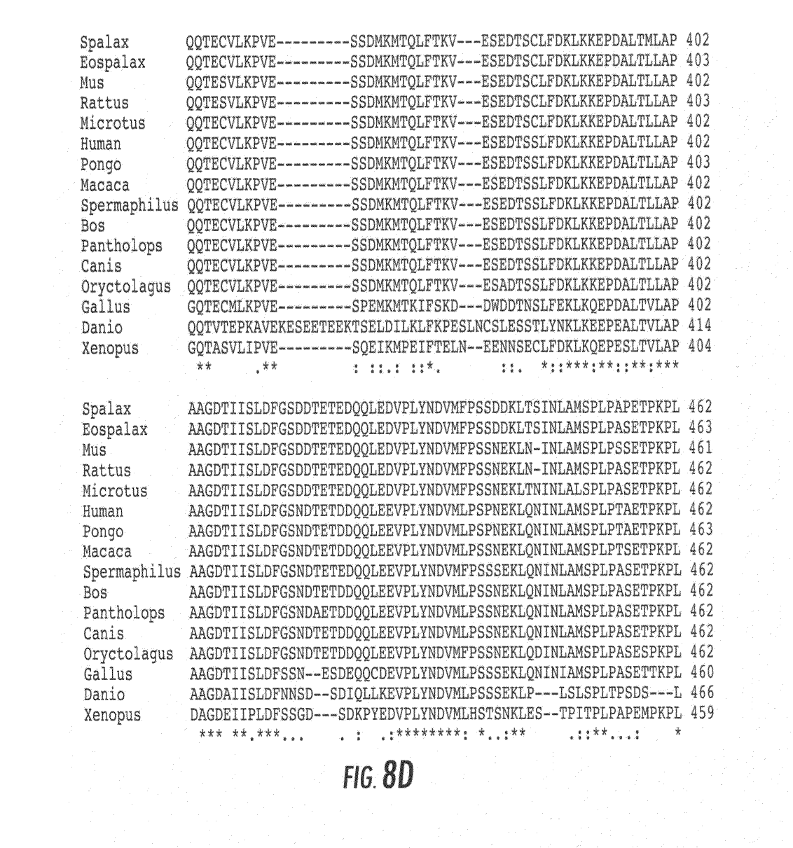

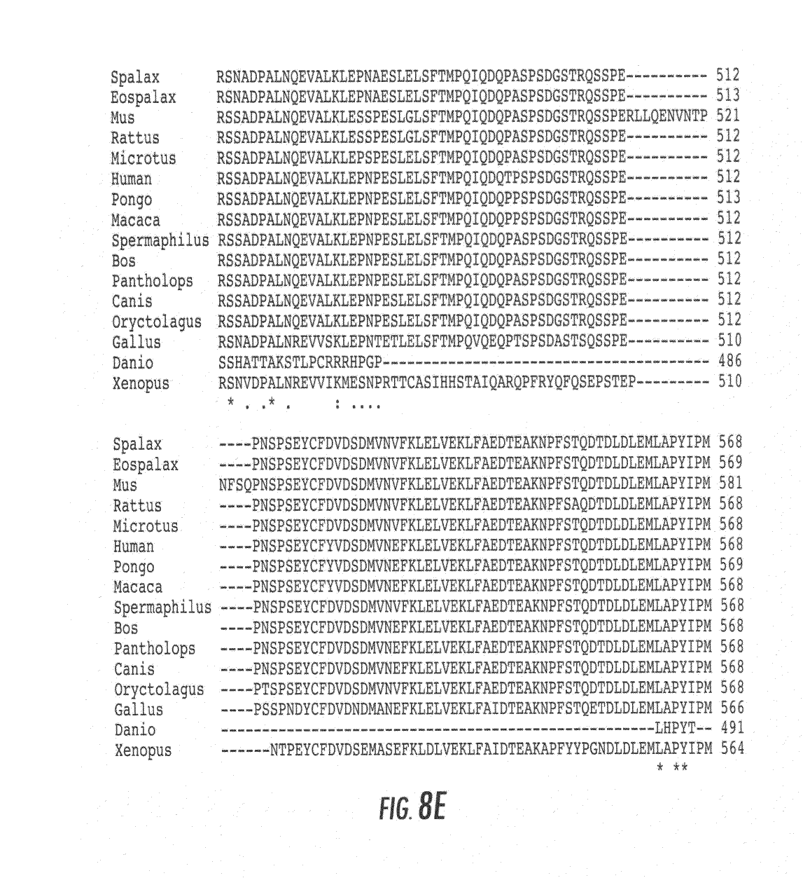

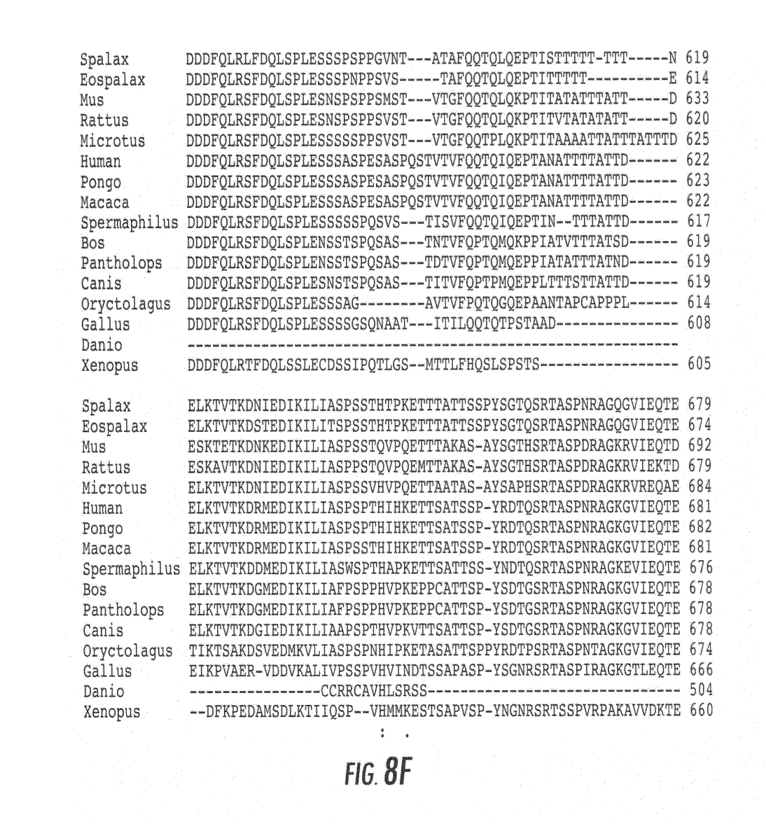

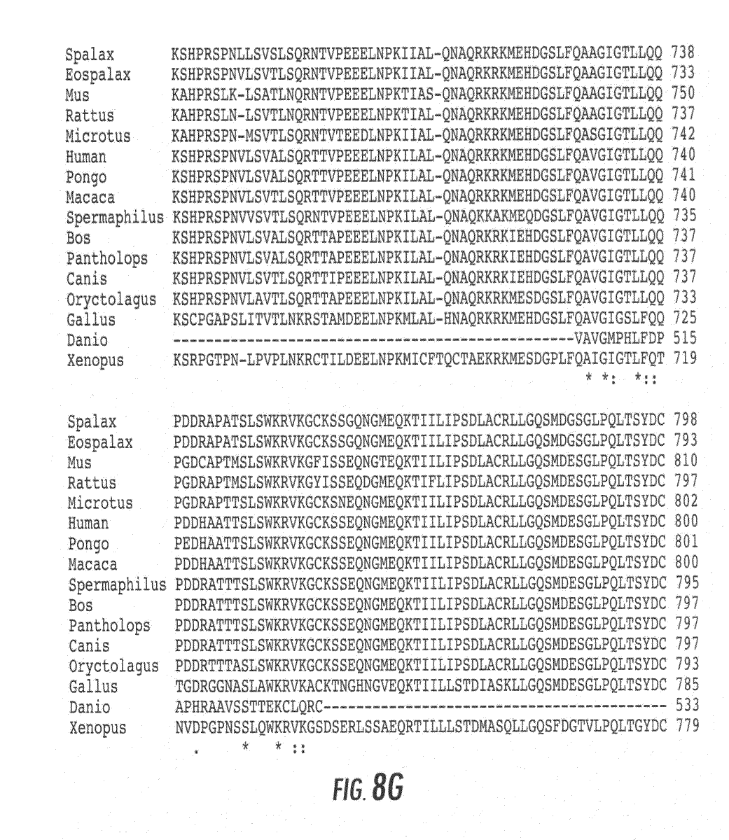

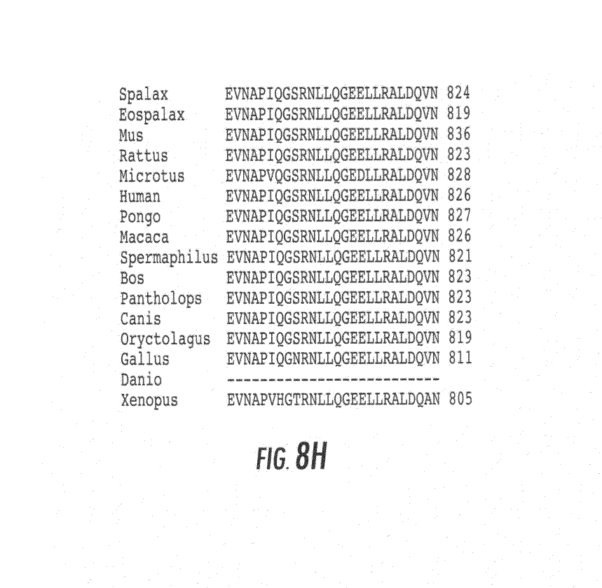

As HIF-1α has been shown to be a key tumor survival factor during cancer therapy, it was postulated that the inhibition of HIF-1α activation through the prevention of NO production would have anti-tumor efficacy. To examine this hypothesis, tumor growth delay experiments with co-administration of radiotherapy (3×6 Gy) and L-NAME were performed in two aggressive tumor models: 4T1 (murine mammary adenocarcinoma; see FIG. 7A) and B16F10 (murine melanoma; see FIG. 7B). In both models, the inhibition of NO production by L-NAME significantly enhanced the therapeutic efficacy of radiotherapy. In addition, the use of L-NAME in conjunction with radiotherapy significantly reduced tumor vasculature (see FIG. 7C).

These results suggested that NO-mediated HIF-1α activation indeed played a critical role in overall tumor response to radiotherapy, consistent with previous reports that the survival of tumor vasculature is key to tumor survival during radiotherapy (Garcia-Barros et al., 2003; Moeller et al., 2004). They further suggested that NOS inhibitors can be used as therapeutic agents to enhance the efficacy of conventional cancer treatments.

### Discussion of Examples 1-7

Understanding HIF-1 regulation during cancer treatment can provide insights into how tumor responds to therapy. This is because HIF-1 has been shown to be a key tumor survival factor after cancer therapy(Moeller et al., 2004; Zhang et al., 2004b). The presently disclosed discovery of HIF-1α upregulation through NO generated from tumor-associated macrophages (FIG. 20) is important for at least two reasons: recognizing the tumor-associated macrophages (TAMs) as a major regulator of HIF-1 and the identification of S-nitrosylation of C533 (human equivalent C520) as a key mechanism for NO-mediated HIF-1α stabilization. The present disclosure establishes for the first time that TAM is a pivotal mediator of tumor angiogenic activity after radiotherapy while the latter unveils a novel mechanism for HIF-1α regulation.

The presently disclosed discovery that NO can regulate HIF-1α stability through S-nitrosylation of Cys533 provides a third avenue for NO-mediated increase in HIF-1 transcriptional activity. It also provides a remarkable example where targeted S-nitrosyaltion of a single cysteine residue in a protein can significantly influence its interaction with other protein(s), very similar to a recent report (Kim et al., 2005) on nitric oxide regulation of the COX-2 gene activation.

Of special interest is the fact that HIF-1 has also been known to enhance iNOS gene expression in a variety of cell types (Jung et al., 2000; Matrone et al., 2004; Melillo et al., 1997). Therefore, it is possible that activated iNOS and HIF-1 forms an amplification loop during wound healing or inflammation. Inconsistent with this hypothesis is a recent report that indicate HIF-1 and iNOS do appear to regulate each other positively under normoxic conditions during bacterial infections (Peyssonnaux et al., 2005). This amplification loop might be a key mechanism during inflammatory response. If true, the relationship between NO and HIF-1α might afford new opportunities of drug development for various inflammatory diseases.

In terms of cancer therapy, the recognition that NO mediated S-nitrosylation of Cys 533 can upregulate HIF-1 activity during radiation or chemotherapy has important implications as well. This is because quite a few studies have indicated that HIF-1 plays critical roles for tumor growth and survival during cancer therapy (Moeller et al., 2004; Yeo et al., 2003). The recognition of the role of NO in the up-regulation of HIF-1α during cancer therapy suggests a promising strategy to enhance current therapy: the use of NOS inhibitors in conjunction with conventional radiation and chemotherapy modalities. The results presented herein combining NOS inhibitor L-NAME and radiotherapy (see FIG. 7) support for this notion. A similar experiment with 4T1 tumors treated with cyclophosphamide (see FIG. 9) suggests that NOS inhibitors can also augment chemotherapy.

Although the presented studies were primarily conducted in tumors that were exposed to ionizing radiation, the same NO-mediated HIF-1 activation pathway might operate in other normal cells/tissues. Indeed, the instant co-inventors have observed NO-mediated HIF-1α activation in macrophages, fibroblasts, and epithelial cells, indicating the general applicability of this pathway.

In summary, the results presented herein establish the importance of nitric oxide-mediated S-nitrosylation in regulating the stability of HIF-1α. They indicate that S-nitrosylation of Cys533 (murine equivalent of human Cys520) in HIF-1α is directly responsible for radiation-induced HIF-1α stabilization in tumors. The instant disclosure also indicates that modulating HIF-1α activation through NOS inhibitors is a promising strategy for therapeutic development in a variety of diseases such as cancer and inflammatory diseases where it has been established that both NO and HIF-1α play prominent roles.

### Materials and Methods Used in the Examples 8-11

Cell Culture.

4T1ODD-luc mouse mammary carcinoma cells and human MCF-7 breast cancer cells were cultured with Dulbecco's modified Eagle's medium (DMEM) containing 10% fetal bovine serum (FBS) and 1% antibiotic-antimycotic solution (GIBCO, Carlsbad, Calif., United States of America). 4T1 ODD-luc is an established 4T1 cell line with a firefly luciferase reporter gene fused with the ODD domain of HIF-1α (Li et al., 2007). 0.5×10⁶ cells were plated into each 10 cm cell culture dish and cultured overnight. Cells were washed with PBS the next day and cultured in either the medium containing equal amount of doxorubicin or the control vehicle (PBS).

Bioluminescent Imaging.

HIF-1α ODD-luciferase reporter activity was detected and quantified by the Xenogen IVIS bioluminescence imaging system (Xenogen Corporation, Alameda, Calif., United States of America) as previously described (Li et al., 2007). For in vitro imaging of cultured cells, 1×10⁵ 4 T1ODD-luc cells per well of 12-well plates were cultured with 2 ml culture medium for 24 hours. Then the cells were treated with doxorubicin hydrochloride (0.1, 1, or 10 μg/ml, Bedford Laboratories, Bedford, Ohio, United States of America) or an equal volume of control vehicle for 3 days under normoxic conditions (21% O2, 5% CO2). In the L-NAME study, 1×10⁵ 4 T1ODD-luc cells were treated with 0.1, 1, or 10 mM L-NAME (Sigma-Aldrich, St. Louis, Mo., United States of America) or control vehicle in the present or absence of 1 μg/ml doxorubicin for 3 days. The activity of HIF-1α luciferase reporter was quantified daily. In animal studies, the bioluminescence of each tumor was detected on day 0 (before doxorubicin treatment) and on post-treatment days 1-7, 10, 13, and 16. During each imaging session mice were anesthetized with inhaled isoflurane and imaged 10 minutes after intraperitoneal (i.p.) injection of luciferin (150 mg/kg). Tumor bioluminescence intensity was normalized to the tumor volume at each time point.

Animal Studies.

5×10⁶ 4 T1ODD-luc tumor cells in 200 μl PBS were injected orthotopically in the right thoracic mammary fat pad of female NCr/nu nude mice (Moeller et al., 2004). Tumor volumes were calculated as follows: volume=(length×width²×π)/6. When tumor size reached 7 mm in diameter, animals were randomized into two groups: the control mice were injected with 100 μl saline and the treated mice were injected once with the maximal tolerated dose (MTD) of doxorubicin (10 mg/kg) via tail vein (Vanhoefer et al., 1997). Tumor volume and luciferase reporter activity in vivo were measured on days 0, 1-7, 10, 13 and 16.

ELISA.

1×10⁵ 4 T1ODD-luc cells per well of 12-well plate were cultured with 1 ml cell culture medium overnight. The cells were treated with 0.1, 1, or 10 μg/ml of doxorubicin or without doxorubicin for 24, 48, and 72 hours. Secreted mouse VEGF in the conditioned medium at each time point was quantified by mouse VEGF ELISA kit (R & D Systems, Minneapolis, Minn., United States of America) and normalized to the number of living cells.

Real-Time PCR.

Total RNA was prepared using the miRVana extraction kit (Applied Biosystems, Austin, Tex., United States of America). 1 μg of total RNA was reverse transcribed into cDNA using the iScript cDNA synthesis kit (BioRad, Hercules, Calif., United States of America). Real-time PCR was performed on an ABI7900HT Fast Real-Time PCR System using Power SYBRGreen PCR Mix (Applied Biosystems). PCR products were verified by melting curves. The threshold cycle (CT) values for each gene were normalized to expression levels of β-actin. The primers used were as follows: mouse iNOS, forward 5′-CTGTGAGACCTTTGATGTCCGAAG-3′ (SEQ ID NO: 22); reverse 5′-CTGGATGAGCCTATATTGCTGTGG-3′ (SEQ ID NO: 23); the same β-actin primers were used for both human and mouse cell lines, forward 5′-GATTACTGCTCTGGCTCCTAGC-3′ (SEQ ID NO: 24); and reverse 5′-GACTCATCGTACTCCTGCTTGC-3′ (SEQ ID NO: 25).

Western Blotting.

Cell nuclear proteins were extracted using the NucBuster protein extraction kit (Novagen, Gibbstown, N.J., United States of America). Total cell lysates or cell nuclear extractions were quantified using a protein assay reagent (Bio-Rad). Equal amounts of protein sample were separated by Bis-HCl gel (Bio-Rad) and transferred to a 0.2 μM Immun-Blot PVDF membrane (Bio-Rad). The antibodies used were as follows: monoclonal mouse anti-HIF-1α antibody (NB100-105, 1:500 dilution, Novus Biologicals, Littleton, Colo., United States of America); rabbit polyclonal anti-HIF-1α antibody (NB100-654, 1:500 dilution, Novus Biologicals); mouse anti-α-tubulin antibody (1:10000 dilution, Sigma-Aldrich); monoclonal mouse anti-β-actin antibody (A2228, 1:5000 dilution, Sigma-Aldrich); polyclonal rabbit anti-iNOS antibody (1:500 dilution, Assay Designs, Ann Arbor, Mich., United States of America); polyclonal sheep anti-histone H1 antibody (NB100-748, 1:500 dilution, Novus); and rabbit anti-STAT1, rabbit anti-phosphor-STAT1 (Tyr701), and rabbit anti-phosphor-STAT1 (Ser727) antibodies (1:1000 dilution, Cell Signaling Technology, Danvers, Mass., United States of America).

Immunohistochemical Staining.

Five mice from a doxorubicin-treated group or a control group that received saline were sacrificed on days 0, 1, 4, 7 and 16. The hypoxia marker pimonidazole (70 mg/kg i.p., NPI, Raleigh, N.C., United States of America) and the perfusion marker dye Hoechst 33342 (1 mg/mouse i.v., Sigma-Aldrich) were administered as previously described (Cao et al., 2005). Upon excision, tumors were snap-frozen in liquid nitrogen, stored at −80° C., and cut into 10 μm frozen sections. HIF-1α fluorescent staining was performed with anti-HIF-1α antibody (Novus). Direct pimonidazole labeling was performed using Hypoxyprobe (mouse monoclonal IgG1, NPI, Raleigh, N.C., United States of America) and Zenon Alexa Fluor 555 mouse IgG labeling kit (Invitrogen). Entire tumor sections were imaged by scanning fluorescent microscopy. Individual scanned images were then reconstructed into one composite image of the entire section by Metamorph. Tumor vasculature was stained by fluorescein-labeled Griffonia simplicifolia lectin I (isolectin B4) (1:200 dilution, Vector Laboratories, Burlingame, Calif., United States of America) as previously described (Cao et al., 2007). Eight random fields (10×) per frozen section were analyzed to determine the mean tumor vascular fraction as the percentage of vessel area in total tumor area in each field. Cell proliferation was determined by immunostaining with a rabbit polyclonal anti-Ki67 antibody (ab15580, 1:100 dilution, Abcam, Cambridge, Massachusettes, United States of America) and an Alexa 594-conjugated goat anti-rabbit IgG antibody (1:300 dilution, Invitrogen). Tumor activated macrophages were stained with rat anti-mouse CD68 antibody (1:100 dilution, Serotec, Raleigh, N.C., United States of America) and an Alexa 488-conjugated anti-rat IgG antibody (1:200 dilution, Invitrogen).

Fluorescent-Activated Cell Sorting (FACS) Analysis.

To detect intracellular nitric oxide levels, 1×10⁶ 4 T1ODD-luc cells were plated per 10-cm cell culture dish with 8 ml of cell culture medium. Doxorubicin (1 μg/ml and 5 μg/ml) or an equal volume of control solvent was added into the cell culture medium the next day and treatment continued for 48 hours. To detect the effects of 1400W (Cayman Chemical, Ann Arbor, Mich., United States of America) and EGCG (Cayman Chemical) on nitric oxide synthesis, 1×10⁶ 4 T1ODD-luc cells were treated with 10 μg/ml 1400W or 15 μM EGCG with or without 1 μg/ml doxorubicin for 48 hours. Equal amounts of solvent were added for the control group. To quantify intracellular nitric oxide, a single cell suspension was made after trypsinization. 2 ml of 5 uM nitric oxide-specific probe DAF-FM diacetate (4-amino-5-methylamino-2′,7′-difluorofluorescein diacetate) (Invitrogen) were incubated with each cell sample at room temperature in darkness for 30 minutes. The cells were washed with PBS and centrifuged to remove the excess DAF-FM diacetate probe. The cells were resuspended into 500 ul of phenol-free DMEM and incubated for another 20 minutes for complete intracellular deacetylation of the probe. The mean fluorescence intensity of the deacetylated DAF-FM reacted with nitric oxide was quantified by FACSCalibur flow cytometer in FITC channel (Becton Dickinson, Franklin Lakes, N.J., United States of America).

Cell Viability Assay.

3×10³ 4 T1ODD-luc cells per well of 96-well plates were cultured with 100 μl of cell culture medium overnight and treated with the control vehicle (PBS), 10 μg/ml 1400W, or 15 μM EGCG, with or without 1 μg/ml doxorubicin on day 0. Cell viability was quantified by Cell Titer-Glo luminescent cell viability assay (Promega, Madison, Wis., United States of America) daily until day 3. The daily cell viability was normalized to the initial cell viability on day 0 within each group.

Statistics.

Groups were first tested for normality and variance homogeneity. Student's t-test was applied for two-group comparison. One-way ANOVA with Student-Newman-Keuls analysis was applied for pairwise multiple comparisons. Difference was considered significant when P<0.05.

### Example 8

### Doxorubicin Treatment Upregulates HIF-1 Expression Both In Vitro and In Vivo

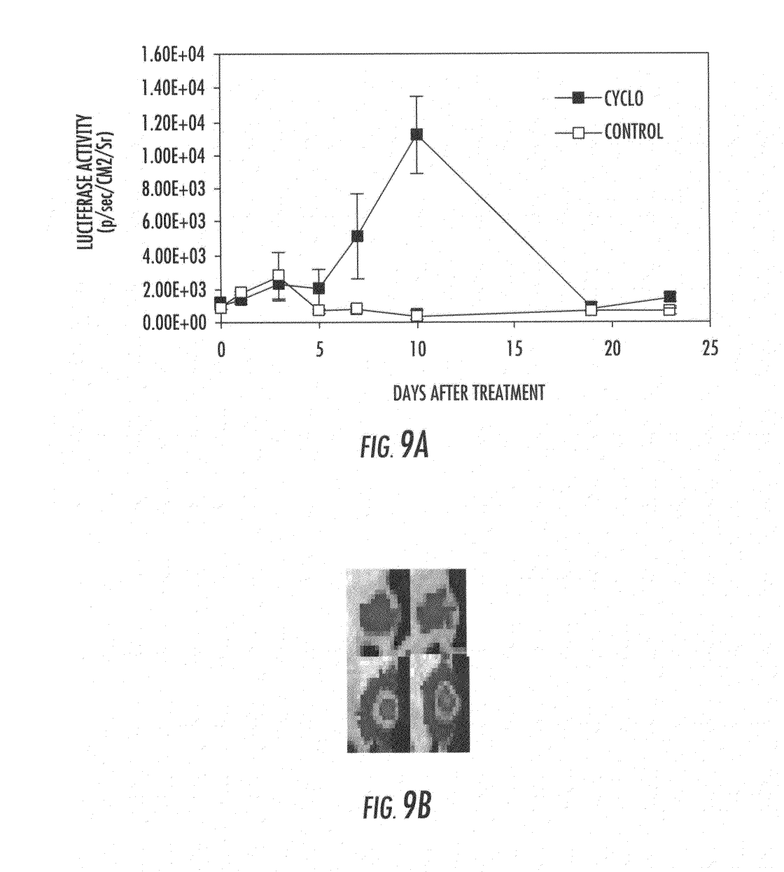

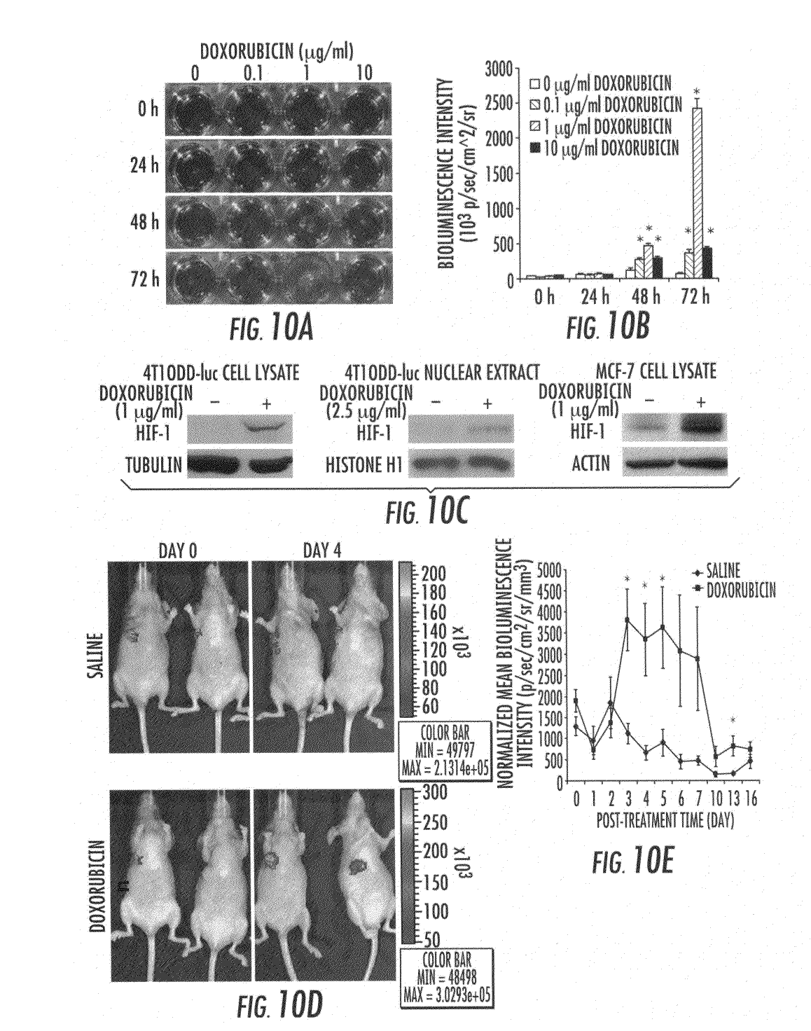

To determine whether doxorubicin affects HIF-1α expression in tumor cells under normoxic conditions, 4T1ODD-luc cells were treated with doxorubicin (0, 0.1, 1, or 10 μg/ml) under normoxic conditions for up to three days. Doxorubicin caused a significant increase in HIF-1α reporter activity 48 hours post-treatment (FIGS. 10A and 10B). 1 μg/ml doxorubicin treatment induced the most potent upregulation of HIF-1α reporter activity. To confirm this doxorubicin-induced normoxic HIF-1α stabilization, Western blotting was performed to directly detect HIF-1α protein expression. Western blotting demonstrated that HIF-1α protein expression was enhanced in both total cell lysate and nuclear extraction in 4T1ODD-luc cells 48 hours post-treatment (FIG. 10C). Doxorubicin treatment also led to normoxic HIF-1α accumulation in MCF-7 human breast cancer cells (FIG. 10C).

To further determine whether in vivo doxorubicin treatment might upregulate tumor HIF-1α expression, doxorubicin was intravenously injected into nude mice bearing orthotopic 4T1ODD-luc tumors. The dynamic time course of HIF-1α reporter activity was non-invasively observed by bioluminescent imaging at multiple time points. Quantitative analysis of bioluminescent images revealed that doxorubicin treatment significantly elevated the HIF-1α reporter activity as early as 3 days post-treatment (FIGS. 10D and 10E). The elevated HIF-1α reporter activity continued for several days until it finally fell to a level similar to that in the control tumors on day 16.

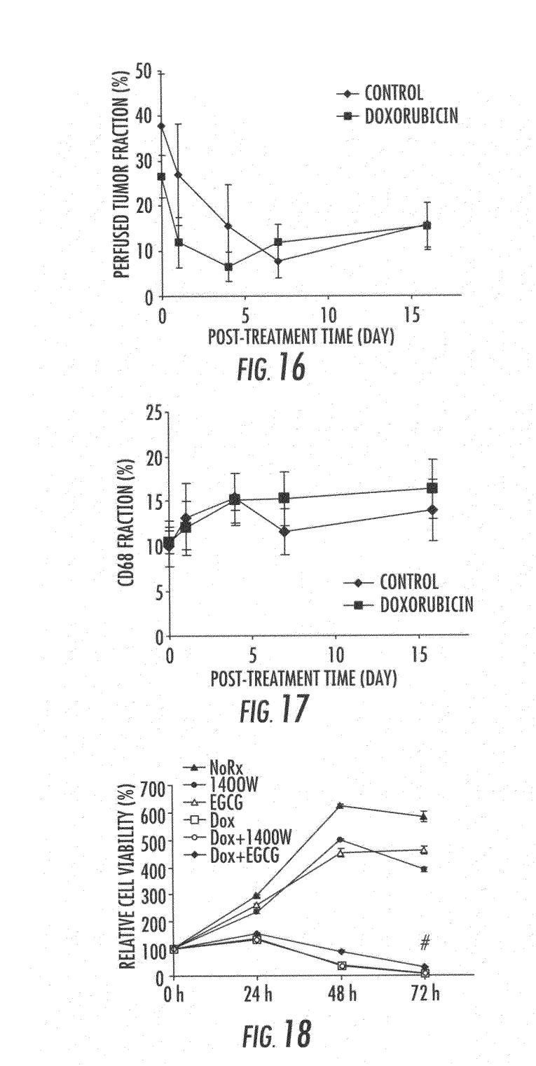

Because low tumor perfusion or tumor hypoxia stabilizes HIF-1α, measures were taken to identify whether the above doxorubicin-induced in vivo HIF-1α upregulation was caused by decreased perfusion or increased hypoxia in post-treatment tumors. The immunohistochemical staining of HIF-1α protein, hypoxic marker pimonidazole, and the tissue labeling of perfusion dye Hoechst 33342 were compared between the doxorubicin-treated tumors and the saline-treated control tumors on post-treatment days 1, 4, 7, and 16. There was no difference in tumor HIF-1α positive fraction between the two groups before doxorubicin treatment (on day 0). Doxorubicin chemotherapy significantly increased the HIF-1α fraction when compared to the control saline treatment on post-treatment days 1, 4, and 7 (FIGS. 11A and 11B). In contrast, there was no significant difference in either hypoxic tumor fraction as shown by pimonidazole staining or in perfused tumor fraction as shown by Hoechst 33342 labeling between the doxorubicin-treated tumors and the control tumors (FIGS. 11C and 16). These results suggest that the doxorubicin-induced in vivo upregulation of tumor HIF-1α was not caused by increased tumor hypoxia or decreased tumor perfusion.

### Example 9

### Doxorubicin-Induced HIF-1α Upregulation Leads to Increased VEGF Secretion by Tumor Cells In Vitro and Resurgent Post-Treatment Tumor Angiogenesis In Vivo

Clinical studies have suggested that tumor vascular index rather than tumor size is the most reliable prognostic indicator for tumor relapse after chemotherapy (Gasparini et al., 2001). One important consequence of HIF-1α stabilization is to promote angiogenesis by stimulating multiple downstream angiogenic cytokines (Semenza, 2003; Moeller et al., 2004; Carmeliet et al., 1998; Maxwell et al., 1997).

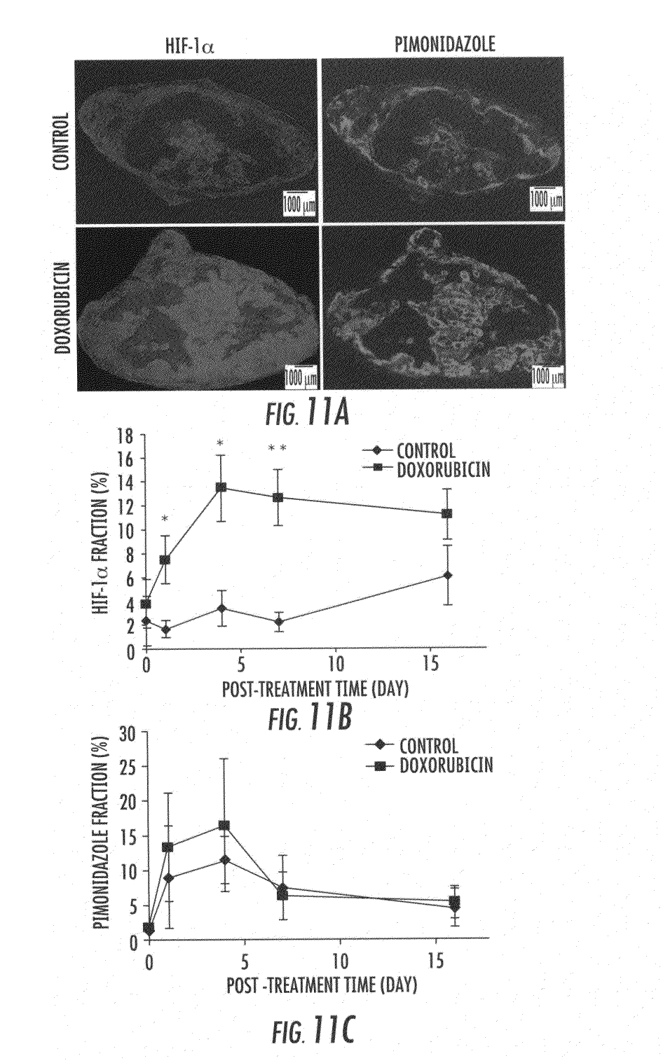

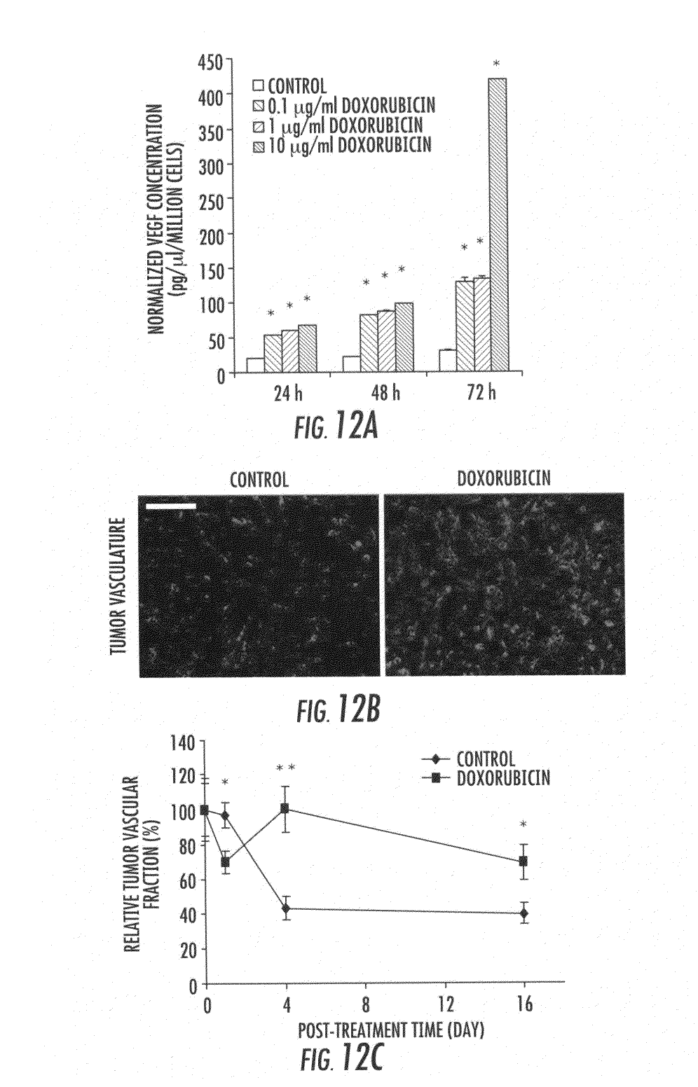

In this Example the effect of doxorubicin chemotherapy on tumor cell VEGF secretion and tumor angiogenesis was determined. The ELISA results demonstrated that serial concentrations of doxorubicin treatments (0.1, 1, and 10 μg/ml) significantly elevated the secreted VEGF level by 4T1ODD-luc tumor cells under normoxic conditions in vitro (FIG. 12A). Fluorescent staining of the tumor vasculature demonstrated more tumor vessels in the doxorubicin-treated tumors than the saline-treated control tumors on day 4 post-treatment (FIG. 12B). To compare tumor angiogenesis between the doxorubicin treated and the control saline treated groups, the relative change in tumor vasculature fraction on post-treatment days 1, 4, and 16 was quantified, which was then normalized to the initial tumor vascular fraction before treatment (on day 0) (FIG. 12C). An advantage of the relative change of tumor vascular fraction is it identifies the dynamic fluctuation between angiogenesis and vascular destruction. It also serves as a sensitive indicator to reveal the turning points in a trend of either vascular formation or destruction. Doxorubicin treatment caused a significant decrease in tumor vascular fraction compared to the control treatment during the first 24 hours (FIG. 12C). However, this downtrend in tumor angiogenesis was immediately reversed by a potent resurgence of angiogenesis in doxorubicin-treated tumors (FIG. 12C). This resurgent tumor angiogenesis continued until it peaked on post-treatment day 4, when the mean relative tumor vascular fraction in doxorubicin-treated tumors was even higher than before chemotherapy treatment. After day 4 the relative tumor vascular fraction in doxorubicin-treated tumors began a sustained downward trend, but was still significantly higher compared to the saline-treated tumors on day 16. In contrast, the relative tumor vascular fraction in the saline-treated tumors progressively decreased with continued tumor growth, and on day 16 was at a significantly lower level than in the doxorubicin-treated tumors.

### Example 10

### Nitric Oxide (NO) and Nitric Oxide Synthase (NOS) Play Roles in Doxorubicin-Induced Normoxic HIF-1α Stabilization

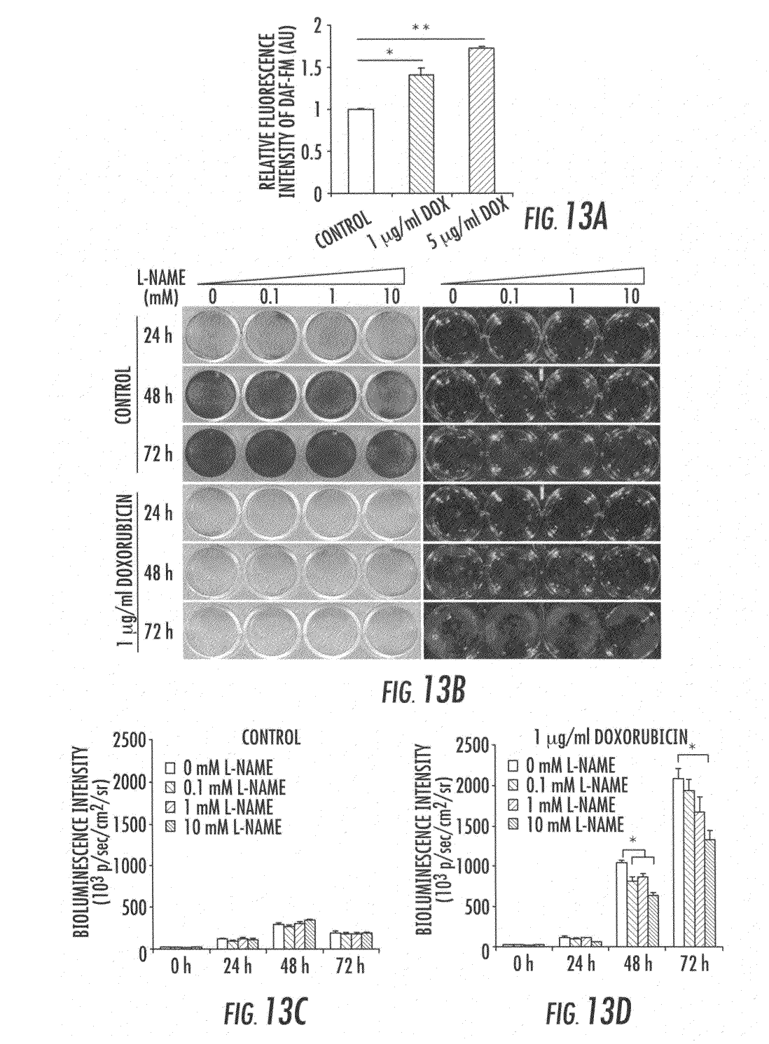

Because the above results suggest that hypoxia is not the cause for doxorubicin-induced HIF-1α stabilization, measures were taken to determine the factors that might upregulate HIF-1α and VEGF under normoxic conditions. Specifically, experiments were designed to determine whether doxorubicin would increase intracellular NO levels in the 4T1ODD-luc cells. By the use of DAF-FM diacetate (a NO-specific indicator), increased intracellular NO levels were detected compared to control at 48 hours following treatment with 1 μg/ml and 5 μg/ml doxorubicin (FIG. 13A). To identify whether nitric oxide synthase was the cause for doxorubicin-induced HIF-1α upregulation, 4T1ODD-luc cells were treated with serial concentrations of the nitric oxide synthase inhibitor L-NAME (0, 0.1, 1, and 10 μg/ml) in the presence or absence of doxorubicin (1 μg/ml). Treatment continued for 72 hours, during which time both HIF-1α reporter activity and cell viability were assessed daily (FIG. 13B). In the absence of L-NAME, doxorubicin significantly increased HIF-1α reporter activity while killing many 4T1ODD-luc cells (FIGS. 13A-13C), which was the same as shown above (FIG. 10A). In the absence of doxorubicin, increased dose of L-NAME did not change the basal level of HIF-1α reporter activity (FIGS. 13B and 13C). However, when combined with doxorubicin treatment, L-NAME caused significant dose-dependent inhibition of doxorubicin-induced HIF-1α upregulation (FIGS. 13B and 13D). Therefore, these results suggest that nitric oxide synthase participates in doxorubicin-induced normoxic HIF-1α stabilization.

### Example 11

### Doxorubicin Upregulates Normoxic HIF-1α Expression by Activating the STAT1/Inducible Nitric Oxide Synthase (iNOS) Signaling Pathway

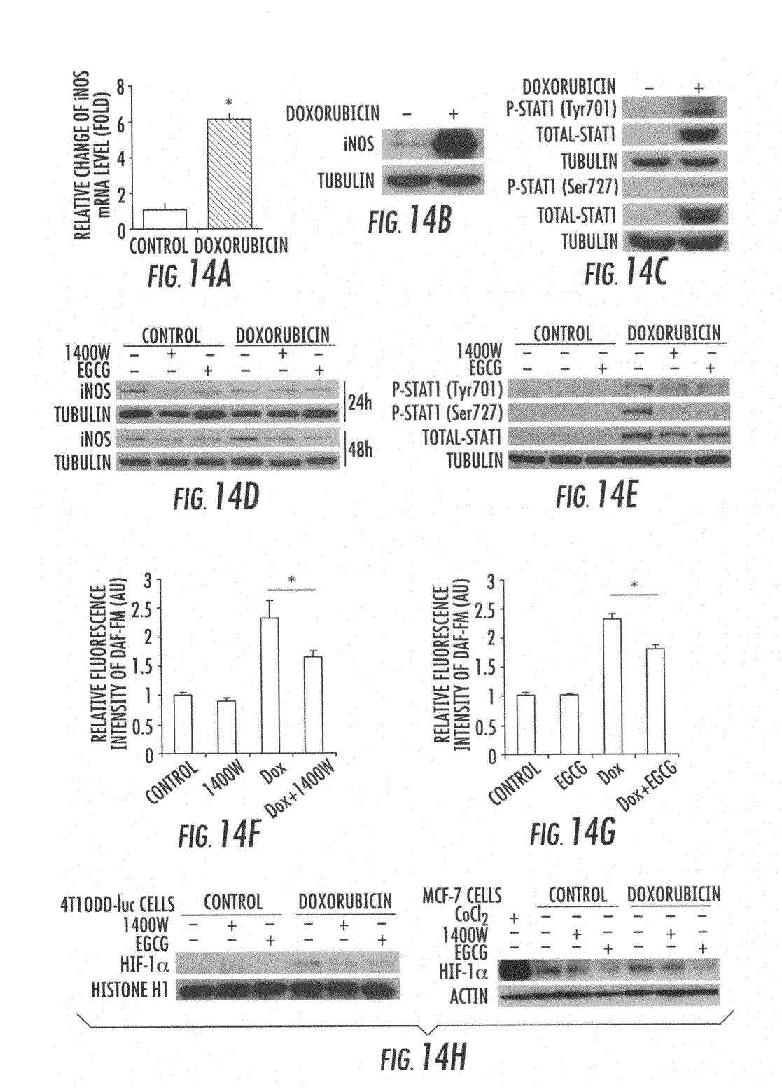

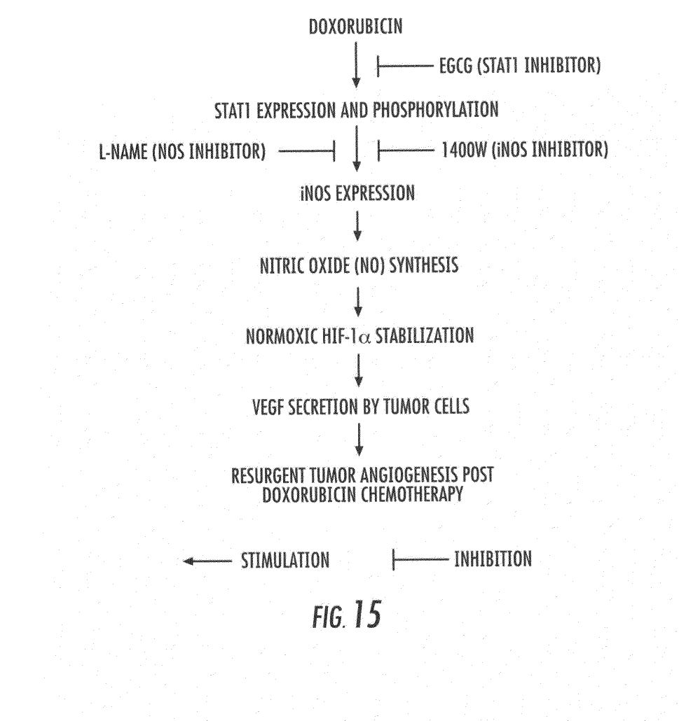

Nitric oxide is synthesized by three isoforms of nitric oxide synthase: inducible (iNOS), neuronal (nNOS), and endothelial (eNOS). Inducible NOS (iNOS) is the isoenzyme most commonly associated with carcinogenesis and tumor progression (Lechner et al., 2005). Experiments were conducted to investigate whether iNOS plays a crucial role in doxorubicin-induced normoxic HIF-1α upregulation. First, the effects of doxorubicin and control treatment on the iNOS mRNA level in 4T1ODD-luc cells were compared. Quantitative real-time PCR demonstrated that doxorubicin significantly enhanced iNOS transcription compared to the control treatment (FIG. 14A). Western blotting further confirmed that doxorubicin stimulated iNOS expression under normoxic conditions (FIG. 14B).

Steps were then taken to identify the upstream regulatory molecule enhancing iNOS expression in response to doxorubicin treatment. Signal transducer and activator of transcription 1 (STAT1) is a transcription factor required for iNOS transcription and activation (Samardzic et al., 2001; Guo et al., 2008). Western blotting revealed that doxorubicin treatment not only upregulated the expression of STAT1 but also promoted the activation of STAT1 through the phosphorylation of its two residues—Tyr701 and Ser727 (FIG. 14C).

To further confirm the role of the STAT1/iNOS signaling pathway in normoxic HIF-1α accumulation, it was sought to determine whether inhibition of iNOS or STAT1 would suppress doxorubicin-induced HIF-1α upregulation. To this end, 4T1ODD-luc cells were individually treated with the specific inhibitor of either iNOS or STAT1 (1400W and epigallocatechin gallate [EGCG], respectively), with or without doxorubicin. Without doxorubicin treatment, both 1400W and EGCG suppressed the basal level of iNOS expression in tumor cells (FIG. 14D, 24 h and 48 h). Because doxorubicin alone caused an initial decrease in iNOS expression during the first 24 hours post-treatment (FIG. 14D, 24 h), the inhibition of iNOS expression by either 1400W or EGCG in the doxorubicin-treated cells was not obvious during this time window (FIG. 14D, 24 h). In contrast, doxorubicin treatment caused an increase in iNOS expression in 4T1ODD-luc tumor cells by 48 hours post-treatment, suggesting that the doxorubicin-induced upregulation of iNOS expression occurs between 24 and 48 hours post-treatment (FIG. 14D, 48 h). During this time period both 1400W and EGCG successfully suppressed the doxorubicin-induced iNOS upregulation (FIG. 14D, 48 h). Western blotting also confirmed that 1400W and EGCG potently suppressed the doxorubicin-induced upregulation of STAT1 expression and activation by 48 hours post-treatment (FIG. 14E). The down-regulation of STAT1 expression and activation by iNOS-specific inhibitor 1400W indicates that iNOS may participate in a positive feedback cycle stimulating STAT1 signaling. Flow cytometry analysis confirmed that both 1400W and EGCG significantly suppressed the doxorubicin-induced increase of intracellular NO 48 hours post-treatment due to the inhibition of iNOS (FIGS. 14F and 14G).

Finally, both 1400W and EGCG successfully suppressed the doxorubicin-induced normoxic HIF-1α accumulation in 4T1ODD-luc cells on post-treatment day 2, as well as in MCF-7 cells on post-treatment day 3. (FIG. 14H). These results support the conclusion that iNOS and STAT1 are rational targets for preventing doxorubicin-induced normoxic HIF-1α accumulation. Because the complex roles of iNOS and STAT1 include stimulating apoptosis under certain conditions (Iwashina et al., 1998; Thomas et al., 2004), additional caution was taken to evaluate whether combining 1400W or EGCG with doxorubicin might affect the cell killing by doxorubicin. Cell viability assay revealed that the above combined treatments did not attenuate the cytotoxicity of doxorubicin (FIG. 18) while suppressing doxorubicin-induced normoxic HIF-1α accumulation.

### Discussion of Examples 8-11

There has been intense interest in developing novel therapeutic strategies to target HIF-1α in cancer therapy for three main reasons. First, HIF-la expression has been found in the majority of solid tumors, while it is usually absent in normal tissues (Talks et al., 2000). This is because hypoxia, which is a characteristic feature of solid tumors, stabilizes HIF-1α in tumor cells (Wang & Semenza, 1993; Harris, 2000; Giaccia et al., 2003). The differential expression and distribution of HIF-1α between normal tissues and tumors allow the inhibition of HIF-1α to target tumor cells, while sparing normal tissues. Second, because HIF-1 is a regulatory hub for many key aspects in cancer biology such as angiogenesis and therapeutic resistance, inhibition of HIF-1α leads to the disruption of multiple non-overlapping vital mechanisms for tumor progression. Third, inhibition of HIF-1α may exploit tumor hypoxia by converting it from a treatment obstacle into a therapeutic advantage. In addition to hypoxia, many other non-hypoxic factors have also been found to upregulate HIF-1α expression in tumor cells. For examples, both radiotherapy and photodynamic therapy can cause hypoxia-independent upregulation of HIF-1α.

Prior to the instant disclosure no know research addressed whether chemotherapy upregulates HIF-1α expression in tumor cells under normoxic conditions. Yet it is important to address this question, not only because HIF-1α is a major determinant for tumor angiogenesis, therapeutic resistance, and tumor relapse, but also because the majority of chemotherapy drugs exert most of their killing effects on aerobic cells, not hypoxic cells. There are multiple reasons for this, including limited penetration depth of drugs to hypoxic tumor cells, a low proliferative fraction in hypoxic tumor regions, and elevations in drug detoxification pathways, such as glutathione, in hypoxic tumor cells (Brown & Wilson, 2004; Minchinton & Tannock, 2006). Doxorubicin, in particular, does not penetrate into hypoxic tumor regions very effectively (Primeau et al., 2005). Therefore, identification of key molecular mechanisms for normoxic HIF-1α stabilization in tumor cells during chemotherapy can provide important insight into how tumors respond to chemotherapy, obtain growth advantages, and develop therapeutic resistance that promotes tumor relapse.

The instant disclosure, including Examples 8-11, demonstrates the successful identification of the activation of the STAT1/iNOS/NO signaling cascade, which appears to play a dominant role in normoxic HIF-1α stabilization in tumor cells during doxorubicin chemotherapy. The significance of this doxorubicin-induced normoxic HIF-1α accumulation and its underlying mechanism is illustrated below in several ways. First, this study reveals that doxorubicin upregulates the expression and activation of STAT1. STAT1 is an important modulator which potentiates chemotherapy-induced apoptosis (Thomas et al., 2004). It is also an upstream transcription activator for iNOS expression (Samardzic et al., 2001; Guo et al., 2008). NO plays two opposite roles to regulate HIF-1α stability depending on its concentration. Low concentrations of NO (<400 nM) destabilize HIF-1α under hypoxic conditions. However, high concentrations of NO (>1 μM) stabilize HIF-1α under both normoxic and hypoxic conditions.

Relevant here, NO is able to upregulate the expression and activity of HIF-1 under normoxic conditions through three mechanisms: (1) by the use of different NO donors, several groups have demonstrated that NO inhibits normoxic prolyl hydroxylase activity, causing HIF-1α accumulation under normoxic conditions; (2) NO S-nitrosylates the Cys533 in the oxygen-dependent degradation domain of HIF-1α, which prevents normoxic HIF-1α degradation; and (3) NO inhibits FIH enzyme activity, abolishing the FIH-mediated asparagine hydroxylation of HIF-1α. By suppressing FIH, NO thereby promotes the binding between HIF-1α and its coactivator CBP/p300, which activates transcription of HIF-1 downstream genes. In the instant experiments it was found that the upregulated iNOS expression and NO synthesis during doxorubicin treatment led to normoxic HIF-1α accumulation in tumor cells. The temporal course of doxorubicin-induced normoxic HIF-1α accumulation is 3-5 days post-treatment, which is similar to the time when radiotherapy upregulates tumor HIF-1α expression (4-7 days post-treatment).

However, the mechanism of doxorubicin-induced normoxic HIF-1α accumulation differs from that induced by radiotherapy. It was previously shown that the generation of reactive oxygen species and stress granule depolymerization in tumor cells after ionizing radiation cause HIF-1α accumulation and enhanced translation of HIF-1-regulated genes. Ionizing radiation also stimulates tumor associated macrophages (TAMs) to synthesize NO. The TAM-generated exogenous NO then diffuses into tumor cells to S-nitrosylate Cys533 of HIF-1α, causing normoxic HIF-1α stabilization. The absence of TAMs in the in vitro experiments using 4T1ODD-luc and MCF-7 tumor cell lines (FIGS. 10C, 13A-13D, and 14A-14H) and the lack of a significant difference in activated TAM levels between the doxorubicin-treated tumors and the control tumors (FIG. 17) suggest that tumor cells rather than TAMs are the dominant NO producer for the normoxic HIF-1α accumulation in response to doxorubicin chemotherapy.

Second, the presently disclosed subject matter unveils novel hypoxia-independent chemotherapy-induced VEGF secretion and tumor angiogenesis, highlighting the importance of combining anti-VEGF/antiangiogenic therapy with conventional chemotherapy. The presently disclosed subject matter demonstrates that doxorubicin chemotherapy stimulates HIF-1-mediated VEGF secretion (FIG. 12A), causing resurgent angiogenesis between days 1 and 4 after the initial devascularization in doxorubicin-treated tumors (FIG. 12C). In addition, the presently disclosed subject matter also provides new evidence to explain how iNOS promotes tumor angiogenesis. The data disclosed herein reveal that the NO synthesized by iNOS is able to stabilize HIF-1α under normoxic conditions (FIGS. 13A-13D and 14A-14H) and the upregulated HIF-1α promotes VEGF secretion and resurgent tumor angiogenesis (FIGS. 12A-12C). It is known that hypoxia stimulates VEGF expression and angiogenesis through HIF-1α (Maxwell et al., 1997; Forsythe et al., 1996; Bergers et al., 2003). Here the doxorubicin-induced stimulation in normoxic VEGF secretion and resurgent tumor angiogenesis provide new evidence for combining doxorubicin chemotherapy with an anti-VEGF/anti-angiogenic treatment to suppress not only hypoxia-induced but also chemotherapy-induced VEGF upregulation and angiogenesis.

Third, the temporal details of the dynamic alternations in tumor HIF-1α levels, iNOS levels, and angiogenesis elucidated in this study provide important rationales to optimize the timing of combined doxorubicin chemotherapy with HIF-1α inhibitors. Because the doxorubicin-induced upregulation of HIF-1α and iNOS appear between 24 hours and 48 hours post-treatment (FIGS. 10B and 14D), in some embodiments the inhibition of HIF-1α and iNOS should start at the beginning of chemotherapy, or at least no later than 24 hours post-treatment, to maximize the suppression of HIF-1α accumulation. Because the resurgent angiogenesis in doxorubicin-treated tumors is initiated as early as 24 hours post-treatment and is sustained for several days (FIG. 12C), in some embodiments the anti-VEGF/antiangiogenic therapy should start at the beginning or within the first 24 hours after doxorubicin chemotherapy to maximally inhibit the chemotherapy-induced angiogenesis.

Fourth, the presently disclosed subject matter further suggest that the inhibition of doxorubicin-induced normoxic HIF-1α accumulation by STAT1- or iNOS-specific inhibitors does not depend on the expression of the p53 tumor suppressor. Mutations in the p53 tumor suppressor gene are the most common genetic alteration in human cancers (Cho et al., 1994). The 4T1ODD-luc mouse breast tumor line is p53 null (Wang et al., 1998), whereas the MCF-7 human breast cancer cell line expresses a wild-type p53 (Nagasawa et al., 1995). The HIF-1α upregulation in both p53-null and p53 wild-type tumor cell lines indicates that p53 tumor suppressor does not appear to be a determinant in doxorubicin-induced normoxic HIF-1α stabilization (FIG. 10C). In addition, the successful suppression of doxorubicin-induced normoxic HIF-1α accumulation by 1400W and EGCG in both cell lines suggests that the STAT1/iNOS-targeting strategy to inhibit HIF-1α does not depend on p53 expression. Thus, combined doxorubicin chemotherapy with a HIF-1α upstream inhibitor may potentially be applied to treat both p53 wild-type and p53-null tumors.

The above findings further clarify the role of doxorubicin in regulating tumor cell HIF-1α levels at different oxygen tensions. Under normoxic conditions, doxorubicin induces HIF-1α stabilization by activating the STAT1/iNOS/NO signaling pathway. This is in contrast to hypoxic conditions, where doxorubicin inhibits hypoxia-induced HIF-1 transcriptional activity (Lee et al., 2009). The mechanistic elucidation of chemotherapy-induced normoxic HIF-1α accumulation significantly contributes to the current understanding of HIF-1α s a major determinant of tumor progression, which may help to improve conventional cancer therapies by the use of HIF-1 inhibitors. In addition, the presently disclosed subject matter suggests that the combination of HIF-1α inhibitors with conventional chemotherapy (FIG. 15) not only targets hypoxic tumor cells, but also the heretofore overlooked normoxic tumor cells.

### REFERENCES

The references listed below, as well as all references cited in the specification, including patents, patent applications, journal articles, and all database entries (e.g., GENBANK® database entries, including any annotations presented in the databases associated with the disclosed sequences), are incorporated herein by reference to the extent that they supplement, explain, provide a background for, or teach methodology, techniques, and/or compositions employed herein.
Adelman et al. (1983) DNA 2:183-193.
Alam & Cook (1990) Anal Biochem 188:245-254.
Alderton et al. (2001). Biochem J 357:593-615.
Altschul et al. (1990) J Mol Biol 215:403-410.
Apte et al. (2009) J Labelled Compd Rad. 52:S408-S.
Ausubel (1995) Short Protocols in Molecular Biology, 3rd ed. Wiley, New York, New York, United States of America.
Ausubel et al. (1992) Current Protocols in Molecular Biology. Wiley, New York, New York, United States of America.
Barr et al. (2008) Int J Oncol 32: 41-48.
Bass (2001) Nature 411:428-429.
Berchner-Pfannschmidt et al. (2007) J Biol Chem 282: 1788-1796.
Bergers & Benjamin (2003) Nat Rev Cancer 3: 401-410.
Bertout et al. (2008) Nat Rev Cancer 8: 967-975.
Brizel et al. (1996). Cancer Res 56:5347-5350.
Brizel et al. (1999). Radiother Oncol 53:113-117.
Brown & Wilson (2004) Nat Rev Cancer 4: 437-447.
Brune & Zhou (2007) Methods Enzymol 435: 463-478.
Cao et al. (2005). Cancer Res 65: 5498-5505.
Cao et al. (2007) Cancer Res 67: 3835-3844.
Carcereri de Prati et al. (2005) Curr Med Chem 12:1819-1828
Carmeliet et al. (1998). Nature 394: 485-490.
Chan et al. (2002). J Biol Chem 277:40112-40117.
Chen et al. (2003). J Biol Chem 278:13595-13598.
Cho et al. (1994) Science 265: 346-355.
Cipolla et al. (2000) Hum Gene Ther 11:361-371.
Cockman et al. (2000). J Biol Chem 275: 25733-25741.
Colombo & Mantovani (2005). Cancer Res 65:9113-9116.
Comerford et al. (2002) Cancer Res 62: 3387-3394.
Contag et al. (1998). Nat Med 4:245-247.
Cramer et al. (2003). Cell 112:645-657.
Cubitt et al. (1995) Trends Biochem Sci 20:448-455.
Dachs et al. (1997). Nat Med 3: 515-520.
Dales et al. (2005) Int J Cancer 116: 734-739.
Denko, N C (2008). Nat Rev Cancer 8: 705-713.
Dewhirst et al. (2008). Nat Rev Cancer 8: 425-437.
Dorr et al. (1997) Cancer Chemotherapy Handbook, 2d edition, Appleton & Lange, Stamford, Conn., United States of America.
Elbashir et al. (2001) Nature 411:494-498,
Forsythe et al. (1996) Mol Cell Biol 16: 4604-4613.
Fryknas et al. (2007) Int J Cancer 120: 189-195.
Fukuda et al. (2002). J Biol Chem 277:38205-38211.
Fukuda et al. (2003). Cancer Res 63:2330-2334.
Garcia-Barros et al. (2003). Science 300:1155-1159.
Gasparini et al. (2001) Breast Cancer Res Treat 65: 71-75.
GENBANK® Accession Nos. AAA62405; AAH69465; AAP43517; AAU14021; AAX89137; AAY27087; ABB17537; BAE01417; BC069465; CAB96628; CAG29396; CAH93355; NM_{—}000625; NP_{—}000616; NP_{—}001521; NP_{—}034561; NP_{—}077335; NP_{—}776764; NP_{—}956527; NP_{—}989628; S76748; U17327; U59496; and XP_{—}852278.
Generali et al. (2006). Clin Cancer Res 12: 4562-4568.
Giaccia et al. (2003) Nat Rev Drug Discov 2: 803-811.
Giaccia et al. (2003). Nat Rev Drug Discov 2:803-811.
Glover & Hames (1995) DNA Cloning: A Practical Approach, 2nd ed. IRL Press at Oxford University Press, New York, N.Y., United States of America.
Goldmann et al. (2004). Infect Immun 72:2956-2963.
Greenberg et al. (1994) Mol Endocrinol 8:230-239.
Gruber et al. (2004). Breast Cancer Res 6: R191-198.
Gunning et al. (2007) Bioorg Med Chem Lett 17:1875-1878.
Guo et al. (2008) Surgery 144: 182-188.
Habib et al. (1999) Hum Gene Ther 10:2019-2034.
Hagen et al. (2003). Science 302:1975-1978.
Harris (2002) Nat Rev Cancer 2:38-47.
Harris A L (2002) Nat Rev Cancer 2: 38-47.
Henikoff & Henikoff (1992) Proc Natl Acad Sci USA 89:10915-10919.
Hon et al. (2002). Nature 417: 975-978.
Huang et al. (1999). Nat Biotechnol 17:1033-1035.
Isaacs et al. (2005). Cancer Cell 8:143-153.
Ivan et al. (2001). Science 292: 464-468.
Ivan et al. (2001). Science 292:464-468.
Iwashina et al. (1998) Circulation 98: 1212-1218.
Jaakkola et al. (2001). Science 292: 468-472.
Jaakkola et al. (2001). Science 292:468-472.
Jaffrey & Snyder (2001). Sci STKE 2001.
Jemal et al. (2005) CA Cancer J Clin 55: 10-30.
Jeong et al. (2002). Cell 111: 709-720.
Jeong et al. (2002). Cell 111:709-720.
Jiang et al. (1997). Cancer Res 57:5328-5335.
Jung et al. (2000). Circ Res 86:319-325.
Kamura et al. (2000). Proc Natl Acad Sci USA 97: 10430-10435.
Karlin & Altschul (1993) Proc Natl Acad Sci USA 90:5873-5877.
Keith B, Simon M C (2007). Cell 129: 465-472.
Kim et al. (2005). Science 310:1966-1970.
Kimura et al. (2000) Blood 95: 189-197.
Krishnamachary et al. (2003). Cancer Res 63: 1138-1143.
Kurihara et al. (2000) J Clin Invest 106:763-771.
Lando et al. (2002). Science 295: 858-861.
Lando et al. (2002a). Genes Dev 16:1466-1471.
Lando et al. (2002b). Science 295:858-861.
Laughner et al. (2001) Mol Cell Biol 21:3995-4004.
Lechner et al. (2005) Semin Cancer Biol 15: 277-289.
Lee et al. (2000) Anticancer Res 20:417-422.
Lee et al. (2007) PLoS Med 4: e186.
Lee et al. (2009) Proc Natl Acad Sci USA 106: 2353-2358.
Leek et al. (2000). J Pathol 190:430-436.
Leek et al. (2002). Cancer Res 62:1326-1329.
Leibel & Phillips (1998) Textbook of Radiation Oncology, Saunders, Philadelphia, United States of America.
Lewis & Murdoch (2005). Am J Pathol 167:627-635.
Liu et al. (1998) Proc Natl Acad Sci USA 95:10626-10631.
Li et al. (2007). Mol Cell 26: 63-74.
Li et al. (2009). Cancer Cell 15: 501-513.
Liao et al. (2007). Cancer Res 67: 563-572.
Maltepe et al. (1997). Nature 386: 403-407.
Maltepe et al. (1997). Nature 386:403-407.
Mateo et al. (2003) Biochem J 376: 537-544.
Matrone et al. (2004). J Neurochem 90:368-378.
Maxwell et al. (1997) Proc Natl Acad Sci USA 94: 8104-8109.
Maxwell et al. (1999). Nature 399: 271-275.
Maxwell et al. (1999). Nature 399:271-275.
Maxwell et al. (2001). Adv Exp Med Biol 502: 365-376.
Maxwell et al. (2001). Adv Exp Med Biol 502:365-376.
Melillo (2004). Cell Cycle 3:154-155.
Melillo et al. (1997). J Biol Chem 272:12236-12243.
Metzen et al. (2003) Mol Biol Cell 14: 3470-3481.
Metzen et al. (2003). Mol Biol Cell 14:3470-3481.
Min et al. (2002). Science 296: 1886-1889.
Minchinton & Tannock (2006) Nat Rev Cancer 6: 583-592.
Mitra et al. (2006) Mol Cancer Ther 5: 3268-3274.
Moeller (2004). Cancer Cell 5: 429-441.
Moeller et al. (2004). Cancer Cell 5:429-441.
Moeller et al. (2005). Cancer Cell 8: 99-110.
Moeller et al. (2005). Cancer Cell 8:99-110.
Muerkoster et al. (2006) Oncogene 25: 3973-3981.
Muller et al. (2005). Nat Med 11:312-319.
Nagasawa et al. (1995) Cancer Res 55: 1842-1846.
Naka et al. (1997) Nature 387:924-929
Nardinocchi et al. (2009) Mol Cancer 8: 1.
Needleman & Wunsch (1970) J Mol Biol 48:443-453.
Ohh et al. (2000). Nat Cell Biol 2: 423-427.
Ohh et al. (2000). Nat Cell Biol 2:423-427.
Park et al. (2008) Mol Pharmacol 74: 236-245.
Pause et al. (1999). Proc Natl Acad Sci USA 96: 9533-9538.
Pause et al. (1999). Proc Natl Acad Sci USA 96:9533-9538.
PCT International Publication Nos. WO 97/47763; WO 99/07409; WO 99/32619; WO 00/01846; WO 00/44895; WO 00/44914; WO 01/36646; and WO 01/29058.
Pearson & Lipman (1988) Proc Natl Acad Sci USA 85:2444-2448.
Peyssonnaux et al. (2005). J Clin Invest 115:1806-1815.
Primeau et al. (2005) Clin Cancer Res 11: 8782-8788.
Rankin & Giaccia (2008) Cell Death Differ 15: 678-685.
Rapisarda et al. (2002). Cancer Res 62:4316-4324.
Ravi et al. (2000). Genes Dev 14: 34-44.
Ravi et al. (2000). Genes Dev 14:34-44.
Rose & Botstein (1983) Meth Enzymol 101:167-180.
Rottenberg et al. (2007) Proc Natl Acad Sci USA 104: 12117-12122.
Samardzic (2001) Cytokine 13: 179-182.
Sambrook & Russell (2001) Molecular Cloning: A Laboratory Manual, 3rd ed. Cold Spring Harbor Laboratory Press, Cold Spring Harbor, N.Y., United States of America.
Sanchez-Puig et al. (2005). Mol Cell 17:11-21.
Sandau et al. (2001) Blood 97: 1009-1015.
Scharfmann et al. (1991) Proc Natl Acad Sci USA 88:4626-4630.
Selak et al. (2005). Cancer Cell 7:77-85.
Semenza (2002). Trends Mol Med 8:S62-67.
Semenza (2003) Nat Rev Cancer 3:721-32.
Semenza (2003). Nat Rev Cancer 3: 721-732.
Semenza et al. (1997). Kidney Int 51: 553-555.
Semenza et al. (2000). Adv Exp Med Biol 475:123-130.
Semenza G L (2000) Cancer Metastasis Rev 19: 59-65.
Shaked et al. (2008). Cancer Cell 14: 263-273.
Silhavy et al. (1984) Experiments with Gene Fusions. Cold Spring Harbor Laboratory, Cold Spring Harbor, N.Y., United States of America.
Smith & Waterman (1981) Adv Appl Math 2:482-489.
Sogawa et al. (1998). Proc Natl Acad Sci USA 95:7368-7373.
Song et al. (2006) Cancer Chemother Pharmacol 58: 776-784.
Sperl et al. (2009). Bioorg Med Chem Lett 19:3305-3309
Sumbayev et al. (2003). FEBS Lett 535:106-112.
Sutphin et al. (2004). Cell Cycle 3:160-163.
Sweeney et al. (2001) Cancer Res 61: 3369-3372.
Talks et al. (2000) Am J Pathol 157: 411-421.
Thomas et al. (2004) Cancer Res 64: 8357-8364.
Thomsen et al. (1997). Cancer Res 57:3300-3304.
Tijssen (1993) Laboratory Techniques in Biochemistry and Molecular Biology-Hybridization with Nucleic Acid Probes. Elsevier, New York, United States of America.
Tran et al. (2002) Proc Natl Acad Sci USA 99: 4349-4354.
U.S. Pat. Nos. 5,114,951; 5,410,016; 5,411,554; 5,468,253; 5,573,934; 5,599,852; 5,631,015; 5,653,992; 5,688,900; 5,713,920; 5,728,752; 5,824,333; 5,858,746; 5,858,784; 6,013,638; 6,022,737; 6,136,295; 7,009,034; 7,011,842; and U.S. Pat. No. 7,012,126.
Udono et al. (1994). Proc Natl Acad Sci USA 91:3077-3081.
Unruh et al. (2003) Oncogene 22: 3213-3220.
Vanhoefer et al. (1997) Ann Oncol 8: 1221-1228.
Varney et al. (2002). In Vivo 16:471-477.
Wachsberger et al. (2003). Clin Cancer Res 9:1957-1971.
Wang & Semenza (1993) J Biol Chem 268: 21513-21518.
Wang & Semenza (1993a). J Biol Chem 268:21513-21518.
Wang & Semenza (1993b). Proc Natl Acad Sci USA 90:4304-4308.
Wang & Semenza (1995). J Biol Chem 270:1230-1237.
Wang et al. (1998) Int J Mol Med 1: 915-923.
Wang et al. (2004) Blood 104: 2893-2902.
Wang G L, Semenza G L (1995). J Biol Chem 270: 1230-1237.
Wartenberg et al. (1998) Int J Cancer 75: 855-863.
Weigand et al. (2005) Angiogenesis 8: 197-204.
Williams et al. (1993) J Clin Invest 92:503-508.
Yamaguchi et al. (2005) Oncology 68: 471-478.
Yasinska & Sumbayev (2003). FEBS Lett 549:105-109.
Yeo et al. (2003). J Natl Cancer Inst 95:516-525.
Yu et al. (1998). Am J Physiol 275:L818-826.
Yu et al. (1999) Cancer Res 59:4200-4203.
Zhang et al. (2004a). Blood 103:617-626.
Zhang et al. (2004b). Cancer Res 64:8139-8142.
Zhong et al. (2000). Cancer Res 60:1541-1545.
Zundel et al. (2000). Genes Dev 14:391-396.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for implementing a treatment strategy for a cancer in a subject in need of treatment, the method comprising:
a) administering to the subject a chemotherapeutic agent;
b) determining a level of HIF-1 activity in a cancer tissue in the subject; and
c) administering an inhibitor of HIF-1 activity to the subject if an elevation of HIF-1 activity is observed after administering the chemotherapeutic agent.

2. The method of claim 1, wherein the cancer tissue expresses inducible nitric oxide synthase (iNOS).

3. The method of claim 1, wherein the cancer tissue in which HIF-1 activity is evaluated is normoxic cancer tissue.

4. The method of claim 1, wherein the level of HIF-1 activity in the cancer tissue is determined by detecting a plasma level of plasminogen activator inhibitor-1 (PAI-1).

5. The method of claim 1, wherein the level of HIF-1 activity in the cancer tissue is determined by establishing a ratio between PAI-1 and osteopontin (OPN).

6. The method of claim 1, wherein the inhibitor of HIF-1 activity is selected from the group consisting of a nitric oxide synthase inhibitor, a nitric oxide scavenger, a STAT-1 inhibitor, and combinations thereof.

7. The method of claim 6, wherein the nitric oxide synthase inhibitor comprises an iNOS inhibitor.

8. The method of claim 1, wherein the chemotherapeutic agent acts by oxidative stress.

9. The method of claim 8, wherein the chemotherapeutic agent comprises doxorubicin.

10. The method of claim 1, wherein step b) is performed within 1 to 10 days after performing step a).

11. The method of claim 10, wherein step c) is performed within 1 to 10 days after performing step b).

12. The method of claim 1, wherein the subject is a mammal.

13. The method of claim 12, wherein the mammal is a human.

14. A method of treating a cancer in a subject, the method comprising:
a) administering a chemotherapeutic agent to the subject;
b) determining whether administering the chemotherapeutic agent to the subject elevates HIF-1 activity in cancer tissue of the subject; and
c) administering a HIF-1 inhibitor to the subject depending on whether elevated HIF-1 activity is observed in the cancer tissue.

15. The method of claim 14, wherein the cancer tissue expresses inducible nitric oxide synthase (iNOS).

16. The method of claim 14, wherein the cancer tissue in which HIF-1 activity is evaluated is normoxic cancer tissue.

17. The method of claim 14, wherein the level of HIF-1 activity in the cancer tissue is determined by detecting a plasma level of plasminogen activator inhibitor-1 (PAI-1).

18. The method of claim 14, wherein the level of HIF-1 activity in the cancer tissue is determined by establishing a ratio between PAI-1 and osteopontin (OPN).

19. The method of claim 14, wherein the HIF-1 inhibitor is selected from the group consisting of a nitric oxide synthase inhibitor, a nitric oxide scavenger, a STAT-1 inhibitor, and combinations thereof.

20. The method of claim 19, wherein the nitric oxide synthase inhibitor comprises an iNOS inhibitor.

21. The method of claim 14, wherein the chemotherapeutic agent acts by oxidative stress.

22. The method of claim 21, wherein the chemotherapeutic agent comprises doxorubicin.

23. The method of claim 14, wherein step b) is performed within 1 to 10 days after performing step a).

24. The method of claim 23, wherein step c) is performed within 1 to 10 days after performing step b).

25. The method of claim 14, wherein the subject is a mammal.

26. The method of claim 25, wherein the mammal is a human.


SEQUENCE LISTING (25 sequences)

SEQ ID NO: 1 - protein, 824 aa, Spalax judaei
MEGAGGENEKKKMSSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGPVKEGKEQNTQRSFFLRMKCTLTSRGRTMNIKSATWKVLHCTGRIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSCLFDKLKKEPDALTMLAPAAGDTIISLDFGSDDTETEDQQLEDVPLYNDVMFPSSDDKLTSINLAMSPLPAPETPKPLRSNADPALNQEVALKLEPNAESLELSFTMPQIQDQPASPSDGSTRQSSPEPNSPSEYCFDVDSDMVNVFKLELVEKLFAEDTEAKNPFSTQDTDLDLEMLAPYIPMDDDFQLRLFDQLSPLESSSPSPPGVNTATAFQQTQLQEPTISTTTTTTTTNELKTVTKDNIEDIKILIASPSSTHTPKETTTATTSSPYSGTQSRTASPNRAGQGVIEQTEKSHPRSPNLLSVSLSQRNTVPEEELNPKIIALQNAQRKRKMEHDGSLFQAAGIGTLLQQPDDRAPATSLSWKRVKGCKSSGQNGMEQKTIILIPSDLACRLLGQSMDGSGLPQLTSYDCEVNAPIQGSRNLLQGEELLRALDQVN

SEQ ID NO: 2 - protein, 819 aa, Eospalax baileyi
MEGAAGGEEKKNRMSSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGPIKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSCLFDKLKKEPDALTLLAPAAGDTIISLDFGSDDTETEDQQLEDVPLYNDVMFPSSDDKLTSINLAMSPLPASETPKPLRSNADPALNQEVALKLEPNAESLELSFTMPQIQDQPASPSDGSTRQSSPEPNSPSEYCFDVDSDMVNVFKLELVEKLFAEDTEAKNPFSTQDTDLDLEMLAPYIPMDDDFQLRSFDQLSPLESSSPNPPSVSTAFQQTQLQEPTITTTTTEELKTVTKDSTEDIKILITSPSSTHTPKETTTATTSSPYSGTQSRTASPNRAGQGVIEQTEKSHPRSPNVLSVTLSQRNTVPEEELNPKIIALQNAQRKRKMEHDGSLFQAAGIGTLLQQPDDRAPATSLSWKRVKGCKSSGQNGMEQKTIILIPSDLACRLLGQSMDGSGLPQLTSYDCEVNAPIQGSRNLLQGEELLRALDQVN

SEQ ID NO: 3 - protein, 836 aa, Mus musculus
MEGAGGENEKKKMSSERRKEKSRDAARSRRSKESEVFYELAHQLPLPHNVSSHLDKASVMRLTISYLRVRKLLDAGGLDSEDEMKAQMDCFYLKALDGFVMVLTDDGDMVYISDNVNKYMGLTQFELTGHSVFDFTHPCDHEEMREMLTHRNGPVRKGKELNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPSSETPKPLRSSADPALNQEVALKLESSPESLGLSFTMPQIQDQPASPSDGSTRQSSPERLLQENVNTPNFSQPNSPSEYCFDVDSDMVNVFKLELVEKLFAEDTEAKNPFSTQDTDLDLEMLAPYIPMDDDFQLRSFDQLSPLESNSPSPPSMSTVTGFQQTQLQKPTITATATTTATTDESKTETKDNKEDIKILIASPSSTQVPQETTTAKASAYSGTHSRTASPDRAGKRVIEQTDKAHPRSLKLSATLNQRNTVPEEELNPKTIASQNAQRKRKMEHDGSLFQAAGIGTLLQQPGDCAPTMSLSWKRVKGFISSEQNGTEQKTIILIPSDLACRLLGQSMDESGLPQLTSYDCEVNAPIQGSRNLLQGEELLRALDQVN

SEQ ID NO: 4 - protein, 823 aa, Rattus norvegicus
MEGAGGENEKKNRMSSERRKEKSRDAARSRRSKESEVFYELAHQLPLPHNVSSHLDKASVMRLTISYLRVRKLLGAGDLDIEDEMKAQMNCFYLKALDGFVMVLTDDGDMIYISDNVNKYMGLTQFELTGHSVFDFTHPCDHEEMREMLTHRNGPVRKGKEQNTQRSFFLRMKCTLTSRGRTMNIKSATWKVLHCTGHIHVYDTSSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPASETPKPLRSSADPALNQEVALKLESSPESLGLSFTMPQIQDQPASPSDGSTRQSSPEPNSPSEYCFDVDSDMVNVFKLELVEKLFAEDTEAKNPFSAQDTDLDLEMLAPYIPMDDDFQLRSFDQLSPLESNSPSPPSVSTVTGFQQTQLQKPTITVTATATATTDESKAVTKDNIEDIKILIASPPSTQVPQEMTTAKASAYSGTHSRTASPDRAGKRVIEKTDKAHPRSLNLSVTLNQRNTVPEEELNPKTIALQNAQRKRKMEHDGSLFQAAGIGTLLQQPGDRAPTMSLSWKRVKGYISSEQDGMEQKTIFLIPSDLACRLLGQSMDESGLPQLTSYDCEVNAPIQGSRNLLQGEELLRALDQVN

SEQ ID NO: 5 - protein, 828 aa, Microtus oeconomus
MEGAGGENEKKKMSSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGPVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSCLFDKLKKEPDALTLLAPAAGDTIISLDFGSDDTETEDQQLEDVPLYNDVMFPSSNEKLTNINLALSPLPASETPKPLRSSADPALNQEVALKLEPSPESLELSFTMPQIQDQPASPSDGSTRQSSPEPNSPSEYCFDVDSDMVNVFKLELVEKLFAEDTEAKNPFSTQDTDLDLEMLAPYIPMDDDFQLRSFDQLSPLESSSSSPPSVSTVTGFQQTPLQKPTITAAAATTATTTATTTDELKTVTKDNIEDIKILIASPSSVHVPQETTAATASAYSAPHSRTASPDRAGKRVREQAEKAHPRSPNMSVTLSQRNTVTEEDLNPKIIALQNAQRKRKMEHDGSLFQASGIGTLLQQPGDRAPTTSLSWKRVKGCKSNEQNGMEQKTIILIPSDLACRLLGQSMDESGLPQLTSYDCEVNAPVQGSRNLLQGEDLLRALDQVN

SEQ ID NO: 6 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 7 - protein, 827 aa, Pongo pygmaeus
MEGAGGANDKKNRISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGPVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQPPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPEDHAATTSLSWKRVKGCKSSEQNGMEQKTIILIPSDLACRLLGQSMDESGLPQLTSYDCEVNAPIQGSRNLLQGEELLRALDQVN

SEQ ID NO: 8 - protein, 826 aa, Macaca fascicularis
MEGAGGANDKKKISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCGHEEMREMLTHRNGPVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHGMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSSNEKLQNINLAMSPLPTSETPKPLRSSADPALNQEVALKLEPNPESLELSFTMPQIQDQPPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSSTHIHKETTSATSSPYRDTQSRTASPNRAGKGVIEQTEKSHPRSPNVLSVTLSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 9 - protein, 821 aa, Spermophilus tridecemlineatus
  misc_feature (49)..(49): Xaa can be any naturally occurring amino acid
MEGAGGTNDKKKISSERRKEKSRDAARSRRSKESEVFYELAHQLPLPHXVSSHLDKASVMRLTISYLLVRKLLDAGDLDIEDEMKAQMNCFYLKALDGFVMVLTDDGDMIYISDNVNKYMGLTQFELTGHSVFDFTHPCDHEEMREMLTHRNGPVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHGLIFSLQQTECVLKPVESSDMKMTQLFTKVESEDTSSLFDKLKKEPDALTLLAPAAGDTIISLDFGSNDTETEDQQLEEVPLYNDVMFPSSSEKLQNINLAMSPLPASETPKPLRSSADPALNQEVALKLEPNPESLELSFTMPQIQDQPASPSDGSTRQSSPEPNSPSEYCFDVDSDMVNVFKLELVEKLFAEDTEAKNPFSTQDTDLDLEMLAPYIPMDDDFQLRSFDQLSPLESSSSSPQSVSTISVFQQTQIQEPTINTTTATTDELKTVTKDDMEDIKILIASWSPTHAPKETTSATTSSYNDTQSRTASPNRAGKEVIEQTEKSHPRSPNVVSVTLSQRNTVPEEELNPKILALQNAQKKAKMEQDGSLFQAVGIGTLLQQPDDRATTTSLSWKRVKGCKSSEQNGMEQKTIILIPSDLACRLLGQSMDESGLPQLTSYDCEVNAPIQGSRNLLQGEELLRALDQVN

SEQ ID NO: 10 - protein, 823 aa, Bos taurus
MEGAGGANDKKKISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWIETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSSNEKLQNINLAMSPLPASETPKPLRSSADPALNQEVALKLEPNPESLELSFTMPQIQDQPASPSDGSTRQSSPEPNSPSEYCFDVDSDMVNEFKLELVEKLFAEDTEAKNPFSTQDTDLDLEMLAPYIPMDDDFQLRSFDQLSPLENSSTSPQSASTNTVFQPTQMQKPPIATVTTTATSDELKTVTKDGMEDIKILIAFPSPPHVPKEPPCATTSPYSDTGSRTASPNRAGKGVIEQTEKSHPRSPNVLSVALSQRTTAPEEELNPKILALQNAQRKRKIEHDGSLFQAVGIGTLLQQPDDRATTTSLSWKRVKGCKSSEQNGMEQKTIILIPSDLACRLLGQSMDESGLPQLTSYDCEVNAPIQGSRNLLQGEELLRALDQVN

SEQ ID NO: 11 - protein, 823 aa, Pantholops hodgsonii
MEGAGGANDKKKISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWIETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDAETDDQQLEEVPLYNDVMLPSSNEKLQNINLAMSPLPASETPKPLRSSADPALNQEVALKLEPNPESLELSFTMPQIQDQPASPSDGSTRQSSPEPNSPSEYCFDVDSDMVNEFKLELVEKLFAEDTEAKNPFSTQDTDLDLEMLAPYIPMDDDFQLRSFDQLSPLENSSTSPQSASTDTVFQPTQMQEPPIATATTTATNDELKTVTKDGMEDIKILIAFPSPPHVPKEPPCATTSPYSDTGSRTASPNRAGKGVIEQTEKSHPRSPNVLSVALSQRTTAPEEELNPKILALQNAQRKRKIEHDGSLFQAVGIGTLLQQPDDRATTTSLSWKRVKGCKSSEQNGMEQKTIILIPSDLACRLLGQSMDESGLPQLTSYDCEVNAPIQGSRNLLQGEELLRALDQVN

SEQ ID NO: 12 - protein, 823 aa, Canis familiaris
MEGAGGANDKKKISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSSNEKLQNINLAMSPLPASETPKPLRSSADPALNQEVALKLEPNPESLELSFTMPQIQDQPASPSDGSTRQSSPEPNSPSEYCFDVDSDMVNEFKLELVEKLFAEDTEAKNPFSTQDTDLDLEMLAPYIPMDDDFQLRSFDQLSPLESNSTSPQSASTITVFQPTPMQEPPLTTTSTTATTDELKTVTKDGIEDIKILIAAPSPTHVPKVTTSATTSPYSDTGSRTASPNRAGKGVIEQTEKSHPRSPNVLSVTLSQRTTIPEEELNPKILALQNAQRKRKIEHDGSLFQAVGIGTLLQQPDDRATTTSLSWKRVKGCKSSEQNGMEQKTIILIPSDLACRLLGQSMDESGLPQLTSYDCEVNAPIQGSRNLLQGEELLRALDQVN

SEQ ID NO: 13 - protein, 819 aa, Oryctolagus cuniculus
MEGAGGANDKKKISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGPVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESADTSSLFDKLKKEPDALTLLAPAAGDTIISLDFGSNDTETDDQQLEEVPLYNDVMFPSSNEKLQDINLAMSPLPASESPKPLRSSADPALNQEVALKLEPNPESLELSFTMPQIQDQPASPSDGSTRQSSPEPTSPSEYCFDVDSDMVNVFKLELVEKLFAEDTEAKNPFSTQDTDLDLEMLAPYIPMDDDFQLRSFDQLSPLESSSAGAVTVFPQTQGQEPAANTAPCAPPPLTIKTSAKDSVEDMKVLIASPSPNHIPKETASATTSPPYRDTPSRTASPNTAGKGVIEQTEKSHPRSPNVLAVTLSQRTTAPEEELNPKILALQNAQRKRKMESDGSLFQAVGIGTLLQQPDDRTTTASLSWKRVKGCKSSEQNGMEQKTIILIPSDLACRLLGQSMDESGLPQLTSYDCEVNAPIQGSRNLLQGEELLRALDQVN

SEQ ID NO: 14 - protein, 811 aa, Gallus gallus
MDSPGGVTDKKRISSERRKEKSRDAARCRRSKESEVFYELAHQLPLPHTVSAHLDKASIMRLTISYLRMRKLLDAGELETEANMEKELNCFYLKALDGFVMVLSEDGDMIYMSENVNKCMGLTQFDLTGHSVFDFTHPCDHEELREMLTHRNGPVKKGKEQNTERSFFLRMKCTLTSRGRTVNIKSATWKVLHCTGHIRVYDTCNNQTHCGYKKPPMTCLVLICEPIPHPSNIEVPLDSKTFLSRHSLDMKFSYCDERITELMGYEPEELLGRSIYEYYHALDSDHLTKTHHDMFTKGQVTTGQYRMLAKQGGYVWVETQATVIYNTKNSQPQCIVCVNYVLSGIVQKDLIFSLGQTECMLKPVESPEMKMTKIFSKDDWDDTNSLFEKLKQEPDALTVLAPAAGDTIISLDFSSNESDEQQCDEVPLYNDVMLPSSSEKLQNINIAMSPLPASETTKPLRSNADPALNREVVSKLEPNTETLELSFTMPQVQEQPTSPSDASTSQSSPEPSSPNDYCFDVDNDMANEFKLELVEKLFAIDTEAKNPFSTQETDLDLEMLAPYIPMDDDFQLRSFDQLSPLESSSSGSQNAATITILQQTQTPSTAADEIKPVAERVDDVKALIVPSSPVHVINDTSSAPASPYSGNRSRTASPIRAGKGTLEQTEKSCPGAPSLITVTLNKRSTAMDEELNPKMLALHNAQRKRKMEHDGSLFQAVGIGSLFQQTGDRGGNASLAWKRVKACKTNGHNGVEQKTIILLSTDIASKLLGQSMDESGLPQLTSYDCEVNAPIQGNRNLLQGEELLRALDQVN

SEQ ID NO: 15 - protein, 533 aa, Danio rerio
MDTGVVTEKKRVSSERRKGKSRDAARSRRGKESEVFYELAHQLPLPHNVTSHLDKASIMRLTISYLRMRKLLNSDEKEEKEENELESQLNGFYLKALEGFLMVLSEDGDMVYLSENVSKSMGLTQFDLTGHSIFEFSHPCDHEELREMLVHRTGSKKTKEQNTERSFFLRMKCTLTSRGRTVNIKSATWKVLHCAGHVRVHEGSEASEDSGFKEPPVTYLVLICEPIPHPSNIEVPLDSKTFLSRHTLDMKFSYCDERITELMGYEPDDLLNRSVYEYYHALDSDHLTKTHHNLFAKGQATTGQYRMLAKKGGFVWVETQATVIYNPKNSQPQCIVCVNYVLSGIVEGDVVLSLQQTVTEPKAVEKESEETEEKTSELDILKLFKPESLNCSLESSTLYNKLKEEPEALTVLAPAAGDAIISLDFNNSDSDIQLLKEVPLYNDVMLPSSSEKLPLSLSPLTPSDSLSSHATTAKSTLPCRRRHPGPLHPYTCCRRCAVHLSRSSVAVGMPHLFDPAPHRAAVSSTTEKCLQRC

SEQ ID NO: 16 - protein, 805 aa, Xenopus laevis
MEGSVVVSEKKRISSERRKEKSRDAARCRRSNESEVFYELSHELPLPHNVSSHLDKASIMRLDHQLPAVEKVADAGDLDGETELDKQLNCFYLKALEGFVLVLTEEGDMIYLSENVNKCMGLTQFELTGHSVFDFTHPCDHEELREMLTFRNGPAKKRKRTNHREKFLPSYEMYINQSWKNREYKVSHMEGPSLYRTHACIYDNANNQNHCGYKKPPMTCMVVICEPIPHPSNIEFPLDSKTFLSRHSLDMKFSYCDERVTELVGYEPDELLGRSVYEYYHALDSDHLTKPNYNMFTKGQVTTGQYRMLAKKGGYVWVETQATVIYNSKNSQPQCIVCVNYVLSEVVEKDLILSLGQTASVLIPVESQEIKMPEIFTELNEENNSECLFDKLKQEPESLTVLAPDAGDEIIPLDFSSGDSDKPYEDVPLYNDVMLHSTSNKLESTPITPLPAPEMPKPLRSNVDPALNREVVIKMESNPRTTCASIHHSTAIQARQPFRYQFQSEPSTEPNTPEYCFDVDSEMASEFKLDLVEKLFAIDTEAKAPFYYPGNDLDLEMLAPYIPMDDDFQLRTFDQLSSLECDSSIPQTLGSMTTLFHQSLSPSTSDFKPEDAMSDLKTIIQSPVHMMKESTSAPVSPYNGNRSRTSSPVRPAKAVVDKTEKSRPGTPNLPVPLNKRCTILDEELNPKMICFTQCTAEKRKMESDGPLFQAIGIGTLFQTNVDPGPNSSLQWKRVKGSDSERLSSAEQRTILLLSTDMASQLLGQSFDGTVLPQLTGYDCEVNAPVHGTRNLLQGEELLRALDQAN

SEQ ID NO: 17 - DNA, 21 bp, Artificial Sequence
  siRNA directed against mouse VHL factor, including 3′ AT
aacatcacattgccagtgtat

SEQ ID NO: 18 - protein, 15 aa, Homo sapiens
PNSPSEYCFYVDSDM

SEQ ID NO: 19 - protein, 15 aa, Mus musculus
PNSPSEYCFDVDSDM

SEQ ID NO: 20 - protein, 15 aa, Carassius carassius
PNSPMEYCFQVDSDI

SEQ ID NO: 21 - protein, 15 aa, Xenopus laevis
EPNTPEYCFDVDSEM

SEQ ID NO: 22 - DNA, 24 bp, Artificial Sequence
  primer
ctgtgagacctttgatgtccgaag

SEQ ID NO: 23 - DNA, 24 bp, Artificial Sequence
  primer
ctggatgagcctatattgctgtgg

SEQ ID NO: 24 - DNA, 22 bp, Artificial Sequence
  primer
gattactgctctggctcctagc

SEQ ID NO: 25 - DNA, 22 bp, Artificial Sequence
  primer
gactcatcgtactcctgcttgc